SEQUENCE LISTING (4 sequences)

SEQ ID NO: 1 - protein, 93 aa, Homo sapiens
MLTELEKALNSIIDVYHKYSLIKGNFHAVYRDDLKKLLETECPQYIRKKGADVWFKELDINTDGAVNFQEFLILVIKMGVAAHKKSHEESHKE

SEQ ID NO: 2 - DNA, 352 bp, Homo sapiens
atgtctcttgtcagctgtctttcagaagacctggttctgtttttcaggtggggcaagtccgtgggcatcatgttgaccgagctggagaaagccttgaactctatcatcgacgtctaccacaagtactccctgataaaggggaatttccatgccgtctacagggatgacctgaagaaattgctagagaccgagtgtcctcagtatatcaggaaaaagggtgcagacgtctggttcaaagagttggatatcaacactgatggtgcagttaacttccaggagttcctcattctggtgataaagatgggcgtggcagcccacaaaaaaagccatgaagaaagccacaaagagtagc

SEQ ID NO: 3 - protein, 114 aa, Homo sapiens
MTCKMSQLERNIETIINTFHQYSVKLGHPDTLNQGEFKELVRKDLQNFLKKENKNEKVIEHIMEDLDTNADKQLSFEEFIMLMARLTWASHEKMHEGDEGPGHHHKPGLGEGTP

SEQ ID NO: 4 - DNA, 345 bp, Homo sapiens
atgacttgcaaaatgtcgcagctggaacgcaacatagagaccatcatcaacaccttccaccaatactctgtgaagctggggcacccagacaccctgaaccagggggaattcaaagagctggtgcgaaaagatctgcaaaattttctcaagaaggagaataagaatgaaaaggtcatagaacacatcatggaggacctggacacaaatgcagacaagcagctgagcttcgaggagttcatcatgctgatggcgaggctaacctgggcctcccacgagaagatgcacgagggtgacgagggccctggccaccaccataagccaggcctcggggagggcaccccctaa

(19) United States
(12) Patent Application Publication
(10) Pub. No.: US 2023/0151434 A1
(43) Pub. Date: May 18, 2023

(54) REAWAKENING OF DORMANT TUMOR CELLS BY MODIFIED LIPIDS DERIVED FROM STRESS ACTIVATED NEUTROPHILS

(71) Applicant: The Wistar Institute of Anatomy and Biology, Philadelphia, PA (US)

(72) Inventor: Dmitry I. Gabrilovich, Villanova, PA (US)

(21) Appl. No.: 17/995,539

(22) PCT Filed: Apr. 7, 2021
(86) PCT No.: PCT/US2021/026190

§ 371 Date: Oct. 5, 2022

Related U.S. Application Data
(60) Provisional application No. 63/006,312, filed on Apr. 7, 2020.

(51) Int. Cl. C12Q 1/6886; A61P 35/00

(52) U.S. Cl. CPC C12Q 1/6886; A61P 35/00

(57) ABSTRACT

Provided herein are methods of method of inhibiting the recurrence of cancer in subject associated with stress-induced β-adrenergic pathway signaling. In certain embodiments, the methods include treating a subject with an inhibitor of S100A8/A9.

### STATEMENT REGARDING FEDERALLY SPONSORED RESEARCH OR DEVELOPMENT

This invention was made with government support under grant numbers CA165065 and P50 CA168536 awarded by the National Institutes of Health and grant number LC180388 awarded by the Department of Defense. The government has certain rights in the invention.

### BACKGROUND

Tumor recurrence years after complete surgical resection or complete clinical response to chemo- or radiation therapy is one of the major causes of cancer-related deaths. Cancer cell dissemination is likely to happen early during primary cancer evolution prior to initial therapy (Perego et al., 2018; Rhim et al., 2012). Disseminated tumor cells can lie dormant for extended time before initiating metastatic outgrowth. Cancer cell dormancy encompasses two major conditions: quiescence and senescence. Quiescence is the reversible state of a cell arrest in which cells retain the ability to re-enter cell cycle. Senescence is a stress response that can be induced by a wide range of intrinsic and extrinsic factors including p53/p21^{cip}1 pathway, radiation, or chemotherapy (Ewald et al., 2010). Cellular senescence is a dynamic process that includes several phases (van Deursen, 2014). Senescent cancer cells have enlarged size, accumulation of DNA damage foci and increased activity of senescence-associated β-galactosidase (SA-β-gal) (Herranz and Gil, 2018). DNA-damage response induces cell cycle arrest either in G1 or more often in G2/M stage of cell cycle (Denoyelle et al., 2006). Senescent cells can persist for a long time in organs (Yang et al., 2017), can activate stemness programs, and acquire higher tumorigenicity than original non-senescent cells (Milanovic et al., 2018). In contrast to replicative senescence of normal cells, which is irreversible, tumor cell senescence induced by oncogenes, chemotherapy or radiation therapy can be reversed (Chakradeo et al., 2016). However, the mechanism by which tumor cell senescence is reversed has not been previously elucidated.

What is needed are therapies that prevent dormant cancer cells from becoming metastatic.

### SUMMARY OF THE INVENTION

In one aspect, provided herein is a method useful for inhibiting reactivation of dormant tumor cells in a subject previously diagnosed with cancer. In one embodiment, the method includes inhibiting or reducing S100A8/A9 in a subject. In certain embodiments, the method comprises administering S100A8/A9 inhibitor to a subject in the need thereof.

In another aspect, the method comprises inhibiting or reducing FGFR in a subject. In some embodiments, the FGFR is selected from: FGFR1, FGFR2, and/or FGF7.

In another aspect, the method comprises inhibiting or reducing myeloperoxidase (MPO) in the subject.

In another aspect, a method is provided wherein levels of S100A8 or S100A9 in samples obtained from subject are compared to a control, wherein an increase in levels of S100A8 or S100A9 indicates a greater risk of presence of reactivated dormant tumor cells in a subject. The method comprises treating the subject with an inhibitor of S100A8 or S100A9, FGFR and/or MPO, when a greater risk of reactivation is detected. In one aspect, the levels of S100A8 or S100A9 is 2500 ng/mL or higher and are indicative of an increased risk of reactivation of dormant tumor cells in the subject. In another aspect, the method comprises co-administering additional composition for treatment of the subject. The co-administered agent may be a chemotherapeutic agent.

In another aspect, a method of inhibiting the recurrence of cancer in subject associated with stress-induced β-adrenergic pathway signaling is provided. The method includes inhibiting or reducing S100A8/A9 in the subject. In another aspect, the method involves inhibiting the recurrence of cancer in a subject, wherein the method involves inhibiting stress-induced β-adrenergic pathway signaling.

In another aspect, a method is provided that includes identifying the presence of PMN-MDSC in a subject previously treated for cancer. Furthermore, the method includes treating the subject with an inhibitor of S100A8 or S100A9, FGFR or MPO when the presence of PMN-MDSC is detected.

Other aspects and advantages of the invention will be readily apparent from the following detailed description of the invention.

### DESCRIPTION OF THE FIGURES

FIG. 1A-FIG. 1H show polymorphonuclear myeloid-derived suppressor cells but not neutrophils reactivate dormant tumor cells. (FIG. 1A) Representative image of KPr tumor cells in culture before (left) and after sorting (right). Center panel represents the gating strategy to sort arrested (A) and proliferating (P) cells. Scale bar, 50 μm. (FIG. 1B) Example of proliferation measured by luciferase activity in KPr and KPr^{p53}A cells after 5 days of culture. Means±SEM are shown, n=3. (FIG. 1C) Fold increase in number of KPr^{p53}A cells cultured in the presence of Ly6G⁺ PMN-MDSC (isolated from LLC tumor-bearing mice) or Ly6G+ PMN (from nai\ddot{v}e mice) over KPr^{p53}A cells cultured alone. Data represented as means±SEM. Six independent experiments with 16 replicates each were performed and one experiment is presented. (FIG. 1D) Fold increase in number of KPr^{p53}A cells cultured with indicated cells at a 1:10 ratio over KPr^{p53}A cells cultured alone. Data represented as means±SEM of three independent experiments with 16 replicates each are shown. (FIG. 1E) Fold increase in the number of KPr^{p53}A cells cultured at a 1:10 ratio with PMN-MDSC isolated from spleens of WT or S100a9KO mice over KPr^{p53}A cells cultured alone. Means±SEM of four independent experiments with 16 replicates each are shown. For FIG. 1C-FIG. 1E, P values were calculated using one-way ANOVA with correction for multiple comparisons. (FIG. 1F) Representative images of β-galactosidase staining in reactivated KPr^{p53}A^{React} tumor cells. AT-3 cells treated with doxorubicin (20 nM) were used as a positive control. (FIG. 1G) Flow cytometry analysis of BrdU retention in KPr^{p53}A cells (red) and KPr^{p53React} cells (blue). (FIG. 1H) Top: Schema of the experiment. Bottom: Representative images of NOD/SCID mice intravenously injected with KPr^{p53}A cells and then with PMN or PMN-MDSC as indicated. Right: The number and proportion of mice in each group with detectable tumors. P values were calculated by Fisher's exact test.

FIG. 2A-FIG. 2K show neutrophil-mediated reactivation of dormant cells is regulated by stress-induced S100A8/A9. (FIG. 2A) Fold increase in the number of KPr^{p53}A cells cultured in the presence of PMN and S100A8/A9 or indicated cytokines relative to KPr^{p53}A cells cultured with PMN. Three independent experiments with 16 replicates were performed. Mean±SEM of one experiment is shown. (FIG. 2B) Fold increase in the number of KPr^{p53}A cells cultured in the presence of PMN and LPS relative to KPr^{p53}A cells cultured with PMN or alone. Means±SEM of three independent experiments with 16 replicates each are shown. (FIG. 2C) Fold increase in the number of KPr^{p53}A cells cultured in the presence of S100A8/A9 alone, PMN and S100A8/A9, or PMN-MDSC relative to KPr^{p53}A cultured alone. Means±SEM of 10 independent experiments with 16 replicates each are shown. (FIG. 2D) S100A8/A9 protein produced by PMN after treatment with indicated hormones or LPS was measured by ELISA. PMN-MDSC were isolated from spleens of Lewis lung carcinoma tumor-bearing mice. Means±SEM and results of each independent experiment with three replicates are shown. (FIG. 2E) S100A8/A9 protein secreted in vitro from PMN and PMN treated with NE with or without ICI-118; 553 as measured by ELISA. Means±SEM and results of independent experiment with three replicates are shown. (FIG. 2F) Flow cytometry staining of ADRB2 receptor (dark gray) on PMN isolated from mouse spleen. Isotype control, light gray. Representative histogram of three experiments is shown. (FIG. 2G) Fold increase in the number of KPr^{p53}A cells cultured with PMN alone, NE alone, PMN and NE together, and PMN and NE plus ICI-118; 553 relative to KPr^{p53}A cells cultured alone (Ctrl). Ten independent experiments with 16 replicates for each experiment were performed. Means±SEM in a representative experiment are shown. (FIG. 2H) Number of KPr^{p53}A cells cultured in the presence of PMN from S100a9KO mice alone or together with NE relative to KPr^{p53}A cells cultured alone (ctrl). Means±SEM are reported for 16 replicates. (FIG. 2I) Percentage of live PMN after overnight incubation with NE, S100A8/A9, or LPS. Each dot represents the average of three experimental replicates of one single experiment. Means±SEM are shown for each group. (FIG. 2J) Fold increase in number of LL2^{Cis}A cells cultured with PMN alone or together with S100A8/A9 or NE relative to LL2^{Cis}A cultured alone (Ctrl). Means±SEM of three independent experiments with 16 replicates each are shown. (FIG. 2K) Fold increase of cell counts of indicated mouse AT-3DoxoA and human A549^{Cis}A and OVCARCisA tumor cells cultured with PMN alone or together with S100A8/A9 or NE relative to tumor cells cultured alone (Ctrl). Means±SEM of three independent experiments with 16 replicates each. In all panels, P values were calculated using ANOVA test with correction for multiple comparisons.

FIG. 3A-FIG. 3E show systemic stress stimulates S100A8/A9 production from PMN and induces tumor reactivation. (FIG. 3A) Representative images of NOD/SCID mouse lungs and livers after injection of KPr^{p53}A cells and PMN and subjected to stress (left) or stress in the presence of ICI-118,551 (right). Graph summarizes the number and proportion of mice with detectable tumors. P values were calculated by Fisher's exact test. (FIG. 3B) Example of images of lungs injected with LL2^{Cis}A cells. The number and proportion of C57BL/6 mice with detectable tumors in the lungs are reported after injection of LL2^{Cis}A cells and subjected to stress. P values were calculated using Fisher's exact test. (FIG. 3C) Schema of the experiment with tasquinimod treatment. (FIG. 3D) Representative images of C57BL/6 mouse lungs after injection of LL2^{Cis}A cells and subjected to stress with or without treatment with tasquinimod. The number and proportion of mice with detectable tumors. P values were calculated using Fisher's exact test. (FIG. 3E) The number and proportion of WT and PAD4 KO mice with tumor lesions in lungs after injection of LL2^{Cis}A cells and subjected to stress.

FIG. 4A-FIG. 4H show the effect of stress on S100A8/A9 release by PMN. (FIG. 4A) Relative expression (to #i-actin) of s100a9 gene expression in PMN from stressed or nonstressed mice. Results of individual mice and means±SEM are shown. (FIG. 4B) S100A9 protein as measured by flow cytometry in PMN from spleen of stressed and control mice. Left: A typical example of staining. Right: Results of individual mice tested and means±SEM are shown. (FIG. 4C) S100A8/A9 protein concentration in plasma of C57BL/6 control or stressed mice as measured by ELISA. Results of individual mice and means±SEM are shown. (FIG. 4D) Fold increase in number of KPr^{p53}A cells cultured in the presence of PMN from stressed mice and recombinant S100A8/A9 over KPr^{p53}A cells cultured alone (ctrl). For FIG. 4A-FIG. 4D, P values were calculated by a two-sided Student's t test. (FIG. 4E) LL2 tumors were established subcutaneously in WT or S100A9 KO C57BL/6 mice. Tumors were resected when they became palpable, and 7 days later, mice were treated with cisplatin (5 mg/kg single dose intravenously). One week after cisplatin treatment, mice were exposed to stress. The number and proportion of mice with detectable tumors are reported. P values were calculated using Fisher's exact test. (FIG. 4F) MPO enzymatic activity in PMN stimulated with S100A8/A9 (5 μg/ml) or LPS (2 μg/ml). Results of individual mice (n=4 for LPS group; n=7 for two other groups) and means±SEM are shown. (FIG. 4G) Fold increase in number of KPr^{p53}A cells cultured in the presence of PMN from MPO KO mice alone or together with S100A8/A9 or NE relative to KPr^{p53}A cells cultured alone (ctrl). Means±SEM of three independent experiments with 16 replicates for each. (FIG. 4H) Fold increase in the number of KPr^{p53}A cells cultured in the presence of PMN and S100A8/A9 or PMN and NE over control (KPr^{p53}A cell cultured with PMN alone) in the presence or absence of MPO inhibitor (4-ABAH) at 2 μM concentration. Means±SEM of three independent experiments with 16 replicates each are shown. P values were calculated using ANOVA test with correction for multiple comparisons.

FIG. 5A-FIG. 5F show the effect of S100A8/A9 on PMN lipid content. (FIG. 5A) LC-ESI-MS/MS mass spectrometry of PMN. Results of individual mice (n=3) and means±SEM are shown. (FIG. 5B) PE-4-HNE Michael adduct of shown molecular species of PE in mouse WT and MPO KO PMN untreated or treated overnight with S100A8/A9. (FIG. 5C) Lyso-PE (LPE) species in mouse PMN treated with S100A8/A9. Means±SEM are shown; n=6 in untreated and S100A8/A9-treated PMN groups and n=3 in other groups. (FIG. 5D) PE-4-HNE Michael adduct molecular species of different PE in human PMN untreated or treated overnight with human recombinant S100A8/A9. (FIG. 5E) Oxidatively truncated PE in WT and MPO KO PMN untreated or treated overnight with S100A8/A9. (FIG. 5F) Lyso-PE containing saturated and monoenic acyl chain fatty acids in mouse PMN treated overnight with S100A8/A9 protein. In panels, results of individual experiments (n=3 to 6) and means±SEM are shown. In FIG. 5D, P values were calculated by a two-sided Student's t test. In all other panels, P values were calculated by ANOVA test with multiple comparison analysis. PE, phosphatidylethanolamine; PC, phosphatidylcholine; PS, phosphatidylserine; PI, phosphatidylinositol; PG, phosphatidylglycerol; BMP, bis(monoacylglycero)phosphate; PA, phosphatidic acid; CL, cardiolipin; SM, sphingomyelin.

FIG. 6A-FIG. 6H show that lipids from PMN treated with S100A8/A9 reactivate dormant tumor cells. (FIG. 6A) Fold increase in the number of KPr^{p53}A cells cultured with lipids (at the indicated concentrations) extracted from untreated PMN (untr) or PMN treated with S100A8/A9 (S100) relative to KPr^{p53}A cells cultured alone (ctrl). Means±SEM results of three independent experiments with 16 replicates for each are shown. (FIG. 6B) Increase in the number of KPr^{p53}A cells cultured with lipids extracted from MPO KO PMN treated with S100A8/A9 relative to KPr^{p53}A cells alone (ctrl). Means±SEM of three independent experiments with 16 replicates for each condition are shown. (FIG. 6C) Increase in the number of KPr^{p53}A cells cultured with lipids extracted from PMN isolated from control or stressed mice. Means±SEM of three independent experiments with 16 replicates for each condition are shown. (FIG. 6D) Increase in the number of LL2^{Cis}A cells cultured with lipids extracted from untreated PMN (untr) or PMN treated with S100A8/A9 (S100) at the indicated concentrations over LL2^{Cis}A cells cultured alone (ctrl). Top: PMN from wild-type mice. Bottom: PMN from MPO KO mice. Means±SEM of three independent experiments with 16 replicates for each are shown. (FIG. 6E) Increase in the number of LL2^{Cis}A cells cultured with lipids extracted from PMN from stressed C57BL/6 mice relative to the number of LL2^{Cis}A cells cultured alone (ctrl). Means±SEM of three independent experiments with 16 replicates for each. (FIG. 6F) Increase in number of human A549^{cis}A or OVCAR3^{cis}A cells treated with lipids extracted from human healthy donor PMN over tumor cells cultured alone (ctrl). Means±SEM of three independent experiments with 16 replicates for each condition are shown. (FIG. 6G) Increase in the number of KPr^{p53}A cells cultured with PE treated with MPO/H2O2/NaCl (MPO PE) over untreated KPr^{p53}A. Untreated (PE) or treated only with NaCl (NaCl PE) were used as control. PMN with S100A8/A9 were used as positive control. Means±SEM of three independent experiments with eight replicates for each condition are shown. (FIG. 6H) Increase in the number of KPr^{p53}A cells cultured with mixture of PE and PC treated with MPO/H2O2/NaCl over untreated KPr^{p53}A. Means±SEM of three independent experiments with eight replicates for each condition are shown. In all experiments, P values were calculated using ANOVA test with correction for multiple comparisons.

FIG. 7A-FIG. 7E show dormant cell reactivation and FGFR1 signaling in tumor cells. (FIG. 7A) Volcano plot with the log₂ and fold changes in gene expression between KPr and KPr^{p53}A^{React} on the x axis and the log₁₀ FDR on the y axis reveals genes with significant changes of expression in KPr^{p53}A^{React} over KPr cells as measured by RNA sequencing. (FIG. 7B) Heatmap of gene expression data showing the top pathways that were differentially expressed between KPr and KPr^{p53}A^{React} (left) and showing the different expression of genes associated to FGFR pathway (right). Fgf2 and Fgf7 are denoted by green dots. (FIG. 7C) qRT-PCR of relative Fgfr1 expression (top) and WB of FGFR1 protein (bottom) in KPr^{p53}A^{React} cells compared to KPr and KPr^{p53}P cells. Three experiments were performed. (FIG. 7D) qRT-PCR of Fgfr2 (left) and Fgf7 (right) expression. Means±SEM, n=4 in the KPr^{p53}A group, n=6 in the KPr^{p53}P group, and n=7 in the KPr^{p53}A^{react} group. (FIG. 7E) qRT-PCR expression of indicated genes in KPr^{p53}A cells untreated or treated overnight with lipids from PMN from stressed mice. Means±SEM, n=3. In all panels, P values were calculated using ANOVA test with correction for multiple comparisons.

FIG. 8A-FIG. 8E show the functional role of FGF signaling in PMN-mediated tumor cell reactivation from dormancy. (FIG. 8A) Number of tumor cells cultured in the presence of PMN and S100A8/A9 with or without BGJ398 FGFR inhibitor relative to tumor cells cultured alone. Means±SEM of three independent experiments with 16 replicates for each condition are shown. P values were calculated using ANOVA test with correction for multiple comparisons. (FIG. 8B) Representative images of C57BL/6 mouse lungs after injection of LL2^{Cis}A cells and subjected to stress with and without treatment with BGJ398. Graph summarizes the number and proportion of mice with detectable tumors. P values were calculated using Fisher's exact test. (FIG. 8C) Left: Proportion of patients with early recurrence of NSCLC among patients grouped on the basis of serum concentrations of S100A8/A9. P values were calculated using Boschloo's test. Right: Recurrence-free survival of patients grouped on the basis of serum concentration of S100A8/A9. P values were calculated using log-rank (Mantel-Cox) test. (FIG. 8D) Correlation between serum concentrations of S100A8/A9 and expression of S100A9 or the ratio of S100A9/FUT4 in frozen buffy coat cells (qRT-PCR). (FIG. 8E) Correlation between serum concentrations of S100A8/A9 and NE. In FIG. 8D and FIG. 8E, Spearman correlation coefficients and one-sided P values were calculated.

FIG. 9A-FIG. 9F show characterization of dormant tumor cells. (FIG. 9A) p53 expression in KPr cells in representative Western blot (left) or immunofluorescence (right). (FIG. 9B) Representative cell cycle analysis by flow cytometry of proliferating parental (KPr) tumor cells or cells after p53 induction (KPr^{p53}). (FIG. 9C) β-gal staining of proliferating (KPr^{p53}P) and arrested (KPr^{p53}A) cells. Mean±SEM of 3 experiments is shown (top). Bottom—typical staining. P values were calculated in two-sided Student's t-test. (FIG. 9D) Western blot of markers associated with senescence. Representative examples of three independent experiments. (FIG. 9E) Western blot of p21 staining in KPr^{p53}A cells. Representative examples of three independent experiments. (FIG. 9F) Representative staining of H3K9Me. Scale bars=50 μm.

FIG. 10A-FIG. 10E show characteristics of tumor growth in vivo. (FIG. 10A) Representative bioluminescence images of mice injected with 2.5×10⁴ proliferating (KPr^{p53}P) or arrested (KPr^{p53}A) cells at indicated time after the injection. (FIG. 10B) Left: H&E staining of lungs harvested from mice. Large tumor areas are readily visible. Scale bar 50 μm. Right: Quantification of luciferase signal in mouse lung area by IVIS two weeks after injection of KPr^{p53}P cells. Each dot represents a different mouse. P value is calculated in two-sided Student's t-test. (FIG. 10C) Representative images of tumor cells in lungs and liver of NOD/SCID mice. GFP-positive KPr^{p53}A cells were detected in 5 μm thick frozen sections of lung or liver. Green—GFP-positive tumor cells; blue—nuclei stained with Hoechst 333342. Representative images are shown. Scale bar is 50 μm. (FIG. 10D) Percentage of PMN in spleens of C57BL/6 or NOD/SCID LLC-bearing mice compared to tumor-free mice (naive). Mean±SEM and results of individual mice are shown. (FIG. 10E) Fold increase in the number of KPr^{p53}A cells in the presence of PMN isolated from NOD/SCID tumor-bearing mice (TBM) over KPr^{p53}A cells cultured alone.

FIG. 11A-FIG. 11G show the mechanism of dormant tumor cell reactivation. (FIG. 11A) Fold increase in the number of KPr^{p53}A cells cultured with PMN and S100A8/A9, thapsigargin (Tha), or Phorbol myristate acetate (PMA) over KPr^{p53}A cells alone (ctrl). Mean±SEM of 3 independent experiments with 16 replicates for each are shown. (FIG. 11B) Fold increase in the number of KPr^{p53}A cells cultured with monocytes in the presence of S100A8/A9 and indicated cytokines. Mean±SEM of 10 independent experiments with 16 replicates each are shown. (FIG. 11C) Neutrophil extracellular traps extruded by PMN or PMN-MDSC alone or in culture with recombinant S100A8/A9. Positive controls are PMN or PMN-MDSC cultured in presence of PMA. Mean±SEM of 3 independent experiments are shown. (FIG. 11D) Fold increase in KPr^{p53}A cell count in the presence of PAD4 KO PMN and S100A8/A9 or NE over KPr^{p53}A cells alone (ctrl). NE alone or S100a9 alone were used as control. Mean±SEM of 3 independent experiments with 16 replicates for conditions. P values were calculated by ANOVA test with correction for multiple comparisons. (FIG. 11E) qRT-PCR of NE receptor ADAR-20 on PMN, arrested (KPr^{p53}A), or proliferating (KPr^{p53}P) tumor cells. Mean±SEM of an independent experiment performed in triplicates are shown. (FIG. 11F) Growth of KPr cells in the presence of different concentration of norepinephrine (NE). Mean±SEM are reported for each time point (n=3). (FIG. 11G) Proportion of live PMN after exposure to norepinephrine (NE) or the O-blocker ICI-118,551 at the indicated concentration. Bars represent at least 3 different biological replicates. Mean±SEM are reported.

FIG. 12A-FIG. 12F show characterization of cisplatin-induced tumor cell dormancy. (FIG. 12A) Gating strategy to sort arrested (A) and proliferating (P) cells from cisplatin-exposed tumor cells. (FIG. 12B) Representative cell cycle analysis by flow cytometry of proliferating (LL2, A549, OVCAR3) or arrested (LL2^{cis}A, A549^{cis}A, OVCAR3^{cis}A) tumor cells. (FIG. 12C) Western blot analysis of markers associated with senescence on tumor cells. (FIG. 12D) Western blot analysis of p21 in cisplatin-treated cells. (FIG. 12E) R-gal staining of proliferating (LL2, A549, OVCAR3) or arrested (LL2^{cis}A, A549^{cis}A, OVCAR3^{cis}A) cells. (FIG. 12F) H3K9Me staining of indicated cells. Scale bars=50 μm. Representative pictures and quantification with mean±SEM of 3 different experiments are shown. P values were calculated with a two-sided Student's t-test.

FIG. 13A-FIG. 13E show characterization of stress induction model. (FIG. 13A) Experimental design. (FIG. 13B) Norepinephrine (NE) in plasma of NOD/SCID mice undergoing stress (Stress) or control mice (no Stress) at the indicated time points as well as in plasma of LLC tumor-bearing mice (measured by ELISA). Results of independent experiments with 3-4 mice with stress and 7 tumor-bearing mice. Mean±SEM are shown. P values were calculated by ANOVA test with correction for multiple comparisons. (FIG. 13C) Survival of mice with indicated treatment based on humane end-points for the termination of the experiments; n=10 for each group. P values were calculated in Log-rank (Mantel-Cox) test. (FIG. 13D) Flow cytometry of PMN infiltration of lung (upper) or spleen (lower) of C57BL/6 mice undergoing stress. Each dot represents a single mouse, and mean±SEM are shown. P values are calculated by two-sided Student's t-test. (FIG. 13E) Representative images showing detection of PMN in 5-μm thick frozen sections of lungs after staining with anti-Ly6G-AlexaFluor594 antibody (lighter staining). Nuclei are stained with Hoechst 333342 (darker staining). Scale bar is 50 μm.

FIG. 14A-FIG. 14B show the effect of S100A9 on PMN activity. (FIG. 14A) DCFDA measured by flow cytometry in PMN treated with S100A8/A9 or NE. Each dot represents an individual mouse. Mean±SEM are shown. (FIG. 14B) qRT-PCR of Ptges, Ptgs2, and Arg1 expression in mouse PMN treated with S100A8/A9 or NE. Each dot represents an individual mouse. Mean±SEM are shown.

FIG. 15A-FIG. 15F show characterization of lipids extracted from PMN. (FIG. 15A) LC-ESI-MS/MS mass spectrometry of PMN. Typical spectra of phosphatidylcholine (PC, left panel) and phosphatidylethanolamine (PE, right panel) are shown. (FIG. 15B) Phosphatidylethanolamine (PE) lipids before and after peroxidation leading to formation of 4-hydroxynonenal (4-HNE) Michael adducts. (FIG. 15C) Schema illustrating the reaction of PE plasmalogen with MPO leading to production of Lyso-PE. (FIG. 15D) Measurement of oxidatively truncated PC species after incubation of PMN with S100A8/A9. (FIG. 15E) Heat map of unsaturated lyso-PE from naïve PMN, PMN exposed to S100A8/A9 and PMN isolated from stressed mice. (FIG. 15F) Representative spectra of different species of synthetic phosphatidylethanolamine (PE 18:0p/20:4; 18:0p/22:6; 18:0/20:4) untreated (top) and treated with MPO/H2O2/NaCl with generation of oxidized lipid species (middle) and lyso-PE (LPE, bottom).

FIG. 16A-FIG. 16D show characterization of reactivated tumor cells. (FIG. 16A) Growth of KPr and KPr^{p53}A^{react} cells in vitro. Mean±SEM of 3 independent experiments with 3 biological replicates are shown. (FIG. 16B) Number of significantly changed genes found in KPr and KPr^{p53}A^{react} cells as measured by RNA sequencing. (FIG. 16C) Pathways associated with FGFR1/2 upregulation from RNASeq data. (FIG. 16D) Effect of FGFR inhibitor on tumor cell proliferation. Luminescence count (proportional to number of living cells in culture) of indicated cell types untreated or treated with indicated doses of FGFR inhibitor BGJ398. Mean±SEM and results of each independent experiment with 3 replicates are shown. P values were calculated using ANOVA test with corrections for multiple comparisons.

### DETAILED DESCRIPTION OF THE INVENTION

Tumor recurrence years after seemingly successful treatment of primary tumors is one of the major causes of mortality in cancer patients. Reactivation of dormant tumor cells is largely responsible for this phenomenon. Using models of lung and ovarian cancer, a specific mechanism that governs this process mediated by stress and neutrophils is described herein.

While the role of adrenergic receptors (AR), especially β-AR, have been associated with various malignancies, the mechanism linking stress, β-AR, and reactivation of dormant cancer cells has not been previously elucidated. As described herein, stress hormones cause rapid release of S100A8/A9 proteins by neutrophils. S100A8/A9 induce activation of myeloperoxidase (MPO) resulting in accumulation of oxidized lipids. These lipids up-regulate the fibroblast growth factor (FGFR) pathway in tumor cells causing tumor cells to leave dormancy and form tumor lesions. Higher serum levels of S100A8/A9 were associated with shorter time to recurrence in patients with lung cancer after complete tumor resection. Targeting of S100A8/A9 or β2 adrenergic receptors abrogated stress induced reactivation of dormant tumor cells. These observations demonstrate a mechanism linking stress, and specific neutrophil activation with early recurrence in cancer.

It is to be noted that the term “a” or “an” refers to one or more. As such, the terms “a” (or “an”), “one or more,” and “at least one” are used interchangeably herein.

While various embodiments in the specification are presented using “comprising” language, under other circumstances, a related embodiment is also intended to be interpreted and described using “consisting of” or “consisting essentially of” language. The words “comprise”, “comprises”, and “comprising” are to be interpreted inclusively rather than exclusively. The words “consist”, “consisting”, and its variants, are to be interpreted exclusively, rather than inclusively.

As used herein, the term “about” means a variability of 10% from the reference given, unless otherwise specified.

“Upregulate” and “upregulation”, as used herein, refer to an elevation in the level of expression of a product of one or more genes in a cell or the cells of a tissue or organ.

“Inhibit” or “downregulate”, as used herein refer to a reduction in the level of expression of a product of one or more genes in a cell or the cells of a tissue or organ.

By the general terms “blocker”, “inhibitor”, or “antagonist” is meant an agent that inhibits, either partially or fully, the activity or production of a target molecule, e.g., as used herein, e.g., S100A8/A9. In particular, these terms refer to a composition or compound or agent capable of decreasing levels of gene expression, mRNA levels, protein levels or protein activity of the target molecule. Illustrative forms of antagonists include, for example, proteins, polypeptides, peptides (such as cyclic peptides), antibodies or antibody fragments, peptide mimetics, nucleic acid molecules, antisense molecules, ribozymes, aptamers, RNAi molecules, and small organic molecules. Illustrative non-limiting mechanisms of antagonist inhibition include repression of ligand synthesis and/or stability (e.g., using, antisense, ribozymes or RNAi compositions targeting the ligand gene/nucleic acid), blocking of binding of the ligand to its cognate receptor (e.g., using anti-ligand aptamers, antibodies or a soluble, decoy cognate receptor), repression of receptor synthesis and/or stability (e.g., using, antisense, ribozymes or RNAi compositions targeting the ligand receptor gene/nucleic acid), blocking of the binding of the receptor to its cognate receptor (e.g., using receptor antibodies) and blocking of the activation of the receptor by its cognate ligand (e.g., using receptor tyrosine kinase inhibitors). In addition, the blocker or inhibitor may directly or indirectly inhibit the target molecule.

The terms “RNA interference,” “RNAi,” “miRNA,” and “siRNA” refer to any method by which expression of a gene or gene product is decreased by introducing into a target cell one or more double-stranded RNAs, which are homologous to a gene of interest (particularly to the messenger RNA of the gene of interest). Gene therapy, i.e., the manipulation of RNA or DNA using recombinant technology and/or treating disease by introducing modified RNA or modified DNA into cells via a number of widely known and experimental vectors, recombinant viruses and CRISPR technologies, may also be employed in delivering, via modified RNA or modified DNA, effective inhibition of S100A8/A9 pathways and gene products and R adrenergic pathways and gene products to accomplish the outcomes described herein with the combination therapies described. Such genetic manipulation can also employ gene editing techniques such as CRISPR (Clustered Regularly Interspaced Short Palindromic Repeats) and TALEN (transcription activator-like effector genome modification), among others. See, for example, the textbook National Academies of Sciences, Engineering, and Medicine. 2017. Human Genome Editing: Science, Ethics, and Governance. Washington, D.C.: The National Academies Press. https://doi.org/10.17226/24623, incorporated by reference herein for details of such methods.

A “subject” is a mammal, e.g., a human, mouse, rat, guinea pig, dog, cat, horse, cow, pig, or non-human primate, such as a monkey, chimpanzee, baboon or gorilla. The term “patient” may be used interchangeably with the term subject. In one embodiment, the subject is a human. The subject may be of any age, as determined by the health care provider. In certain embodiments described herein, the patient is a subject who has previously been diagnosed with cancer. The subject may have been treated for cancer previously, or is currently being treated for cancer. In one embodiment, the subject is experiencing stress which has an impact on the beta-adrenergic signaling pathway.

“Sample” as used herein means any biological fluid or tissue that contains blood cells, immune cells and/or cancer cells. In one embodiment, the sample is whole blood. In another embodiment, the sample is plasma. Other useful biological samples include, without limitation, peripheral blood mononuclear cells, plasma, saliva, urine, synovial fluid, bone marrow, cerebrospinal fluid, vaginal mucus, cervical mucus, nasal secretions, sputum, semen, amniotic fluid, bronchoscopy sample, bronchoalveolar lavage fluid, and other cellular exudates from a patient having cancer. Such samples may further be diluted with saline, buffer or a physiologically acceptable diluent. Alternatively, such samples are concentrated by conventional means.

The term “cancer” or “proliferative disease” as used herein means any disease, condition, trait, genotype, or phenotype characterized by unregulated cell growth or replication as is known in the art. A “cancer cell” is cell that divides and reproduces abnormally with uncontrolled growth. This cell can break away from the site of its origin (e.g., a tumor) and travel to other parts of the body and set up another site (e.g., another tumor), in a process referred to as metastasis. A “tumor” is an abnormal mass of tissue that results from excessive cell division that is uncontrolled and progressive and is also referred to as a neoplasm. Tumors can be either benign (not cancerous) or malignant. The methods described herein are useful for the treatment of cancer and tumor cells, i.e., both malignant and benign tumors. In various embodiments of the methods and compositions described herein, the cancer can include, without limitation, breast cancer, lung cancer, prostate cancer, colorectal cancer, brain cancer, esophageal cancer, stomach cancer, bladder cancer, pancreatic cancer, cervical cancer, head and neck cancer, ovarian cancer, melanoma, acute and chronic lymphocytic and myelocytic leukemia, myeloma, Hodgkin's and non-Hodgkin's lymphoma, and multi-drug resistant cancers. In one embodiment, the cancer is lung cancer. In another embodiment, the cancer is ovarian cancer.

“Control” or “control level” as used herein refers to the source of the reference value for S100A8/A9 levels as well as the particular panel of control subjects identified in the examples below. In some embodiments, the control subject is a healthy subject with no disease. In another embodiment, the control subject is a patient who has been successfully treated for cancer. In yet other embodiments, the control or reference is the same subject from an earlier time point. Selection of the particular class of controls depends upon the use to which the diagnostic/monitoring methods and compositions are to be put by the physician.

The terms “analog”, “modification”, and “derivative” refer to biologically active derivatives of the reference molecule that retain desired activity as described herein. Preferably, the analog, modification or derivative has at least the same desired activity as the native molecule, although not necessarily at the same level. The terms also encompass purposeful mutations that are made to the reference molecule.

By “fragment” is intended a molecule consisting of only a part of the intact full-length polypeptide sequence and structure. The fragment can include a C terminal deletion, an N terminal deletion, and/or an internal deletion of the native polypeptide. A fragment will generally include at least about 5-10 contiguous amino acid residues of the full length molecule, preferably at least about 15-25 contiguous amino acid residues of the full length molecule, and most preferably at least about 20 50 or more contiguous amino acid residues of the full length molecule, or any integer between 5 amino acids and the full length sequence, provided that the fragment in question retains the ability to elicit the desired biological response, although not necessarily at the same level.

By the term “antibody” or “antibody molecule” is any immunoglobulin, including antibodies and fragments thereof, that binds to a specific antigen. As used herein, antibody or antibody molecule contemplates intact immunoglobulin molecules, immunologically active portions of an immunoglobulin molecule, and fusions of immunologically active portions of an immunoglobulin molecule.

The antibody may be a naturally occurring antibody or may be a synthetic or modified antibody (e.g., a recombinantly generated antibody; a chimeric antibody; a bispecific antibody; a humanized antibody; a camelid antibody; and the like). The antibody may comprise at least one purification tag. In a particular embodiment, the framework antibody is an antibody fragment. The term “antibody fragment” includes a portion of an antibody that is an antigen binding fragment or single chains thereof. An antibody fragment can be a synthetically or genetically engineered polypeptide. Examples of binding fragments encompassed within the term “antigen-binding portion” of an antibody include (i) a Fab fragment, a monovalent fragment consisting of the VL, VH, CL and CH1 domains; (ii) a F(ab′)2 fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb fragment, which consists of a VH domain; and (vi) an isolated complementarity determining region (CDR). Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules (known as single chain Fv (scFv). Such single chain antibodies are also intended to be encompassed within the term “antigen-binding fragment” of an antibody. These antibody fragments are obtained using conventional techniques known to those in the art, and the fragments can be screened for utility in the same manner as whole antibodies. Antibody fragments include, without limitation, immunoglobulin fragments including, without limitation: single domain (Dab; e.g., single variable light or heavy chain domain), Fab, Fab′, F(ab′)2, and F(v); and fusions (e.g., via a linker) of these immunoglobulin fragments including, without limitation: scFv, scFv2, scFv-Fc, minibody, diabody, triabody, and tetrabody. The antibody may also be a protein (e.g., a fusion protein) comprising at least one antibody or antibody fragment.

The term “derived from” is used to identify the original source of a molecule (e.g., bovine or human) but is not meant to limit the method by which the molecule is made which can be, for example, by chemical synthesis or recombinant means.

As used herein, the term “a therapeutically effective amount” refers an amount sufficient to achieve the intended purpose. For example, an effective amount of an S100A8/A9 inhibitor is sufficient to inhibit dormant cancer cells from returning to a proliferative state. An effective amount for treating or ameliorating a disorder, disease, or medical condition is an amount sufficient to result in a reduction or complete removal of the symptoms of the disorder, disease, or medical condition. The effective amount of a given therapeutic agent will vary with factors such as the nature of the agent, the route of administration, the size and species of the animal to receive the therapeutic agent, and the purpose of the administration. The effective amount in each individual case may be determined by a skilled artisan according to established methods in the art.

The term “carrier” refers to a diluent, adjuvant, excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water is a preferred carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents. These compositions can take the form of solutions, suspensions, emulsion, tablets, pills, capsules, powders, sustained-release formulations, and the like. The composition can be formulated as a suppository, with traditional binders and carriers such as triglycerides. Oral formulation can include standard carriers such as pharmaceutical grades of mannitol, lactose, starch, magnesium stearate, sodium saccharine, cellulose, magnesium carbonate, etc. Examples of suitable pharmaceutical carriers are described in Remington's Pharmaceutical Sciences, 18th Ed., Gennaro, ed. (Mack Publishing Co., 1990). The formulation should suit the mode of administration.

Routes of administration include, but are not limited to, intradermal, intramuscular, intraperitoneal, intravenous, subcutaneous, intranasal, epidural, and oral routes. The agent may be administered by any convenient route, for example by infusion or bolus injection, by absorption through epithelial or mucocutaneous linings (e.g., oral mucosa, rectal and intestinal mucosa, etc.) and may be administered together with other biologically active agents. Administration can be systemic or local.

As used herein, “disease”, “disorder”, and “condition” are used interchangeably, to indicate an abnormal state in a subject.

Provided herein, in one aspect, are methods of inhibiting reactivation of dormant tumor cells in a subject. As described herein, targeting of S100A8/A9 or 02 adrenergic receptors abrogates stress induced reactivation of dormant tumor cells. Thus, provided herein, are methods of inhibiting the recurrence of cancer in subject associated with stress-induced β-adrenergic pathway signaling.

### S100A8/A9

In one embodiment, the method includes inhibiting or reducing S100A8/A9 in the subject. S100A8/A9, also known as calprotectin or MRP8/14, is a heterocomplex of the two S100 calcium binding proteins, S100A8 (calgranulin A or MRP8—myeloid related protein 8) and S100A9 (calgranulin B or MRP14—myeloid related protein 14). S100A8 and S100A9 are secreted as a heterodimeric complex (S100A8/A9), called calprotectin, from neutrophils and monocytes/macrophages. S100A8 has a molecular weight of 11.0 kDa and S100A9 exists in two forms, 13.3 kDa and truncated 12.9 kDa. Both proteins are similar to other members of the S100 family in that they contain two EF-hand motifs that bind calcium ions. Ca2+-binding induces the formation of heterocomplexes S100A8/S100A9 and (S100A8)2/(S100A9)2 homocomplexes.

In one embodiment, the method includes administering an effective amount of an inhibitor of S100A8. In another embodiment, the method includes administering an effective amount of an inhibitor of S100A9. In yet another embodiment, the method includes administering an effective amount of an inhibitor of S100A8/A9. As used herein, wherein a reference is made to an inhibitor of S100A8/A9, it is meant to refer to an inhibitor of S100A8, an inhibitor of S100A9, or an inhibitor of S100A8/A9. Inhibitors of S100A8/A9 are known in the art. Inhibitors encompassed herein include those that target S100A8 and homodimers thereof; S100A9 and homodimers thereof; and S100A8/A9 heterodimers thereof. Such inhibitors include, without limitation, tasquinimod, paquinimod, bromodomain inhibitor-JQ1, and arachidonic acid. In one embodiment, the inhibitor is tasquinimod. In another embodiment, the inhibitor is paquinimod. In yet another embodiment, the inhibitor is bromodomain inhibitor-JQ1. In another embodiment, the inhibitor is arachidonic acid.

In one embodiment, the effective amount of the S100A8/A9 inhibitor is an amount ranging from about 0.01 mg/ml to about 10 mg/ml, including all amounts therebetween and end points. In one embodiment, the effective amount of the S100A8/A9 inhibitor is about 0.1 mg/ml to about 5 mg/ml, including all amounts therebetween and end points. In another embodiment, the effective amount of the S100A8/A9 inhibitor is about 0.3 mg/ml to about 1.0 mg/ml, including all amounts therebetween and end points. In another embodiment, the effective amount of the S100A8/A9 inhibitor is about 0.3 mg/ml. In another embodiment, the effective amount of the S100A8/A9 inhibitor is about 0.4 mg/ml. In another embodiment, the effective amount of the S100A8/A9 inhibitor is about 0.5 mg/ml. In another embodiment, the effective amount of the S100A8/A9 inhibitor is about 0.6 mg/ml. In another embodiment, the effective amount of the S100A8/A9 inhibitor is about 0.7 mg/ml. In another embodiment, the effective amount of the S100A8/A9 inhibitor is about 0.8 mg/ml. In another embodiment, the effective amount of the S100A8/A9 inhibitor is about 0.9 mg/ml. In another embodiment, the effective amount of the S100A8/A9 inhibitor is about 1.0 mg/ml.

In one embodiment, the effective amount of the S100A8/A9 inhibitor is an amount ranging from about 1 μM to about 2 mM, including all amounts therebetween and end points. In one embodiment, the effective amount of the S100A8/A9 inhibitor is about 10 μM to about 100 μM, including all amounts therebetween and end points. In another embodiment, the effective amount of the S100A8/A9 inhibitor is about 5 μM. In another embodiment, the effective amount of the S100A8/A9 inhibitor is about 10 μM. In another embodiment, the effective amount of the S100A8/A9 inhibitor is about 20 μM. In another embodiment, the effective amount of the S100A8/A9 inhibitor is about 50 μM. In another embodiment, the effective amount of the S100A8/A9 inhibitor is about 100 μM. In another embodiment, the effective amount of the S100A8/A9 inhibitor is about 200 μM. In another embodiment, the effective amount of the S100A8/A9 inhibitor is about 300 μM. In another embodiment, the effective amount of the S100A8/A9 inhibitor is about 400 μM. In another embodiment, the effective amount of the S100A8/A9 inhibitor is about 500 μM. In another embodiment, the effective amount of the S100A8/A9 inhibitor is about 600 μM. In another embodiment, the effective amount of the S100A8/A9 inhibitor is about 700 μM. In another embodiment, the effective amount of the S100A8/A9 inhibitor is about 800 μM. In another embodiment, the effective amount of the S100A8/A9 inhibitor is about 900 μM. In another embodiment, the effective amount of the S100A8/A9 inhibitor is about 1 mm. In another embodiment, the effective amount of the S100A8/A9 inhibitor is about 1.25 mM. In another embodiment, the effective amount of the S100A8/A9 inhibitor is about 1.5 mM. In another embodiment, the effective amount of the S100A8/A9 inhibitor is about 1.75 mM. In another embodiment, the effective amount of the S100A8/A9 inhibitor is about 2 mM.

In certain embodiments, inhibiting or reducing S100A8/A9 in the subject can be accomplished by reducing the amount of mRNA, e.g., via RNAi, or protein in the subject. Thus, in one embodiment, S100A8/A9 is downregulated by reducing the level of S100A8 or S100A9 mRNA in the subject. S100A8 or S100A9 mRNA levels may be reduced, in one embodiment, using siRNA. siRNA can be generated against S100A8, S100A9, or S100A8/A9 using sequences known in the art. For example, the following S100A8/A9 sequences can be found in GenBank: NM_001319197.1, NM_001319198.1, NM_001319201.1, and NM_002964.4 (S100A8) and NM_002965.3 (S100A9) each of which is incorporated herein by reference. Exemplary sequences are provided.

S100A8 -
SEQ ID NO: 1
MLTELEKALN SIIDVYHKYS LIKGNFHAVY RDDLKKLLET
ECPQYIRKKG ADVWFKELDI NTDGAVNFQE FLILVIKMGV
AAHKKSHEES HKE
S100A8 -
SEQ ID NO: 2
atgtc tcttgtcagc tgtctttcag aagacctggt tctgtttttc
aggtggggca agtccgtggg catcatgttg accgagctgg
agaaagcctt gaactctatc atcgacgtct accacaagta
ctccctgata aaggggaatt tccatgccgt ctacagggat
gacctgaaga aattgctaga gaccgagtgt cctcagtata
tcaggaaaaa gggtgcagac gtctggttca aagagttgga
tatcaacact gatggtgcag ttaacttcca ggagttcctc
attctggtga taaagatggg cgtggcagcc cacaaaaaaa
gccatgaaga aagccacaaa gagtagc.
S100A9 -
SEQ ID NO: 3
MTCKMSQLER NIETIINTFH QYSVKLGHPD TLNQGEFKEL
VRKDLQNFLK KENKNEKVIE HIMEDLDTNA DKQLSFEEFI
MLMARLTWAS HEKMHEGDEG PGHHHKPGLG EGTP
S100A9 -
SEQ ID NO: 4
atgactt gcaaaatgtc gcagctggaa cgcaacatag
agaccatcat caacaccttc caccaatact ctgtgaagct
ggggcaccca gacaccctga accaggggga attcaaagag
ctggtgcgaa aagatctgca aaattttctc aagaaggaga
ataagaatga aaaggtcata gaacacatca tggaggacct
ggacacaaat gcagacaagc agctgagctt cgaggagttc
atcatgctga tggcgaggct aacctgggcc tcccacgaga
agatgcacga gggtgacgag ggccctggcc accaccataa
gccaggcctc ggggagggca ccccctaa

In certain embodiments, inhibiting or reducing S100A8/A9 in the subject can be accomplished by reducing the amount of S100A8/A9 protein in the subject via administration of an antibody that neutralizes or blocks the action of S100A8/A9 (e.g., an anti-S100A8/A9 antibody). An anti-S100A8/A9 antibody can be an antibody that binds to, blocks, competes or interferes with binding or activity of, any of the components of S100A8/A9, or the heterocomplex itself.

### FGFR

Provided herein, in another aspect, are methods of inhibiting reactivation of dormant tumor cells in a subject by inhibiting or reducing a fibroblast growth factor receptor (FGFR) in the subject.

Fibroblast growth factor receptors (FGFRs) are a subgroup of the family of tyrosine kinase receptors. They consist of an extracellular domain (glycoside acidic box, immunoglobulin-like domain, and calmodulin-like domain), a transmembrane domain, and an intracellular tyrosine kinase domain. Its active form is the dimer that provokes phosphorylation of the tyrosine intracellular endings. This promotes activation of intracellular events that lead to Ca2+ release, protein kinase C activation, and kinase phosphorylation that ends with activation of transcription factors. FGFR1, FGFR2, and FGFR3 interact in the cell-to-cell signaling process. They have complex functions involving proliferation, the end of the cellular cycle, cellular migration, differentiation, and apoptosis. FGFR2 promotes proliferation, and FGFR1 acts in the differentiation of cranial sutures. A mutation in any of these genes promotes lengthening of the signal, which causes early maturation of bone cells in the developing embryo and premature fusion of sutures, hands, and feet. FGFR3 is an inhibitor of proliferation during chondrogenesis.

In one embodiment, the method includes administering an effective amount of an inhibitor of FGFR1. In another embodiment, the method includes administering an effective amount of an inhibitor of FGFR2. In yet another embodiment, the method includes administering an effective amount of an inhibitor of FGFR3. As used herein, wherein a reference is made to an inhibitor of FGFR, it is meant to refer to an inhibitor of FGFR1, an inhibitor of FGFR2, an inhibitor of FGFR3, a pan-FGFR inhibitor, or an inhibitor of FGF7.

Inhibitors of FGFR are known in the art. Such inhibitors include, without limitation, infigratinib phosphate, erdafitinib, deranzantinib hydrochloride, rogaratinib, HMPL-453, futibatinib, PRN-1371, LY-2874455, BPI-17213, CPL-043, and ASP-5878. In one embodiment, the FGFR inhibitor is (BGJ398). In another embodiment, the FGFR inhibitor is erdafitinib (e.g., Balversa, Janssen Pharmaceutical). In another embodiment, the FGFR inhibitor is pemigatinib (e.g., Pemazyre, Incyte Corp.). In another embodiment, the FGFR inhibitor is deranzantinib hydrochloride (e.g., BAL087, Basilea Pharmaceutica Ltd.). In another embodiment, the FGFR inhibitor is rogaratinib (e.g., Bayer Pharmaceutical). In another embodiment, the FGFR inhibitor is HMPL-453. In another embodiment, the FGFR inhibitor is futibatinib (e.g., TAS-120, Taiho Oncology). In another embodiment, the FGFR inhibitor is PRN-1371. In another embodiment, the FGFR inhibitor is LY-2874455. In another embodiment, the FGFR inhibitor is BPI-17213. In another embodiment, the FGFR inhibitor is CPL-043. In another embodiment, the FGFR inhibitor is ASP-5878. In another embodiment, the FGFR inhibitor is DEBIO 1347 (Debiopharm International SA). In another embodiment, the FGFR inhibitor is ICP-192 (InnoCare Pharma Limited). In another embodiment, the FGFR inhibitor is infigratinib (e.g., BGJ398, QED Therapeutics). In another embodiment, the FGFR inhibitor is AZD4547 (AstraZeneca). In another embodiment, the FGFR inhibitor is PRN1371 (Principia Biopharma). In another embodiment, the FGFR inhibitor is sulfatinib (Hutchison MediPharma). In another embodiment, the FGFR inhibitor is B-701 (BioClin Therapeutics 2L). In another embodiment, the FGFR inhibitor is INCB54828 (Incyte Corp.).

In one embodiment, the effective amount of the FGFR inhibitor is an amount ranging from about 0.01 mg/ml to about 10 mg/ml, including all amounts therebetween and end points. In one embodiment, the effective amount of the FGFR inhibitor is about 0.1 mg/ml to about 5 mg/ml, including all amounts therebetween and end points. In another embodiment, the effective amount of the FGFR inhibitor is about 0.3 mg/ml to about 1.0 mg/ml, including all amounts therebetween and end points. In another embodiment, the effective amount of the FGFR inhibitor is about 0.3 mg/ml. In another embodiment, the effective amount of the FGFR inhibitor is about 0.4 mg/ml. In another embodiment, the effective amount of the FGFR inhibitor is about 0.5 mg/ml. In another embodiment, the effective amount of the FGFR inhibitor is about 0.6 mg/ml. In another embodiment, the effective amount of the FGFR inhibitor is about 0.7 mg/ml. In another embodiment, the effective amount of the FGFR inhibitor is about 0.8 mg/ml. In another embodiment, the effective amount of the FGFR inhibitor is about 0.9 mg/ml. In another embodiment, the effective amount of the FGFR inhibitor is about 1.0 mg/ml.

In one embodiment, the effective amount of the FGFR inhibitor is an amount ranging from about 1 μM to about 2 mM, including all amounts therebetween and end points. In one embodiment, the effective amount of the FGFR inhibitor is about 10 μM to about 100 μM, including all amounts therebetween and end points. In another embodiment, the effective amount of the FGFR inhibitor is about 5 μM. In another embodiment, the effective amount of the FGFR inhibitor is about 10 μM. In another embodiment, the effective amount of the FGFR inhibitor is about 20 μM. In another embodiment, the effective amount of the FGFR inhibitor is about 50 μM. In another embodiment, the effective amount of the FGFR inhibitor is about 100 μM. In another embodiment, the effective amount of the FGFR inhibitor is about 200 μM. In another embodiment, the effective amount of the FGFR inhibitor is about 300 μM. In another embodiment, the effective amount of the FGFR inhibitor is about 400 μM. In another embodiment, the effective amount of the FGFR inhibitor is about 500 μM. In another embodiment, the effective amount of the FGFR inhibitor is about 600 μM. In another embodiment, the effective amount of the FGFR inhibitor is about 700 μM. In another embodiment, the effective amount of the FGFR inhibitor is about 800 μM. In another embodiment, the effective amount of the FGFR inhibitor is about 900 μM. In another embodiment, the effective amount of the FGFR inhibitor is about 1 mM. In another embodiment, the effective amount of the FGFR inhibitor is about 1.25 mM. In another embodiment, the effective amount of the FGFR inhibitor is about 1.5 mM. In another embodiment, the effective amount of the FGFR inhibitor is about 1.75 mM. In another embodiment, the effective amount of the FGFR inhibitor is about 2 mM.

In one embodiment, FGFR is downregulated by reducing the level of FGFR mRNA in the subject. In certain embodiments, inhibiting or reducing FGFR in the subject can be accomplished by reducing the amount of mRNA, e.g., via RNAi, or protein in the subject.

FGFR mRNA levels may be reduced, in one embodiment, using siRNA. SiRNA can be generated against FGFR using sequences known in the art. For example, the following FGFR sequences can be found in GenBank:

### FGFR1:

### NP_001167534.1, NM_001174063.1 [P11362-2]

### NP_001167535.1, NM_001174064.1 [P11362-20]

### NP_001167536.1, NM_001174065.1 [P11362-7]

### NP_001167537.1, NM_001174066.1 [P11362-3]

### NP_001167538.1, NM_001174067.1 [P11362-21]

### NP_056934.2, NM_015850.3 [P11362-7]

### NP_075593.1, NM 023105.2 [P11362-3]

### NP_075594.1, NM 023106.2 [P11362-14]

### NP_075598.2, NM 023110.2 [P11362-1]

### FGFR2:

### NP_000132.3, NM_000141.4 [P21802-1]

### NP_001138385.1, NM_001144913.1 [P21802-17]

### NP_001138386.1, NM_001144914.1 [P21802-23]

### NP_001138387.1, NM_001144915.1 [P21802-21]

### NP_001138388.1, NM_001144916.1

### NP_001138389.1, NM_001144917.1 [P21802-15]

### NP_001138390.1, NM_001144918.1 [P21802-20]

### NP_001138391.1, NM_001144919.1 [P21802-22]

### NP_001307583.1, NM_001320654.1

### NP_001307587.1, NM_001320658.1 [P21802-5]

### NP_075259.4, NM_022970.3 [P21802-3]

### NP_075418.1, NM 023029.2

### FGF7:

### NP_002000.1, NM 002009.3 [P21781-1]

Each of these sequences is incorporated herein by reference.

In certain embodiments, inhibiting or reducing FGFR in the subject can be accomplished by reducing the amount of FGFR protein in the subject via administration of an antibody that neutralizes or blocks the action of FGFR (e.g., an anti-FGFR antibody). An anti-FGFR antibody can be an antibody that binds to, blocks, competes or interferes with binding or activity of, any FGFR, including FGFR1, FGFR2, and FGF7.

### MPO

Provided herein, in another aspect, are methods of inhibiting reactivation of dormant tumor cells in a subject by inhibiting or reducing myeloperoxidase (MPO) in the subject.

MPO is part of the host defense system of polymorphonuclear leukocytes. It is responsible for microbicidal activity against a wide range of organisms. In the stimulated PMN, MPO catalyzes the production of hypohalous acids, primarily hypochlorous acid in physiologic situations, and other toxic intermediates that greatly enhance PMN microbicidal activity.

In one embodiment, the method includes administering an effective amount of an inhibitor of MPO. Inhibitors of MPO are known in the art. Such inhibitors include, without limitation benzoic acid hydrazides including 4-aminobenzoic acid hydrazide, 2-thioxanthines, paracetamol, isoniazid, salicylhydroxamicacid (SHA), hydroxamic acids [RCNOHOH or RC(O)NHOH], serotonin, melatonin, 5-fluorotryptamine and 5-chlorotryptamine, and flavonoids including quercetin, resveratrol etc. See, e.g., Tamara Lazarevic-Pasti, et al, Myeloperoxidase Inhibitors as Potential Drugs, Current Drug Metabolism, 2015, 16, 168-190.

In one embodiment, the effective amount of the MPO inhibitor is an amount ranging from about 0.01 mg/ml to about 10 mg/ml, including all amounts therebetween and end points. In one embodiment, the effective amount of the MPO inhibitor is about 0.1 mg/ml to about 5 mg/ml, including all amounts therebetween and end points. In another embodiment, the effective amount of the MPO inhibitor is about 0.3 mg/ml to about 1.0 mg/ml, including all amounts therebetween and end points. In another embodiment, the effective amount of the MPO inhibitor is about 0.3 mg/ml. In another embodiment, the effective amount of the MPO inhibitor is about 0.4 mg/ml. In another embodiment, the effective amount of the MPO inhibitor is about 0.5 mg/ml. In another embodiment, the effective amount of the MPO inhibitor is about 0.6 mg/ml. In another embodiment, the effective amount of the MPO inhibitor is about 0.7 mg/ml. In another embodiment, the effective amount of the MPO inhibitor is about 0.8 mg/ml. In another embodiment, the effective amount of the MPO inhibitor is about 0.9 mg/ml. In another embodiment, the effective amount of the MPO inhibitor is about 1.0 mg/ml.

In one embodiment, the effective amount of the MPO inhibitor is an amount ranging from about 1 μM to about 2 mM, including all amounts therebetween and end points. In one embodiment, the effective amount of the MPO inhibitor is about 10 μM to about 100 μM, including all amounts therebetween and end points. In another embodiment, the effective amount of the MPO inhibitor is about 5 μM. In another embodiment, the effective amount of the MPO inhibitor is about 10 μM. In another embodiment, the effective amount of the MPO inhibitor is about 20 μM. In another embodiment, the effective amount of the MPO inhibitor is about 50 μM. In another embodiment, the effective amount of the MPO inhibitor is about 100 μM. In another embodiment, the effective amount of the MPO inhibitor is about 200 μM. In another embodiment, the effective amount of the MPO inhibitor is about 300 μM. In another embodiment, the effective amount of the MPO inhibitor is about 400 μM. In another embodiment, the effective amount of the MPO inhibitor is about 500 μM. In another embodiment, the effective amount of the MPO inhibitor is about 600 μM. In another embodiment, the effective amount of the MPO inhibitor is about 700 μM. In another embodiment, the effective amount of the MPO inhibitor is about 800 μM. In another embodiment, the effective amount of the MPO inhibitor is about 900 μM. In another embodiment, the effective amount of the MPO inhibitor is about 1 mM. In another embodiment, the effective amount of the MPO inhibitor is about 1.25 mM. In another embodiment, the effective amount of the MPO inhibitor is about 1.5 mM. In another embodiment, the effective amount of the MPO inhibitor is about 1.75 mM. In another embodiment, the effective amount of the MPO inhibitor is about 2 mM.

In one embodiment, MPO is downregulated by reducing the level of MPO mRNA in the subject. In certain embodiments, inhibiting or reducing MPO in the subject can be accomplished by reducing the amount of mRNA, e.g., via RNAi, or protein in the subject. MPO mRNA levels may be reduced, in one embodiment, using siRNA. SiRNA can be generated against MPO using sequences known in the art. For example, the following MPO sequences can be found in GenBank: NP_000241.1, NM_000250.1 [P05164-1]. Each of these sequences is incorporated herein by reference.

In certain embodiments, inhibiting or reducing MPO in the subject can be accomplished by reducing the amount of MPO protein in the subject via administration of an antibody that neutralizes or blocks the action of MPO (e.g., an anti-MPO antibody). An anti-MPO antibody can be an antibody that binds to, blocks, competes or interferes with binding or activity of MPO.

In certain aspects, it is beneficial to determine whether a subject who has previously had cancer may be at risk for a recurrence of the cancer caused by a reversal of dormancy of latent cancer cells remaining in the body. In certain embodiments, the cancer treated includes, but is not limited to, a solid tumor, a hematological cancer (e.g., leukemia, lymphoma, myeloma, e.g., multiple myeloma), and a metastatic lesion. In one embodiment, the cancer is a solid tumor. Examples of solid tumors include malignancies, e.g., sarcomas and carcinomas, e.g., adenocarcinomas of the various organ systems, such as those affecting the lung, breast, ovarian, lymphoid, gastrointestinal (e.g., colon), anal, genitals and genitourinary tract (e.g., renal, urothelial, bladder cells, prostate), pharynx, CNS (e.g., brain, neural or glial cells), head and neck, skin (e.g., melanoma or Merkel cell carcinoma), and pancreas, as well as adenocarcinomas which include malignancies such as colon cancers, rectal cancer, renal-cell carcinoma, liver cancer, non-small cell lung cancer, cancer of the small intestine, cancer of the esophagus. The cancer may be at an early, intermediate, late stage or metastatic cancer.

In one embodiment, the cancer is chosen from a lung cancer (e.g., a non-small cell lung cancer (NSCLC) (e.g., a NSCLC with squamous and/or non-squamous histology, or a NSCLC adenocarcinoma)), a skin cancer (e.g., a Merkel cell carcinoma or a melanoma (e.g., an advanced melanoma)), a kidney cancer (e.g., a renal cancer (e.g., a renal cell carcinoma (RCC) such as a metastatic RCC or clear cell renal cell carcinoma (CCRCC)), a liver cancer, a myeloma (e.g., a multiple myeloma), a prostate cancer (including advanced prostate cancer), a breast cancer (e.g., a breast cancer that does not express one, two or all of estrogen receptor, progesterone receptor, or Her2/neu, e.g., a triple negative breast cancer), a colorectal cancer, a pancreatic cancer, a head and neck cancer (e.g., head and neck squamous cell carcinoma (HNSCC), a brain cancer (e.g., a glioblastoma), an endometrial cancer, an anal cancer, a gastro-esophageal cancer, a thyroid cancer (e.g., anaplastic thyroid carcinoma), a cervical cancer, a neuroendocrine tumor (NET) (e.g., an atypical pulmonary carcinoid tumor), a lymphoproliferative disease (e.g., a post-transplant lymphoproliferative disease) or a hematological cancer, T-cell lymphoma, B-cell lymphoma, a non-Hodgkin lymphoma, or a leukemia (e.g., a myeloid leukemia or a lymphoid leukemia). In yet another embodiment, the cancer is a hepatocarcinoma, e.g., an advanced hepatocarcinoma, with or without a viral infection, e.g., a chronic viral hepatitis. In a certain embodiment, the subject has been treated previously for cancer.

In certain embodiments, the subject's cancer has been dormant for a period of time. The cancer may have been dormant for a period of months or years. In one embodiment, the cancer has been dormant for 6 months or more. In another embodiment, the cancer has been dormant for at least 1 year, 2 years, 3 years, 4 years, 5 years, or more.

In one embodiment, the subject is experiencing stress which has an impact on the beta-adrenergic signaling pathway. Such stress may be indicated by the presence of polymorphonuclear myeloid-derived suppressor cells (PMN-MDSC). PMN-MDSC represent the major population of MDSC (about 60-80%) and are characterized as CD11b⁺ CD14⁻ CD15⁺ and CD33⁺. PMN-MDSC may be identified, by methods known in the art. For example, as described in WO 2016/196451, PMN-MDSCs may be detected or monitored by contacting a population of PMN cells with a ligand that binds LOX-1 on the surface of the cell.

Thus, in another aspect, the presence of PMN-MDSC in a subject previously treated for cancer is detected. Once PMN-MDSC are detected, the subject is treated for cancer. In one embodiment, the treatment includes an inhibitor of S100A8/A9. In another embodiment, the treatment includes an inhibitor of FGFR. In yet another embodiment, the treatment includes an inhibitor of MPO.

In some embodiments, the level of S100A8/A9 is detected in a sample obtained from a subject. This level may be compared to the level of a control. In one embodiment, an increase in the level of S100A8, S100A9, or S100A8/A9 as compared to a control indicates a greater risk of reactivation of, or presence of reactivated, dormant tumor cells in the subject. In one embodiment, a level of 2500 ng/mL or higher is indicative of an increased risk of reactivation of dormant tumor cells in the subject, as compared to a control.

In one embodiment, the subject is then treated for cancer. In one embodiment, the treatment includes an inhibitor of S100A8/A9. In another embodiment, the treatment includes an inhibitor of FGFR. In yet another embodiment, the treatment includes an inhibitor of MPO.

In yet another embodiment, the methods described herein include treatment in combination with another cancer treatment or therapeutic agent to reduce or inhibit reversal of cancer cell dormancy, including known chemotherapeutic agents. The reduction or inhibition of cancer cell dormancy can be measured relative to the incidence observed in the absence of the treatment. The tumor inhibition can be quantified using any convenient method of measurement. Tumor inhibition can be reduced by about 10%, 20%, 30%, 40%, 50%, 60%, 70%, 80%, 90%, 95% or greater.

Chemotherapeutic agents (e.g., anti-cancer agents) are well known in the art and include, but are not limited to, anthracenediones (anthraquinones) such as anthracyclines (e.g., daunorubicin (daunomycin; rubidomycin), doxorubicin, epirubicin, idarubicin, and valrubicin), mitoxantrone, and pixantrone; platinum-based agents (e.g., cisplatin, carboplatin, oxaliplatin, satraplatin, picoplatin, nedaplatin, triplatin, and lipoplatin); tamoxifen and metabolites thereof such as 4-hydroxytamoxifen (afimoxifene) and N-desmethyl-4-hydroxytamoxifen (endoxifen); taxanes such as paclitaxel (taxol) and docetaxel; alkylating agents (e.g., nitrogen mustards such as mechlorethamine (HN2), cyclophosphamide, ifosfamide, melphalan (L-sarcolysin), and chlorambucil); ethylenimines and methylmelamines (e.g., hexamethylmelamine, thiotepa, alkyl sulphonates such as busulfan, nitrosoureas such as carmustine (BCNU), lomustine (CCNLJ), semustine (methyl-CCN-U), and streptozoein (streptozotocin), and triazenes such as decarbazine (DTIC; dimethyltriazenoimidazolecarboxamide)); antimetabolites (e.g., folic acid analogues such as methotrexate (amethopterin), pyrimidine analogues such as fluorouracil (5-fluorouracil; 5-FU), floxuridine (fluorodeoxyuridine; FUdR), and cytarabine (cytosine arabinoside), and purine analogues and related inhibitors such as mercaptopurine (6-mercaptopurine; 6-MP), thioguanine (6-thioguanine; 6-TG), and pentostatin (2′-deoxycofonnycin)); natural products (e.g., vinca alkaloids such as vinblastine (VLB) and vincristine, epipodophyllotoxins such as etoposide and teniposide, and antibiotics such as dactinomycin (actinomycin D), bleomycin, plicamycin (mithramycin), and mitomycin (mitomycin Q); enzymes such as L-asparaginase; biological response modifiers such as interferon alpha); substituted ureas such as hydroxyurea; methyl hydrazine derivatives such as procarbazine (N-methylhydrazine; MIH); adrenocortical suppressants such as mitotane (o,p′-DDD) and aminoglutethimide; analogs thereof derivatives thereof and combinations thereof.

In another embodiment, a method of inhibiting the recurrence of cancer in subject, comprising inhibiting stress-induced β-adrenergic pathway signaling is provided. In one embodiment, the method includes inhibiting or reducing S100A8/A9 in the subject.

Unless defined otherwise in this specification, technical and scientific terms used herein have the same meaning as commonly understood by one of ordinary skill in the art and by reference to published texts, which provide one skilled in the art with a general guide to many of the terms used in the present application.

The following examples are illustrative only and are not intended to limit the present invention.

### EXAMPLES

The mechanisms that contribute to reactivation of dormant tumor cells and cancer recurrence remain mostly unclear. Inflammation is implicated in supporting growth of disseminated tumor cells (Gay and Malanchi, 2017). However, epidemiological evidence directly linking inflammation and infections with cancer recurrence is lacking. Myeloid cells are a critical component of any inflammatory process and major part of tumor microenvironment (TME). They include populations of macrophages (M(D), dendritic cells (DC), neutrophils (PMN), and monocytes (MON). Accumulation of pathologically activated immune suppressive PMN and MON termed polymorphonuclear myeloid-derived suppressor cells (PMN-MDSC) and monocytic MDSC (M-MDSC) is one of the prominent features of cancer and chronic inflammation (Veglia et al., 2018). These cells contribute to tumor progression via multiple mechanisms and their accumulation has been shown to correlate with cancer stage and poor response responses to therapy (Gabrilovich, 2017; Martens et al., 2016; Sacdalan et al., 2018; Wang et al., 2018). Thus, MDSC may represent a starting point in understanding the possible contribution of pathologically activated cells for reactivation of dormant cells.

Here, we identified the mechanism of reactivation of dormant tumor cells by PMN linking stress and inflammation. Epidemiological and clinical studies have provided strong evidence for links between chronic stress and cancer incidence as well as cancer progression (Chiriac et al., 2018; Moreno-Smith et al., 2010). Cognitive-behavioral stress management delivered after surgery reduced risk of tumor recurrence and mortality in patients with non-metastatic breast cancer (Stagl et al., 2015). Patients with surgically treated breast cancer who had developed recurrence later displayed higher neutrophil, lymphocyte, and natural killer cell counts, as well as higher cortisol level than patients who did not recur (Thornton et al., 2008). We found that stress hormones via 02-adrenergic receptors induced massive release of pro-inflammatory S100A8/A9 complexes by PMN without affecting their viability. These proteins, in autocrine and paracrine fashion, caused accumulation of oxidized lipids in PMN, which upon release directly activated proliferation of dormant tumor cells via up-regulation of fibroblast growth factor receptor pathway.

### Example 1: Materials and Methods

### Cancer Patients

Serum samples were collected from 80 individuals (10 males and 50 females) aged 50-84 diagnosed with stage I-II NSCLC who underwent tumor resection at New York University Medical Center. Informed consent was obtained from all patients and study was approved by NYU Medical Center Institutional Review Board (Protocol H8896, H.P. Col). Tumor recurrence was evaluated during regular follow up visits. We evaluated samples collected 3 months after surgery. This time frame was selected to avoid direct effect stress associated with operation and post-operative manipulations. No patients had a recurrence within 3-month period after surgery.

### Mice

NOD/SCID and C57BL/6 mice were obtained from Taconic. S100A9KO were described earlier (Manitz et al., 2003). MPO KO mice (B6.129X1-Mpotm1Lus/J) were obtained from Jackson Lab. Mice were housed in pathogen free animal facility at Wistar Institute and all experiments were approved by Wistar Institute IACUC. Male and female mice were equally represented in each experiment. Mice were 6-8 week old.

### Establishment of Dormant Cells

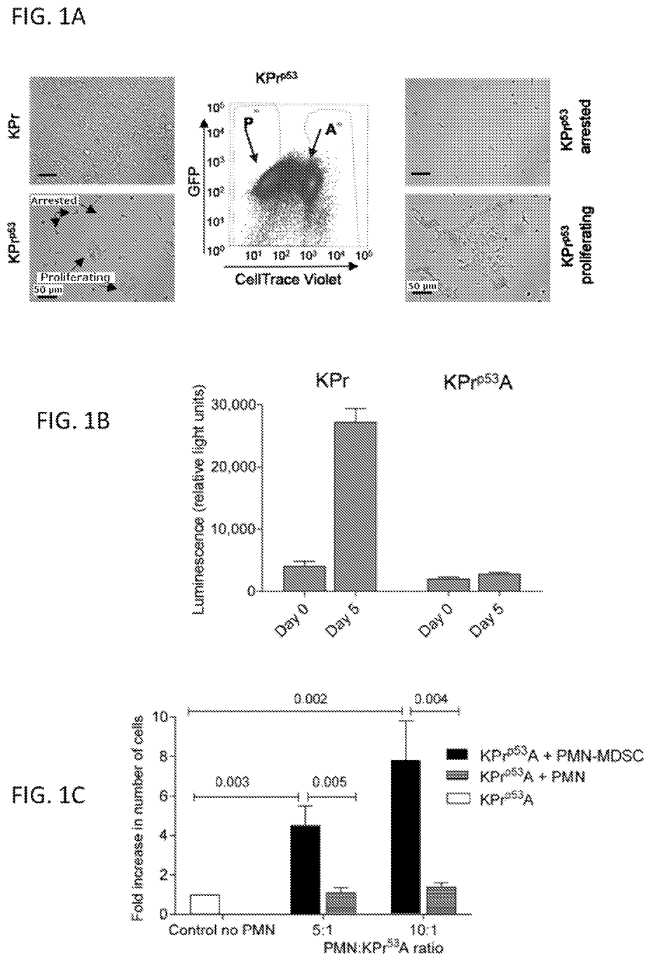

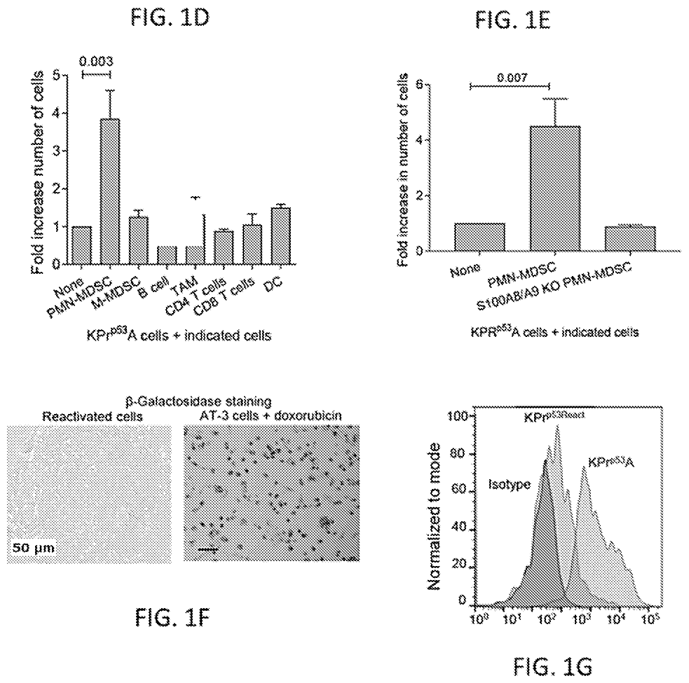

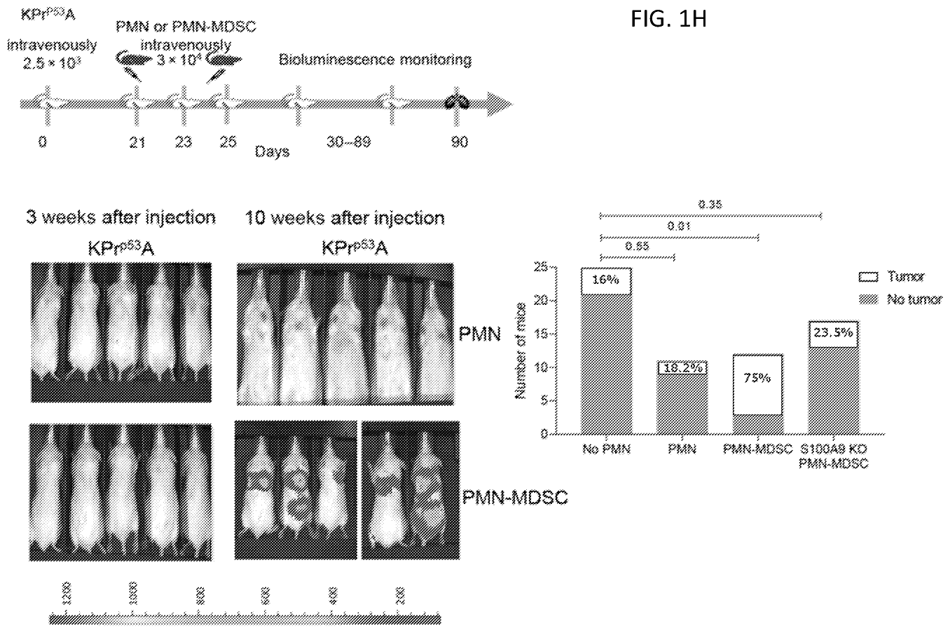

The KPr cell line (16) was maintained in RPMI 1640 medium (Corning) and 10% FBS (Gibco). Treatment of KPr cells with 250 nM 4-hydroxytamoxifen (4-OHT, Sigma-Aldrich) induced p53 expression. p53-induced cells (KPr^{p53}) were stained with CellTrace dye for 20 min at 37° C. according to the manufacturer's instructions (Life Technologies) and seeded at 8500 cells/cm² in culture in a T175 tissue culture-treated flask (Gibco). After 7 days, cells were detached and sorted based on the CellTrace dye intensity as proliferating (KPr^{p53}P, with lower CellTrace) and arrested cells (KPr^{p53}A, with higher-intensity CellTrace; see FIG. 1A for representative image). LL2 cells were seeded at 1000 cells/cm2 and treated for 3 days with 2.5 μM cisplatin (Selleckchem). After that, cells were stained with CellTrace and sorted as described above. OVCAR3 cell senescence was induced by treating 5×104 cells/cm2 with 0.5 μM cisplatin for 72 hours. After that time, cells were stained with CellTrace and sorted. A549 senescence was induced by 72-hour treatment with 4 μM cisplatin and cells were sorted after staining with CellTrace. Time points for sorting were chosen as the best to observe separation between arrested and proliferating cells. Cells were sorted on Astrios (Beckman Coulter) or Melody (Becton Dickinson) fluorescence-activated cell sorters.

### Cell Proliferation

Two thousand cells per well were seeded in 96-well clear bottom plate in triplicate (Lonza). Sixteen hours after seeding, NE (Sigma-Aldrich) at 0.5 to 5 μM final concentration or BGJ398 at 0.75 μM final concentration (Selleckchem) was added to culture. To test the effect of BGJ398t on KPr^{p53}A^{React} cells, escalating doses ranging from 0 to 10 μM final concentrations were used. To detect luciferase activity (proportional to number of cells in culture), Luciferin (PerkinElmer) was added 1:200 from stock according to the manufacturer's instructions and luminescence was measured with a Victor spectrophotometer (PerkinElmer).

### Cell Isolation

PMN or PMN-MDSC were isolated from spleen of tumor-free mice or LLC tumor-bearing mice, respectively. A total of 5×10⁵ LLC cells were injected into the right flank of mice, and spleens were harvested when tumors reached 200 mm². Spleens were mechanically dissociated. Red blood cells were lysed with ACK buffer cells. Splenocytes were stained with mouse biotin-conjugated Ly6G antibody (Miltenyi Biotec) followed by biotin microbeads. Ly6G+ cells were purified by magnetic separation with MACS column for magnetic cell isolation, according to the manufacturer's instructions (Miltenyi Biotec).

B and T cells were isolated with magnetic beads using CD19, CD4, or CD8 antibodies. T cells were maintained for 48 hours with IL-2 (100 U/ml) before experiments. Macrophages were sorted with a MoFlo AStrios cell sorter (Beckman Coulter) from spleens of tumor-bearing mice as CD11b⁺Ly6C⁻F4/80⁺ cells after exclusion of dead cells and doublets. Mouse macrophages/DCs were also generated from enriched BM hematopoietic progenitor cells (HPCs) after HPC isolation using a lineage depletion kit per the manufacturer's instructions (Miltenyi Biotec). Cells were seeded at 50,000 cells/ml in 24-well plates and granulocyte-macrophage colony-stimulating factor (GM-CSF; 10 ng/ml) was added to the culture at days 0 and 3. On day 6, cells were collected and CD11*c cells were isolated by staining with anti-CD11c-conjugated microbeads and separated with MACS column for magnetic cell isolation, according to the manufacturer's instructions (Miltenyi Biotec).

In Vitro Co-Culture with PMN and PMN-MDSC

Arrested tumor cells were plated at 100 cells/cm² in a 48-well tissue culture-treated plate (Costar) for coculture experiments. After 24 hours, PMN-MDSC or PMN were added at indicated ratios. Cocultures were maintained for 48 hours in RPMI 1640+10% FBS in the presence of mouse recombinant GM-CSF (10 ng/ml; Rocky Hill) to ensure neutrophil viability. All control conditions were in the presence of media with GM-CSF. All indicated supplements (NE, S100A8/A9, BGJ398, ICI 118-551, IL-2, MPO inhibitor, LPS, lipids, PMA, and thapsigargin 1 μM) were added to culture at the same time as PMN and maintained for 48 hours. After that, media were removed and replaced by fresh media without GM-CSF. After 1 week, GFP⁺ cells in wells were counted via an inverted microscope. When lipid extracts were used, lipids were added to dormant cells in culture at 1 to 0.05 or 0.01 nmol as indicated in each experiment.

In Vivo Experiments with Dormant Cells

A total of 2.5×10⁴ KPr^{p53}A cells were intravenously injected to NOD/SCID mice. Beginning 3 weeks after injection, mice were imaged weekly. Luciferin (PerkinElmer) was intraperitoneally injected in mice before imaging according to the manufacturer's instruction. Mice were imaged with IVIS Spectrum In Vivo Imaging System (IVIS, PerkinElmer) to detect luminescent signal coming from growing cancer cells. Mice were put under anesthesia in the induction chamber with isoflurane vaporizer set to 2.5%, at a flow rate of ˜1.5 liters/min. Mice were then transferred in the IVIS imaging stage nose cone inside the IVIS manifold (IVIS manifold nose cones flow set to ˜0.25 LPM). Animals were monitored by video during imaging. Once the images of luciferase/luciferin activity were acquired, the anesthesia was turned off and the animals were returned to their cages. Only animals lacking luminescent signal (about 90% of the mice) were enrolled in further studies. PMN or PMN-MDSC were intravenously injected at 0.3×106 cells per mouse three times every other day. Analysis and quantification were performed with IVIS imaging system Living Image Software 4.7.4 (PerkinElmer). Photon counts in the area of interest were reported after subtraction of background. When signal was detected, mice were sacrificed, and bioluminescent images of the harvested lung were taken. Mouse lungs and livers were embedded in optimal cutting temperature (OCT) compound (Thermo Fisher Scientific) after exposure to increased concentration of sucrose (5, 15, 20, and 30%) and frozen for immunofluorescence (IF) analysis. If no bioluminescence was detected in lungs after 80 days from last PMN injection, experiments were terminated, and lung tissue were collected. LL2^{Cis}A cells were intravenously injected at 0.5×10⁵ into C57BL/6 mice. Bioluminescence was not detected in any LL2^{Cis}A-injected mice at 3 weeks after injection. Mice were treated with PMN or PMN-MDSC and analyzed as described above.

### In Vivo Stress Induction

To induce stress, individual mice were restrained in individual semi-cylindric plastic restrainer (BrainTree scientific, Braintree, Mass., USA) 4h/day 5 days a week starting 1 week before PMN injection for a total of 3 weeks. Mice were allowed to move back and forward the tube, but they could not completely turn. When study included β-blocker treatment, 1 mg/kg ICI-118, 5551 (Selleckchem) was injected i.p. daily to the mice, during the stress induction period. In studied with Tasquinimod, mice injected with dormant cells were treated with Tasquinimod diluted in drinking water at final concentration of 0.15 mg/ml during the stress induction period. Water was replenished twice a week and kept in dark bottle, to exposure to light.

### IF and Immunohistochemistry

For GFP detection, frozen tissues were cut 5 μm thick on a CM1950 cryostat (Leica), placed on superfrost plus microscope slides (Fisher Scientific), and fixed with 4% paraformaldehyde (Sigma-Aldrich) for 10 min at room temperature (RT). Tissues were stained for nuclei detection with Hoechst 33342 or 4′,6-diamidino-2-phenylindole (Thermo Fisher Scientific) diluted at 5 μg/ml for 10 min at RT. For Ly6G staining, tissues were fixed with 4% paraformaldehyde in phosphate-buffered saline (PBS) for 10 min at RT. After washing, blocking was achieved with 1-hour incubation at RT with PBS/bovine serum albumin (BSA) 5%. Slides were stained with Alexa Fluor 594-conjugated rat anti-mouse Ly6G (BioLegend, Clone 1A8) at 1:100 and incubated at 4° C. overnight in a humid chamber. After washing, nuclei were stained as described above. Coverslips were added with ProLong Diamond Antifade Mountant (Thermo Fisher Scientific), and images were acquired with a Nikon 80i upright microscope (Nikon).

For p53 or H3K9Me staining, cells were fixed in a 24-well plate with 4% paraformaldehyde in PBS for 10 min at RT and then washed with PBS and 1% BSA. Cells were permeabilized with 0.1% Triton X-100 solution in PBS for 20 min at RT. After washing, samples were blocked with 5% PBS/BSA for 1 hour at RT. Rabbit anti-mouse p53 (Leica) antibody or rabbit-anti-mouse Tri-Methyl-Histone H3 (Lys9) antibody (Cell Signaling) was added at a 1:200 dilution in PBS/1% BSA and incubated for 3 hours at 37° C. After washing, slides were then incubated with anti-rabbit Alexa 549 (Life Technologies) secondary antibody at a 1:500 dilution for 2 hours at RT. Nuclei were stained as described above. Images were acquired at the TIE2000 inverted microscope (Nikon). All images were then processed with ImageJ software. For hematoxylin and eosin staining, mice were euthanized and lungs were harvested. The lungs were formalin-fixed and then paraffin-embedded. Tissue sections of 5 μm thick were stained with hematoxylin and eosin, and images were acquired with a Nikon 80i upright microscope (Nikon).

### Flow Cytometry

KPr cells and KPr^{p53React} cells were plated on day 0 in complete RPMI 1640 medium supplemented with 10% FBS. After 24 hours, cells were pulsed with BrdU according to the manufacturer's instructions (Becton Dickinson). BrdU was removed and replaced with fresh medium. On day 2, cells were exposed to tamoxifen to restore p53 (KPr^{p53}). On day 4, cells were stained with CellTrace Violet as described above while attached to the plate to minimize handling. On day 10, cells were harvested and stained with an anti-BrdU PE-conjugated antibody according to the manufacturer's BrdU Flow Kit protocol (BD Biosciences). For cell cycle analysis, cells were fixed in 70% ethanol and stained with FxCycle PI/ribonuclease (RNase) staining solution (BD Biosciences) according to the manufacturer's instruction. Samples were analyzed using a BD LSRII cytometer (Becton Dickinson) and data were analyzed by FlowJo 10.5 software (Becton Dickinson).

### Norepinephrine and S100A8/A9 Measurements

One hundred microliters of mouse peripheral blood was collected from facial vein. NE was measured by a mouse NE ELISA Kit (Novus Biologicals). S100A8/A9 was measured by Mouse S100A8/S100A9 Heterodimer DuoSet ELISA (R&D Systems). S100A8/A9 concentrations in human serum samples were determined by using a double-sandwich ELISA system as previously described (64), which is different from commercial ELISA kits. Human NE was measured using an ELISA kit (Abnova).

### MPO Activity in Neutrophils

Neutrophils were isolated from tumor-free mice as described above, and MPO activity was measured with an MPO Fluorometric Activity Assay Kit (Sigma-Aldrich) using a VICTOR spectrophotometer (PerkinElmer). Absorbance was measured every 5 min until plateau was reached, and MPO activity was calculated with the formula suggested by the manufacturer.

### β-Galactosidase Activity

Arrested or proliferating cells from all models used in the study (KPr, LL2, A549, and OVCAR3) were plated at 5×10⁴ cells/ml and incubated overnight. Cells were then stained for β-galactosidase using senescence β-galactosidase Staining Kit (Cell Signaling) per the manufacturer's instructions.

### Western Blot

Cells were lysed in radioimmunoprecipitation lysis buffer supplemented with phosphatase inhibitors (Roche). Thirty to forty micrograms of protein were loaded in each lane of 4 to 12% Nupage gradient gels (Thermo Fisher Scientific). Gels were run on a Nupage apparatus in Nupage 1× running buffer and transferred in a wet system with running buffer (Thermo Fisher Scientific). Membranes were blocked for 2 hours at RT in Odyssey blocking buffer (LI-COR Biosciences) and then primary antibodies 1:500 in PBS+1% Tween (Thermo Fisher Scientific) were added. IRDye 800CW Goat anti-Mouse immunoglobulin G (LI-COR Biosciences) secondary antibodies were incubated for 2 hours at RT in PBS+1% Tween at 1:10,000 dilution. Membranes were imaged using an Odyssey imaging system (LI-COR Biosciences).

### NET Detection

PMN were cultured for 8 hours in 24-well flat-bottom plates in RPMI 1640+10% FBS. PMN were then fixed with 4% paraformaldehyde (Electron Microscopy Sciences) followed by staining with SYTOX Green Nucleic Acid Stain (SYTOX) at a final concentration of 250 nM. A Nikon TE300 inverted microscope equipped with a motorized XY stage was used to image NETs by acquiring 25 random locations per well. The z-stacks per location were then combined into an extended depth focused image. The total NET area was calculated by segmenting each image using a defined threshold pixel intensity setting. The spot detection tool in NIS-Elements Advanced Research (Nikon) was used to count the number of cells per field. The sum of the total NET area in the 25 random fields of view was divided by the total number of cells in the 25 fields of view to obtain NET area (in micromolar) per cell.

qRT-PCR

RNA from tumor cells or PMN was extracted from snap-frozen pellets with Quick-RNA Microprep or Quick-RNA Miniprep (Zymo Research) per the manufacturer's instructions. cDNA was prepared with a High-Capacity cDNA Reverse Transcription Kit with RNase Inhibitor (Thermo Fisher Scientific) per the manufacturer's instructions. qRT-PCR was performed in an ABI QuantStudio 5 Rm 422 machine (Applied Biosystems).

### RNA Sequencing

Total RNA was extracted from cell pellets using the Direct-zol RNA Miniprep (Zymo Research) per the manufacturer's instructions. RNA quality was validated using the TapeStation RNA ScreenTape (Agilent). One hundred nanograms of total RNA was used to prepare a library for Illumina Sequencing using the Quant-Seq 3′mRNA-Seq Library Preparation Kit (Lexogen). Library quantity was determined using a qPCR kit and absolute quantification with standard curve method (KAPA Biosystems). Overall library size was determined using the Agilent TapeStation and the DNA High Sensitivity D5000 ScreenTape (Agilent). Equimolar amounts of each sample library were pooled and denatured, and Mid-Output, Paired-End, 150-cycle Next-Generation Sequencing was done on a NextSeq 500 (Illumina). Data were aligned using bowtie2 (65) against mm10 genome, and gene-level read counts were estimated with RSEM v1.2.12 software (66) for ensemble transcriptome. DESeq2 (67) was used to estimate significance of differences between any two experimental groups, and genes that changed at least twofold with an FDR threshold less than 5% were considered significantly different. Gene set enrichment analysis was done using QIAGEN's Ingenuity Pathway Analysis software (IPA, QIAGEN) using the “Canonical Pathways” option. Activation states of pathways were predicted and Z scores were calculated by IPA based on known information about roles of membership genes and their direction of change. Pathways with significantly predicted activation scores (|Z score|>2) were reported. Known upstream regulators and protein-protein interaction partners for Fgfr1 and Fgfr2 were derived from Ingenuity Knowledgebase. Gene expression data were deposited to Gene Expression Omnibus accession GSE153944.

### LC-ESI-MS Analysis of Lipids

Lipids were extracted by the Folch procedure with slight modifications, under nitrogen atmosphere, at all steps. Briefly, methanol (1 ml) was added to the cell suspension and mixed. After that, chloroform (2 ml) was added, and the mixture was vortexed every 15 min for 1 hour at 0° C. Next, 0.1 M NaCl (0.5 ml) was added to the samples and vortexed, and the chloroform layer was separated by centrifugation (1500 g, 5 min). The lower (organic) layer was collected. The aqueous layer was re-extracted with 1 ml of chloroform/methanol (2:1, v/v). The chloroform (lower) layers were combined, evaporated under a stream of nitrogen, and used for lipidomics analysis. MS analysis of phospholipids was performed on a Fusion Lumos trihybrid-quadrupoleorbitrap-ion trap mass spectrometer (Thermo Fisher Scientific). Phospholipids were separated on a normal phase column [Luna 3 μm Silica (2) 100 A, 150×1.0 mm (Phenomenex)] at a flow rate of 0.065 ml/min on a Thermo Ultimate 3000 HPLC system. The column was maintained at 35° C. The analysis was performed using gradient solvents (A and B) containing 10 mM ammonium formate. Solvent A contained propanol/hexane/water (285:215:5, v/v/v) and solvent B contained propanol/hexane/water (285:215:40, v/v/v). All solvents were LC/MS grade. The column was eluted for 0 to 3 min with a linear gradient from 10 to 37% B and then held for 3 to 15 min at 37% B. The column was eluted for 15 to 23 min with a linear gradient of 37 to 100% B, and then held for 23 to 75 min at 100% B. The column was eluted again for 75 to 76 min with a linear gradient from 100 to 10% B followed by equilibration from 76 to 90 min at 10% B. Analysis was performed in negative ion mode at a resolution of 120,000 for the full MS scan in a data-dependent mode. The scan range for MS analysis was 400 to 1800 m/z (mass/charge ratio) with a maximum injection time of 100 ms using 1 microscan. An isolation window of 1.2 Da was set for the MS and MS2 scans. Capillary spray voltage was set at 3.5 kV, and capillary temperature was 320° C.

### MPO Lipid Treatment

Lipids extracted from PMN were dried under N2 and then were re-suspended in 200 μl of 20 mM PBS (pH 7.4) containing 100 mM NaCl and 100 μM diethylenetriaminepentaacetic acid (DTPA) and incubated with 28 nM MPO (Sigma-Aldrich) and 50 μM H₂O₂ for 1 hour at 37° C. H₂O₂ (2 μl of 5 mM) was added every 5 min. Individual molecular species of phospholipids, including PE (18:0p/20:4), PE (18:0/20:4), PC (18:0p/20:4), PC (18:0/20:4), and PC (18:0p/22:6) (Avanti Polar Lipids), were treated separately by 56 nM MPO in 20 mM PBS at pH 7.4 in the presence of 100 μM NaCl and 100 μM DTPA for 1 hour at 37° C. H₂O₂ (50 μM) was added every 5 min. In addition, individual phospholipids were incubated with 250 μM NaClO in 20 mM PBS at pH 7.4 in the presence of 100 mM NaCl and 100 μM DTPA for 1 hour at 37° C.

### Statistical Analyses

After testing for normal distribution of data, statistical analyses were performed using two-tailed Student's t test and GraphPad Prism 5 software (GraphPad Software Inc.). All data are presented as means±SEM, and P values less than 0.05 were considered significant. Fisher's exact test and Boschloo's test were used for analysis of categorical data. One-way analysis of variance (ANOVA) test with correction for multiple comparisons (Kruskal-Wallis or Tukey's tests) was used in experiments with more than two groups. A nonparametric Spearman test was used to calculate correlation coefficients, and one-sided P values were calculated. One-sided test was selected because the hypothesis stated only one-directional changes in the data.

### Example 2: Reactivation of Dormant Tumor Cells

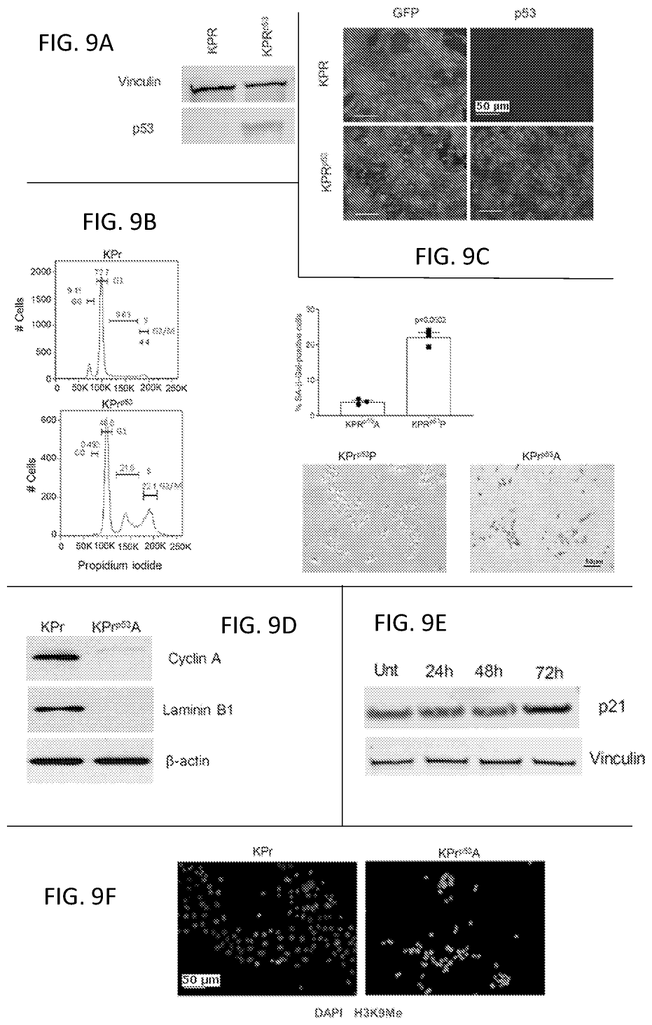

To investigate tumor dormancy, we generated a mouse model of disseminated dormant tumor cells. To accomplish this, mice expressing the KRAS^{G12D} allele, which are prone to lung adenocarcinoma, were crossed to a dual transgenic mouse expressing Trp53^{Lox-STOP-Lox} and Rosa26^{CreER} alleles. Spontaneously arising tumors were used to derive Kras^{G12D/+;}Trp53^{LSL/LSL};Rosa26^{Cre-ER/creER} (KPr) cell lines, which were further modified to express both luciferase and green fluorescent protein (GFP) for in vitro and in vivo monitoring. Tamoxifen-mediated activation of Cre^{ER} facilitates deletion of the transcriptional STOP cassette embedded in the first intron of the Trp53 locus, resultin in the expression of endogenous p53. This mediates cell cycle arrest and senescence (16, 17). After exposure to tamoxifen and restoration of p53 expression (KPr^{p53}) (FIG. 9A), a subset of KPr^{p53} cells were arrested in the G2-M phase of cell cycle (FIG. 9B). KPrp53-arrested (KPr^{p53}A) cells were sorted from proliferating cells (KPr^{p53}P) based on retention of CellTrace Violet proliferation dye (FIG. 1A). When seeded at low density (1000 cells/cm²), KPr^{p53}A cells did not proliferate for at least 10 days (FIG. 1A and FIG. 1B). Consistent with a p53-mediated senescence-like response, KPr^{p53}A cells were positive for β-galactosidase activity (FIG. 9C), lacked expression of cyclin A and laminin B1 (FIG. 9D), had modest but clearly detectable increases in p21 (FIG. 9E), and expressed lysine 9-trimethylated histone H3 (H3K9Me3) (FIG. 9F).

We next evaluated the ability of different myeloid cells isolated from tumor-bearing or tumor-free mice to reactivate proliferation of KPr^{p53}A cells. While addition of CD11b⁺Ly6C^{lo}Ly6G⁺ PMN from tumor-free mice at a 5:1 ratio had no effect on the number of KPr^{p53}A cells after 5 days in culture, addition of PMN-MDSC with the same phenotype isolated from Lewis lung carcinoma (LLC)-bearing mice resulted in proliferation of KPr^{p53}A cells (FIG. 1C). In contrast, none of other tested myeloid or lymphoid cells were able to reactivate proliferation of KPr^{p53}A cells even at high (10:1) effector/tumor cell ratios (FIG. 1D).

One of the most prominent factors that distinguish PMN-MDSC from PMN is high expression of S100A8/A9 proteins (18). S100A8 and S100A9 are low-molecular weight intracellular calcium-binding proteins (19). Deletion of S100A8/A9 markedly reduced the suppressive activity of MDSC (20, 21). S100A8/A9 has diverse intracellular functions. These proteins are involved in uptake and transport of arachidonic acid (22), NADPH (reduced form of nicotinamide adenine dinucleotide phosphate) oxidase activity, and reactive oxygen species (ROS) production (23). S100A9 could regulate PMN-MDSC suppressive function via increased expression of Ptges and PGE2 production (21).

We explored the involvement of S100A8/A9 proteins in the ability of PMN-MDSC to reactivate proliferation of KPr^{p53}A tumor cells by using S100A9 knockout (KO) mice (24). These mice also do not express S100A8 protein, thus making them functional double KO. PMN-MDSC isolated from LLC TB (tumor-bearing) S100A8/A9 KO mice were not able to activate proliferation of KPr^{p53}A cells in vitro (FIG. 9E). Last, to confirm that reactivated KPr^{p53}A cells (KPr^{p53}A^{React}) are proliferating, we show that the cells lose β-galactosidase activity (FIG. 1F) and demonstrate incorporation of the proliferation label, bromodeoxyuridine (BrdU) (FIG. 1G).

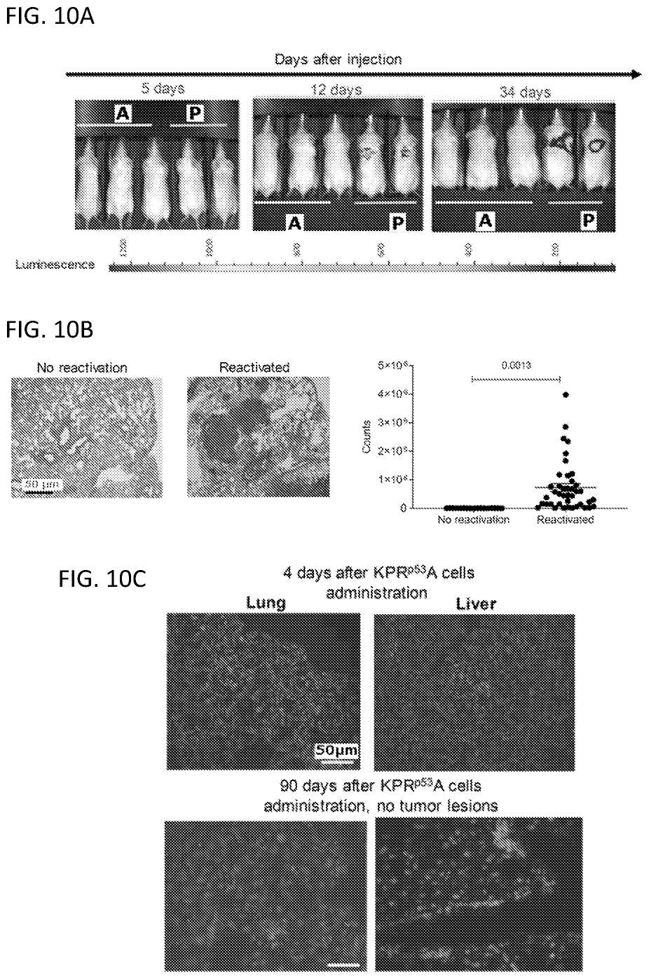

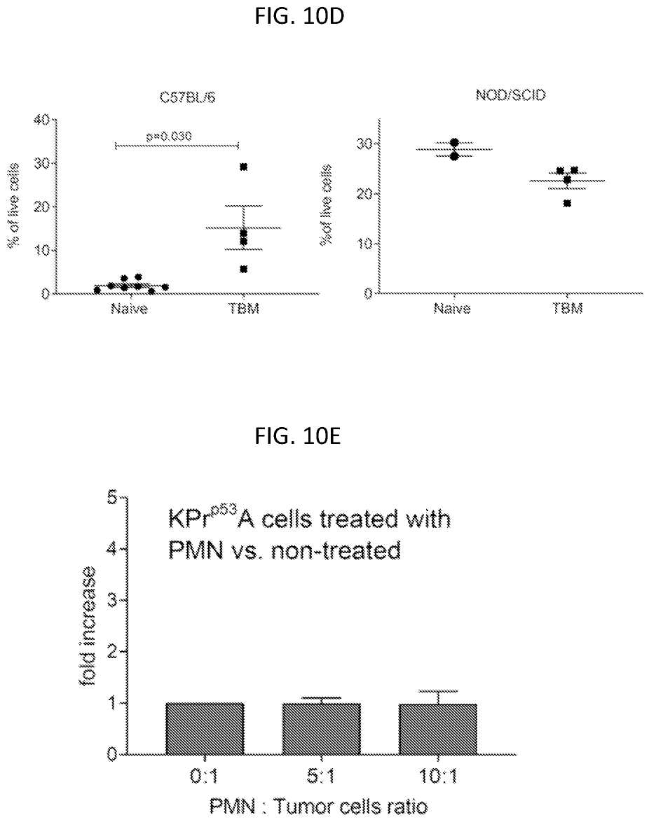

Immunodeficient nonobese diabetic (NOD)/severe combined immunodeficient (SCID) mice were used to assess tumor dormancy in vivo. After intravenous administration of KPr^{p53}P to these mice, lung tumor lesions became detectable via bioluminescence imaging within 2 weeks; conversely, administration of KPr^{p53}A cells did not form detectable lesions after 10 weeks (FIG. 1H and FIG. 10A). Tumor lesions were readily detectable by immunohistochemistry in lungs of mice injected with KPr^{p53}P cells (FIG. 10B). When lung tissues from mice injected with KPr^{p53}A cells were evaluated by microscopy, single KPr^{p53}A cells were detectable (FIG. 10C), indicating that single tumor cells were present in lung but did not proliferate.

In contrast to C57BL/6 wild-type (WT) mice, PMN were not expanded in NOD/SCID LLC TB mice (FIG. 10D) and PMN from these mice failed to reactivate proliferation of KPr^{p53}A cells (FIG. 10E). Thus, the NOD/SCID model allows for evaluation of the effect of exogenous PMN. KPr^{p53}A cells were intravenously injected into NOD/SCID mice. One week later, PMN from the spleen of naïve C57BL/6 mice, PMN-MDSC from the spleen of WT LLC-bearing mice, or PMN-MDSC from the spleen of S100A8/A9 KO LLC-bearing mice were intravenously transferred three times every other day. Ten weeks after the KPr^{p53}A cell transfer, 16% of control mice developed tumor lesions in lungs (FIG. 1H). Transfer of PMN did not affect that frequency. In contrast, 75% of mice injected with WT PMN-MDSC developed lung tumors. This effect was completely abrogated when PMN-MDSC were transferred from S100A8/A9 KO LLC TB mice (FIG. 1H). Thus, PMN-MDSC from tumor-bearing mice can reactivate dormant tumor cells, and reactivation depends on expression of S100A8/A9 proteins by PMN-MDSC.

### Stress-Induced S100A8/A9 Regulates Reactivation of Dormant Tumor Cells by Neutrophils

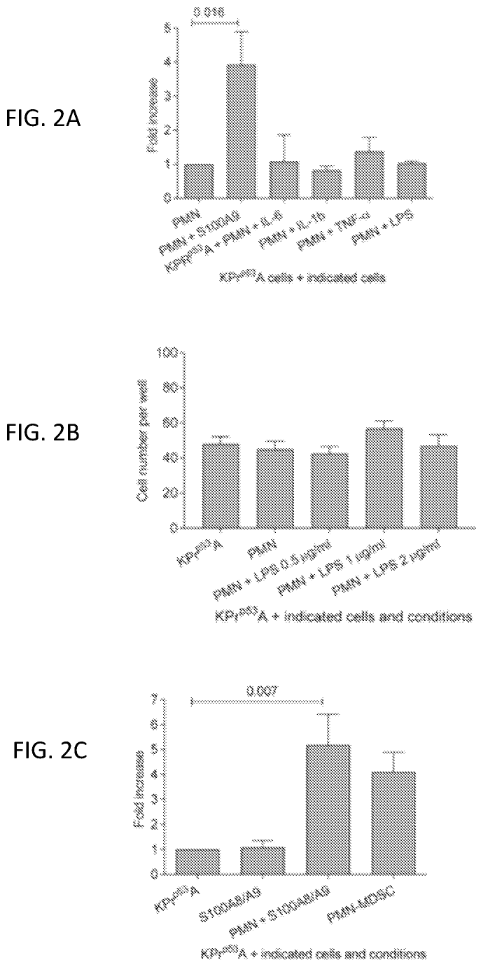

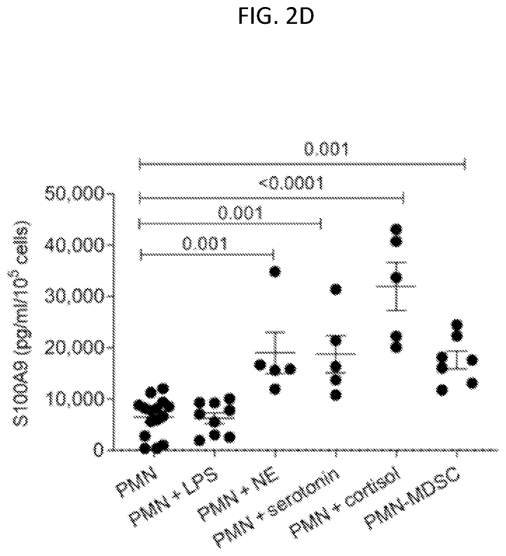

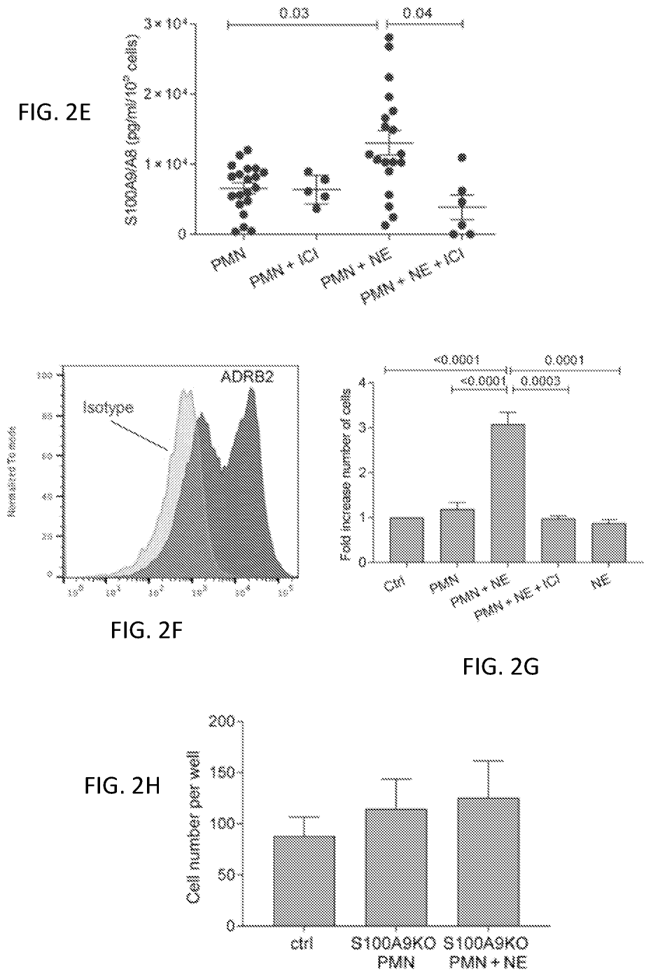

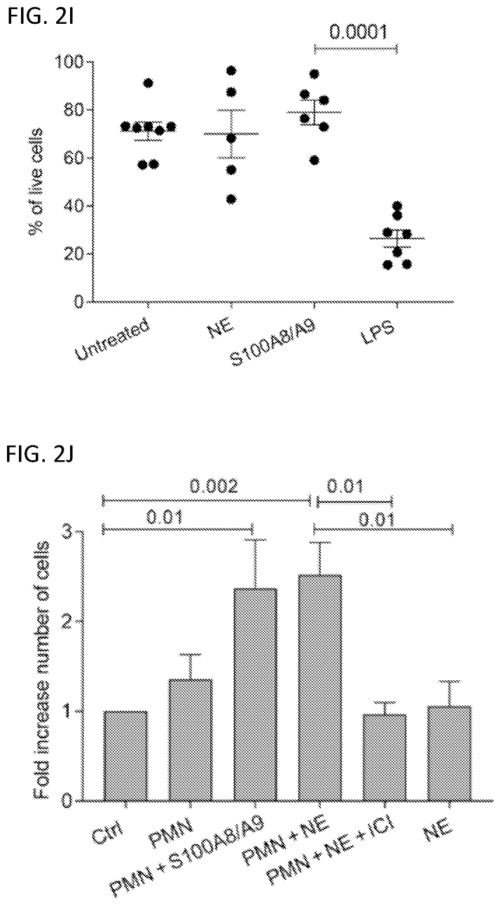

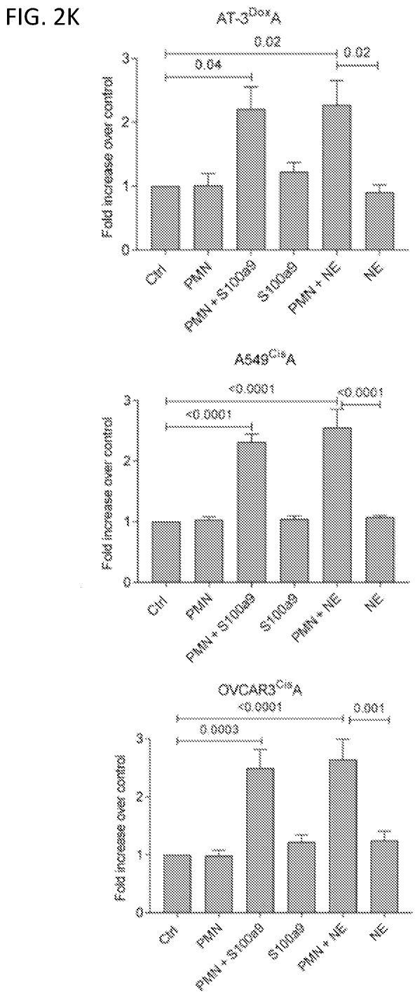

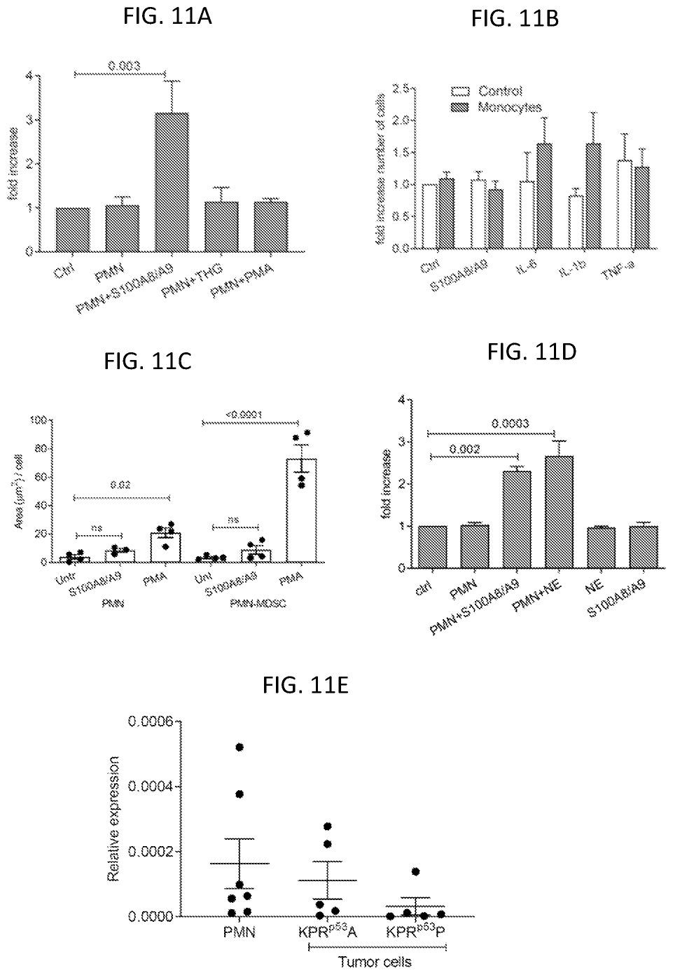

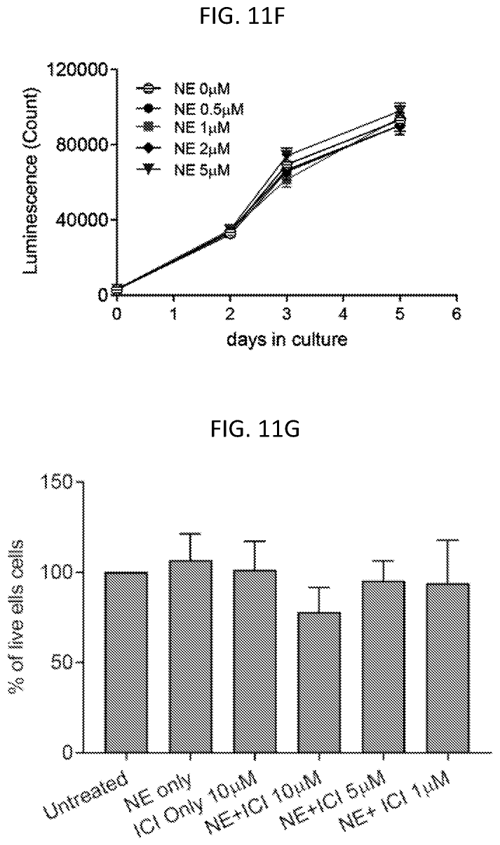

In the absence of tumor burden, mice and humans lack PMN-MDSC (14). We therefore investigated the conditions that could induce PMN to acquire the ability to reactivate dormant tumor cells in the absence of tumor burden by mimicking the events leading to cancer recurrence. Incubation of PMN with proinflammatory cytokines such as interleukin-10 (IL-10), tumor necrosis factor-α (TNF a), or IL-6 (FIG. 2A); phorbol 12-myristate 13-acetate (PMA); or the endoplasmic reticulum (ER) stress inducer thapsigargin (FIG. 11A) did not induce their ability to reactivate dormant KPr^{p53}A cells. Lipopolysaccharide (LPS) at concentrations ranging from 0.5 to 2 μg/ml also did not affect the ability of PMN to reactivate KPr^{p53}A cells (FIG. 2A and FIG. 2B). In contrast, addition of recombinant S100A8/A9 enabled PMN to induce proliferation of KPr^{p53}A cells. In the absence of PMN, there was no effect of S100A8/9 on reactivation of KPr^{p53}A cells (FIG. 2C). Likewise, incubation of S100A8/A9 with MON failed to reactivate KPr^{p53}A cells (FIG. 11B). Thus, exogenous S100A8/A9 treatment of PMN phenocopied the effect of PMN-MDSC on dormant cells.

Because neutrophil extracellular traps (NETs) were previously implicated in reactivation of dormant cells (25), we tested the effect of S100A8/A9 protein on NET formation by PMN and PMN-MDSC. S100A8/A9 did not cause substantial up-regulation of NETs (FIG. 11C). Citrullination of histones by peptidyl arginine deiminase 4 (PAD4) is central for NET formation, and PMN isolated from PAD4 KO mice are not able to form NETs (26). PMN isolated from PAD4 KO mice induced proliferation of KPr^{p53}A cells in the presence of S100A8/A9 (FIG. 11D), indicating that the effect of S100A8/A9 proteins on PMN is not mediated by NET formation.

We then investigated factors that could affect the release of S100A8/A9 protein by PMN. Epidemiological and clinical studies have provided strong evidence linking chronic stress and cancer progression (27, 28). Therefore, we tested the effect of stress hormones such as epinephrine, norepinephrine (NE), cortisol, and serotonin on S100A8/A9 release by PMN. Treatment of PMN with these hormones, but not with LPS, caused rapid release of S100A8/A9 proteins. The specific β2-adrenergic receptor antagonist (β-blocker) ICI-118,551 abrogated the NE-mediated secretion of S100A8/A9 by PMN (FIG. 2D and FIG. 2E). We then focused on NE, because it has previously been implicated in promotion of tumor proliferation (29) and metastasis in the lungs (30) and has shown a direct effect on myeloid cells (31). PMN express a β2-adrenergic receptor (FIG. 2F). Addition of NE to the culture of PMN with KPr^{p53}A caused proliferation of tumor cells, and this effect was abrogated by ICI-118,551 (FIG. 2G). PMN from S100A8/A9 KO mice failed to reactivate dormant tumor cells in the presence of NE (FIG. 2H), indicating that the effect of S100A8/A9 was downstream of NE. NE alone did not affect the proliferation of KPr^{p53}A cells, although tumor cells express 32 receptor (FIG. 11E and FIG. 11F). Overnight incubation of PMN with NE and S100A8/A9 did not affect PMN viability, whereas LPS markedly reduced it (FIG. 2I). In addition, ICI-118,551 showed no cytotoxic effect on PMN (FIG. 11G). Thus, strong PMN activation by LPS was not a requirement for reactivation of dormant cells and NE signaling promoted PMN-mediated reactivation of dormant cells.

### The Effect of Stress In Vitro and In Vivo on Reactivation of Dormant Tumor Cells

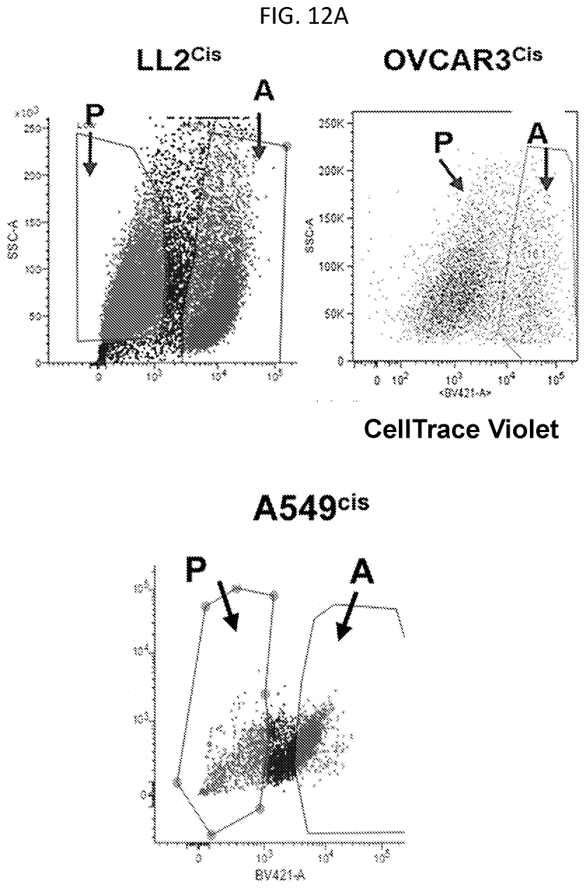

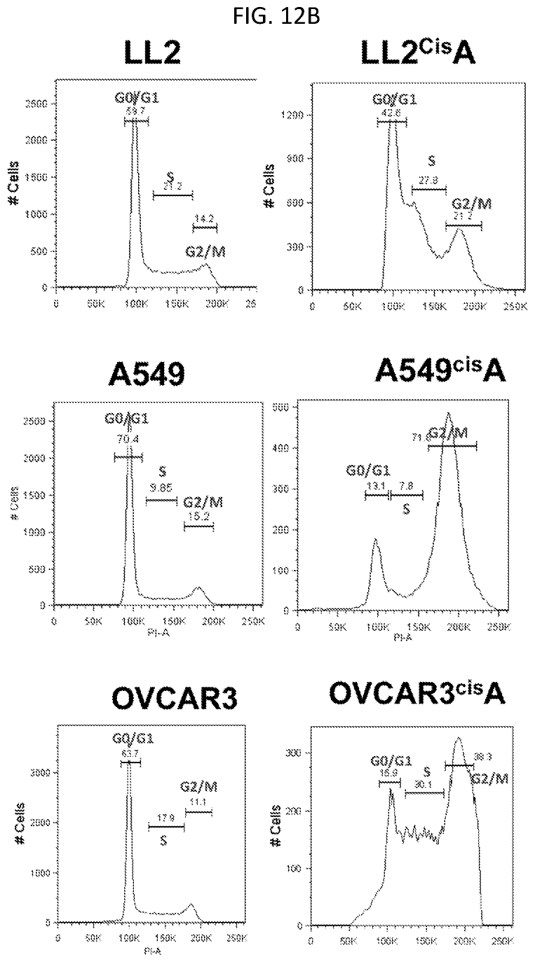

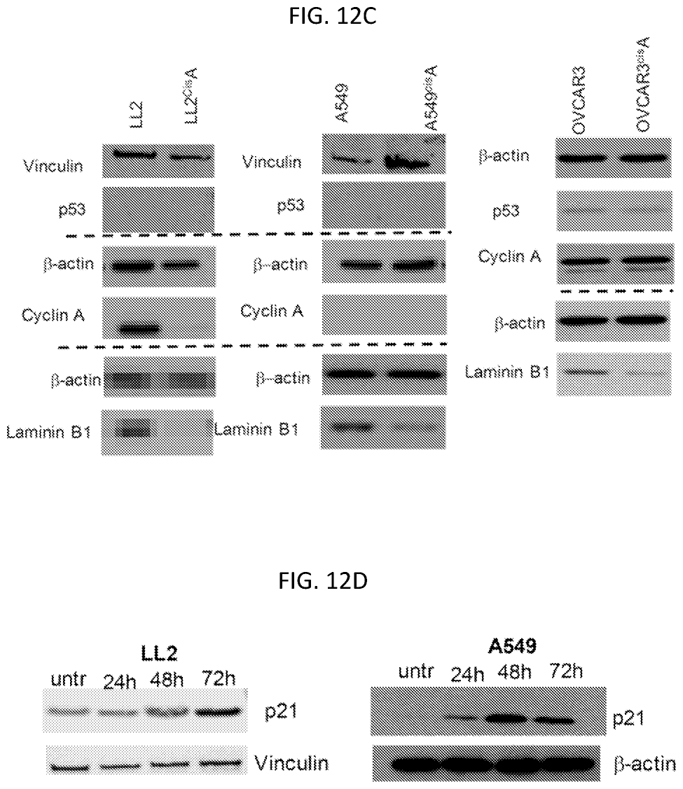

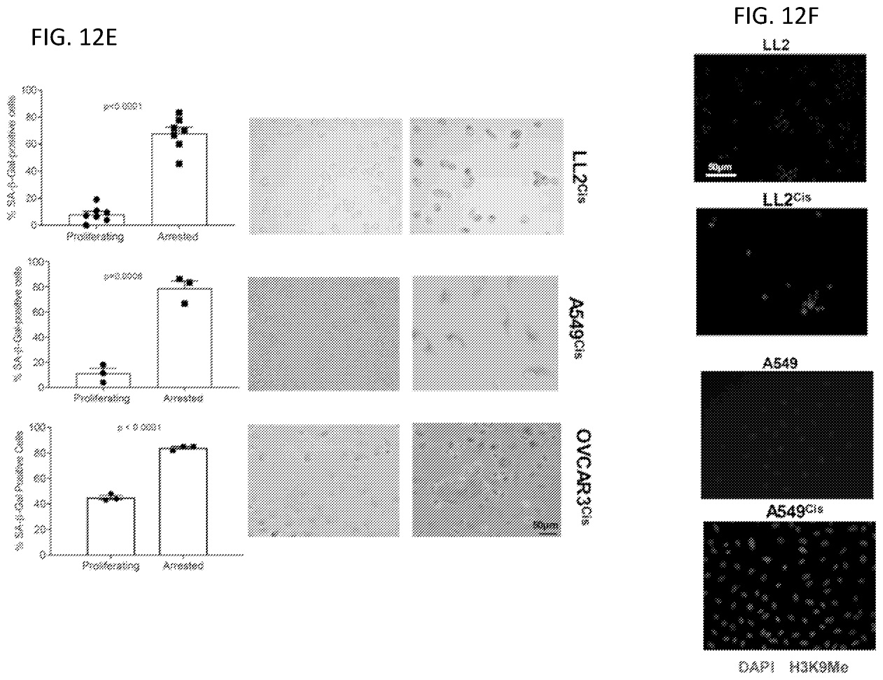

We asked whether PMN could affect tumor cells that underwent chemotherapy-induced senescence. First, we treated mouse lung cancer cells (LL2), human lung cancer cells (A549), and human ovarian cancer cells (OVCAR3) with cisplatin to demonstrate that chemotherapy induced senescence. Cisplatin treatment generated proliferation-arrested cells (LL2^{cis}A; A549^{cis}A; OVCAR3^{cis}A) (FIG. 12A). These cells were arrested in the G2-M phase (FIG. 12B) and had no or low p53 induction, low cyclin A and laminin B1 (FIG. 12C), up-regulated p21 (FIG. 12D), β-galactosidase activity (FIG. 12E), and increased expression of H3K9Me3 (FIG. 12F).

Thus, cisplatin caused a senescence-like phenotype in tumor cells in vitro. This provides a model for evaluating the role of PMN in reactivation of chemotherapy-induced senescence. Mouse PMN in combination with mouse S100A8/A9 activated proliferation of LL2^{cis}A cells (FIG. 2J) and breast carcinoma AT-3 cells that were arrested by the treatment with doxorubicin AT-3^{Dox}A (FIG. 2K). In addition, we found reactivation of A549^{cis}A and OVCAR3^{cis}A cells when cultured with human PMN in combination with human S100A8/A9 (FIG. 2K). NE reactivated dormant mouse and human tumor cells in the presence of PMN, and this effect was abrogated by ICI-118,551 (FIG. 2J and FIG. JK).

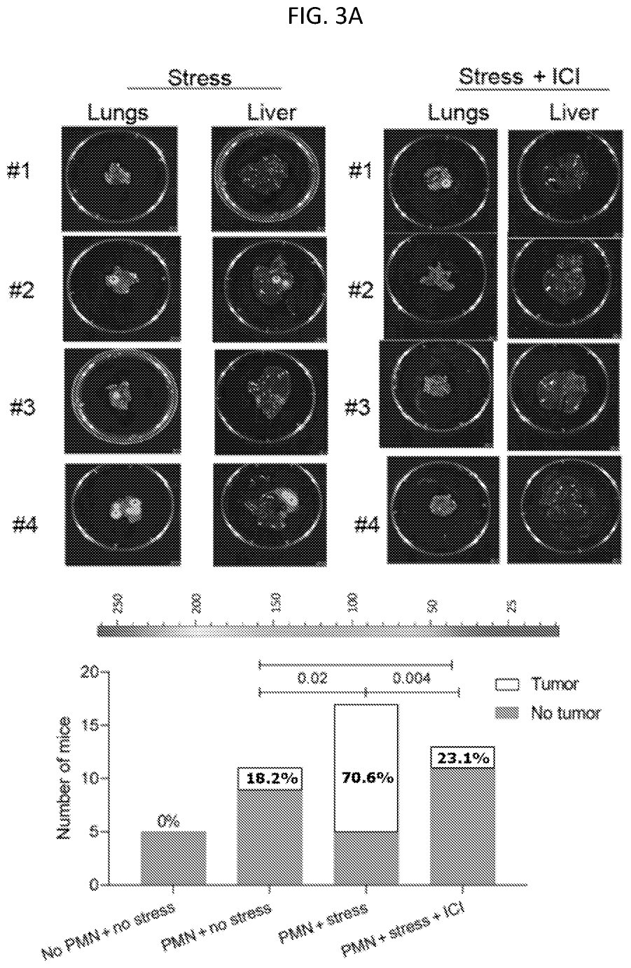

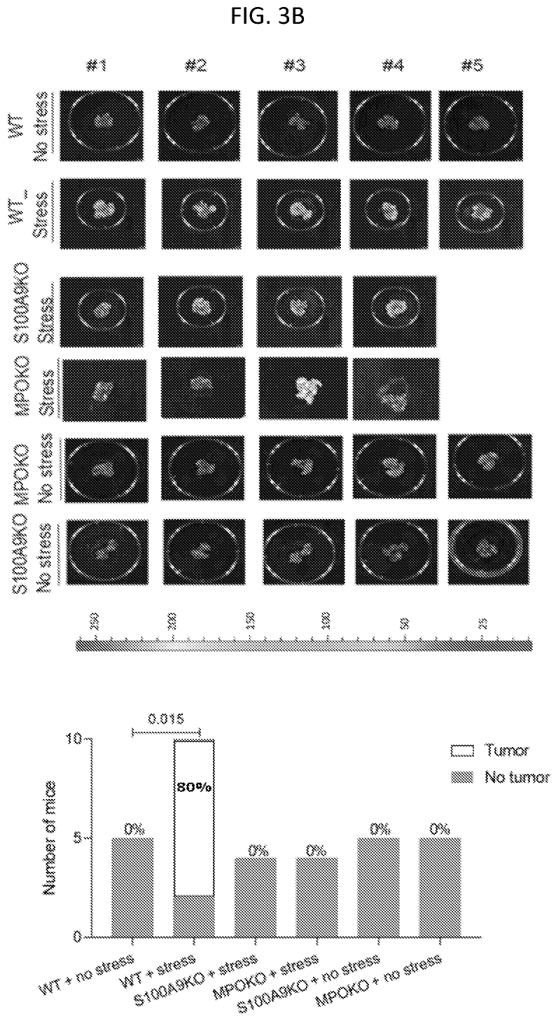

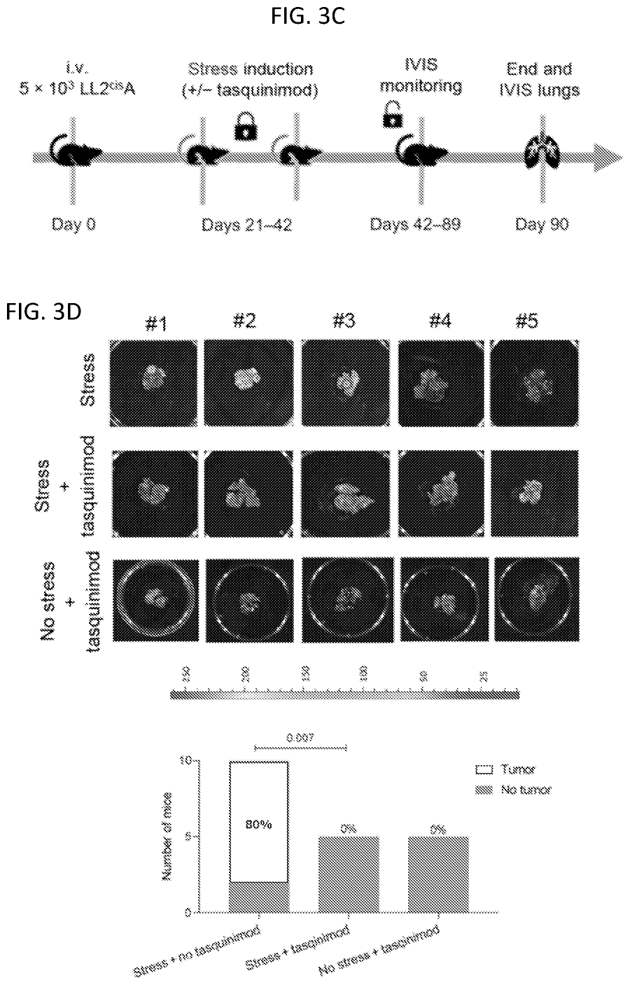

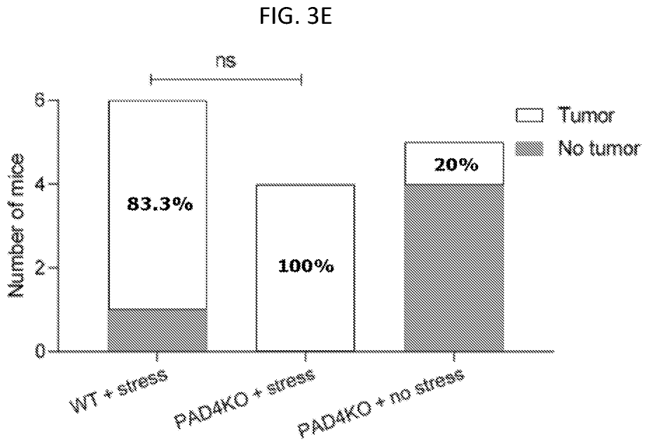

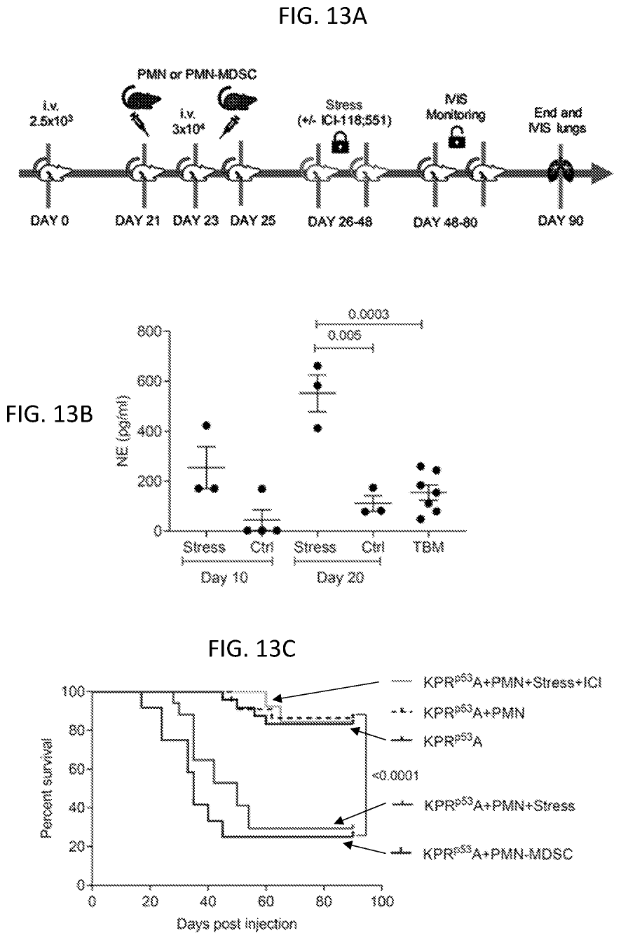

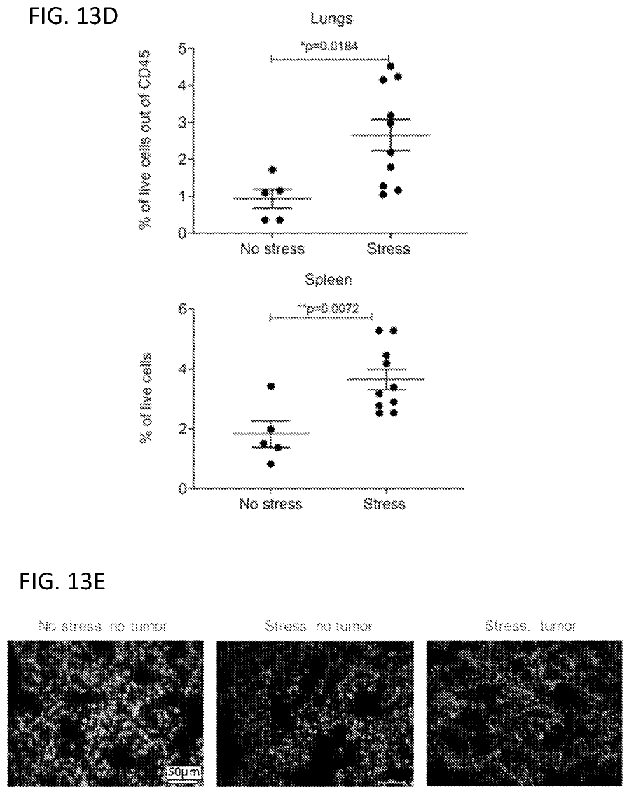

We next evaluated the effect of stress on reactivation of KPr^{p53}A cells in vivo. Stress was induced in vivo by daily immobilization of mice in individual semi-cylindric plastic restrainers (Braintree Scientific) over a 3-week period (32, 33) (FIG. 13A). As expected, this resulted in substantial increase in NE concentration in circulation (FIG. 13B). Intravenous injection of PMN to stressed NOD/SCID tumor-bearing mice caused increased growth of KPr^{p53}A cells in lung and liver in 70.6% of mice, as compared to 18.2% after PMN in injection in mice without stress (P=0.018). Treatment with ICI-118,551 abrogated this effect (FIG. 3A). Using end-point criteria for humane euthanasia of the mice, we also assessed survival of tumor-bearing mice undergoing stress. Administration of PMN to stressed mice bearing KPr^{p53}A cells markedly reduced survival of the mice. The effect was comparable to that observed in nonstressed mice injected with PMN-MDSC from TB mice (FIG. 13C). Last, treatment of mice with ICI-118,551 markedly improved survival (FIG. 13C).

Because LL2 cells can grow in immunocompetent mice, we repeated these experiments in C57BL/6 mice intravenously injected with LL2^{cis}A cells. Stress caused an increase in the number of PMN in both lung and spleen (FIG. 13D and FIG. 13E). In the absence of stress, LL2^{cis}A cells did not form tumor lesions, but in the presence of stress, almost all mice had tumors in lungs. No tumor growth was observed in stressed S100A8/A9 KO mice (FIG. 3B and FIG. 13F).

Besides S100A8/A9, up-regulation of myeloperoxidase (MPO) is also one of the major features of PMN-MDSC (34). To test the possible involvement of MPO in stress-mediated reactivation of dormant tumor cells, we evaluated the effect of stress in MPO KO mice. In the absence of MPO, stress failed to reactivate dormant tumor cells (FIG. 3B). To block S100A8/A9 in these mice, we treated mice with tasquinimod, a drug with selective neutralizing activity against S100A8/9 in vivo (FIG. 3C) (35, 36). Tasquinimod in stressed LL2^{cis}A cell-bearing mice significantly (P=0.007) reduced frequency of tumor lesions (FIG. 3D). Immobilizing stress of PAD4 KO mice, which lack the ability to form NETs, induced tumor growth in all mice injected with LL2^{cis}A cells (FIG. 3E), supporting the conclusion that NETs are unlikely to be involved in stress-induced reactivation of dormant tumor cells.

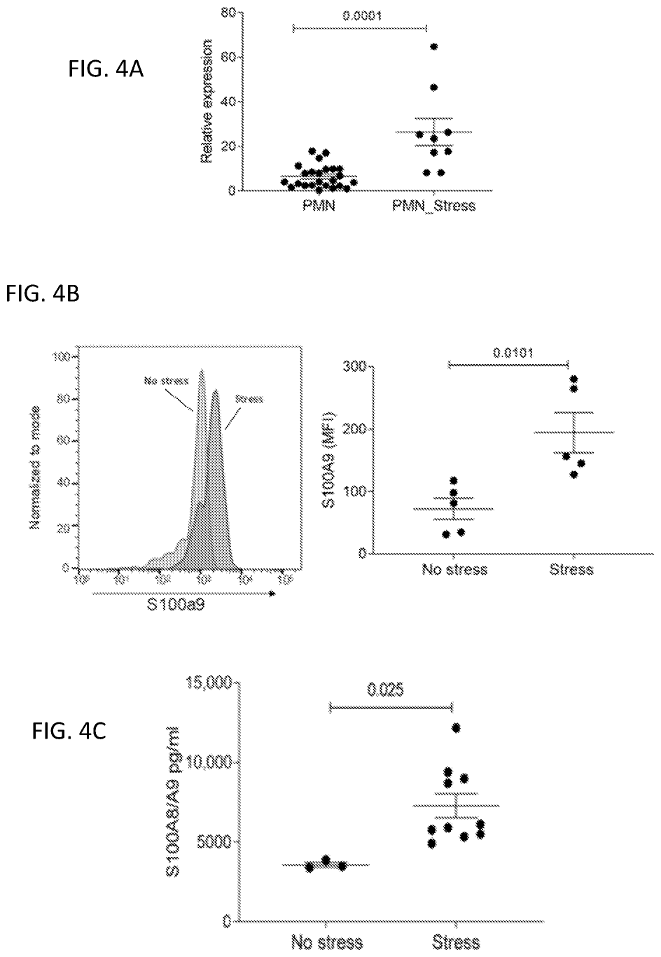

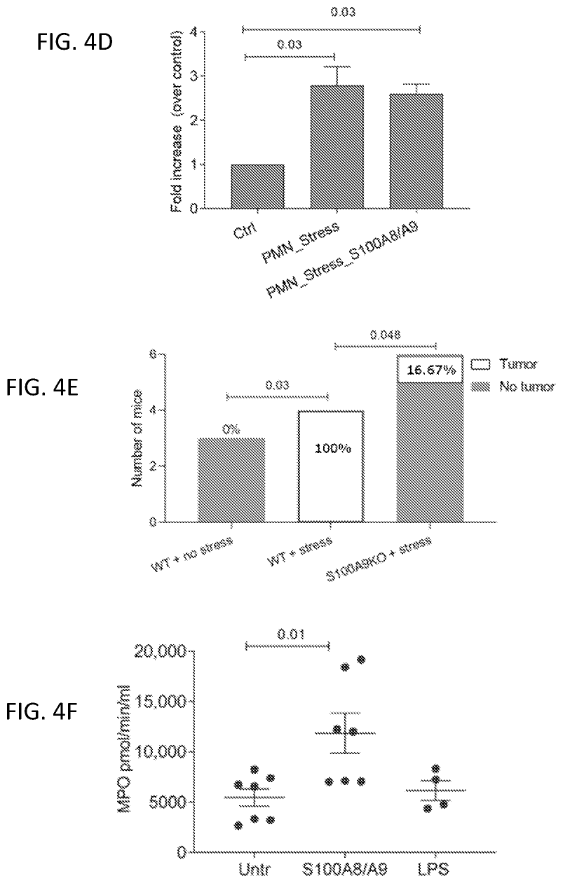

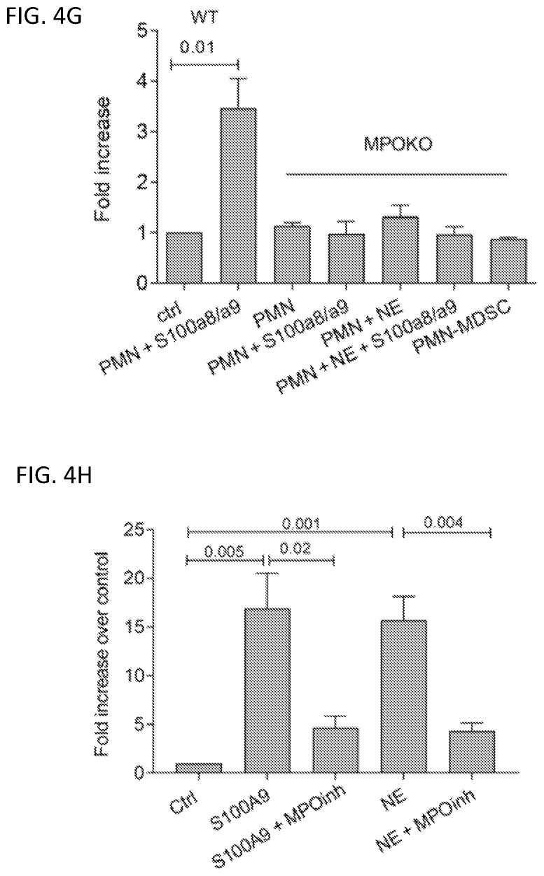

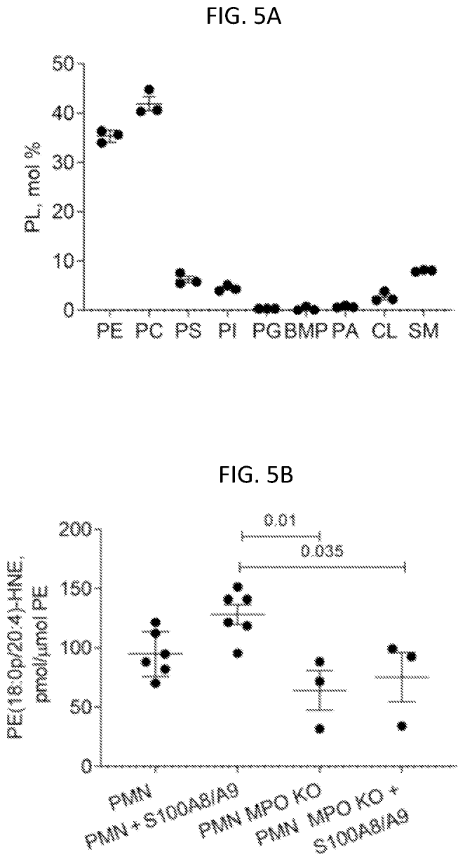

We next measured the expression of S100A9 in PMN in stressed mice. Substantial up-regulation of S100a9 gene (FIG. 4A) and S100A9 protein (FIG. 4B) was observed in PMN from stressed tumor-free mice as compared to control mice. Concentration of S100A8/A9 in sera of mice undergoing stress was substantially higher than that in control mice (FIG. 4C). In addition, PMN isolated from stressed mice activated proliferation of KPr^{p53}A cells without the need for addition of recombinant S100A9 protein (FIG. 4D), suggesting that PMN-expressed S100A9 reaches saturation in the context of stress.

We asked whether this mechanism can regulate reactivation of tumor growth in the spontaneous model of cancer treatment with surgery and chemotherapy. LL2 tumors were established subcutaneously in WT or S100A9 KO C57BL/6 mice. When tumors became palpable, they were resected, and mice were treated with cisplatin (5 mg/kg single dose i.v.) 7 days later. One week after cisplatin treatment, mice were exposed to stress and were imaged by bioluminescence weekly to detect tumor lesions in lungs. Experiments were terminated after 3 weeks, at which point all stressed WT mice had large tumor lesions in lung. In contrast, no tumors were detected in mice not exposed to stress. In addition, only 16.7% of stressed S100A9 KO mice had tumor lesions (FIG. 4E). In this in vivo treatment model, in contrast to the models with transfer of dormant tumor cells, tumor dormancy cannot be formally established because of lack of available cells in tissues for analysis. However, these experiments demonstrate the effect of stress, mediated by S100A9, on tumor progression in the clinically relevant condition of resection.

### S100A8/A9 Regulation of Reactivation of Dormant Tumor Cells is Mediated by Modified Lipids

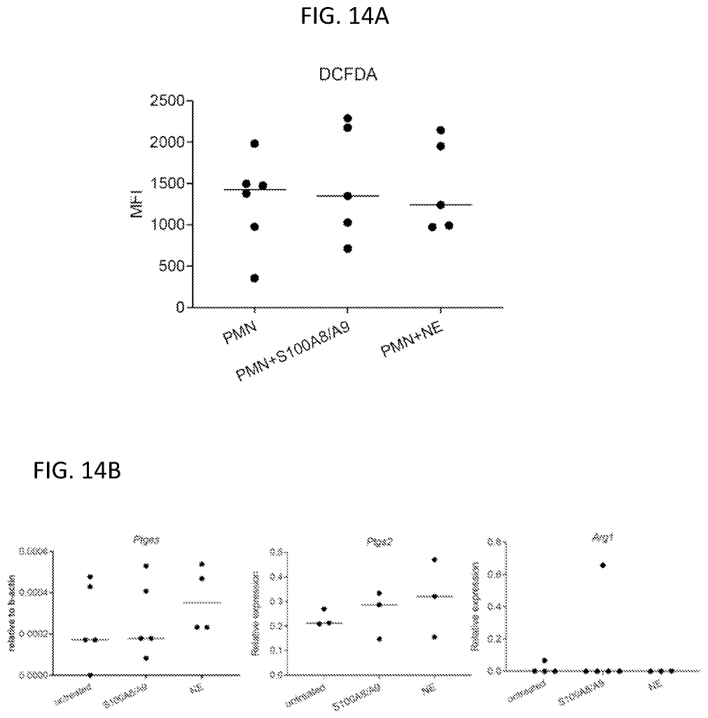

Because S100A8/A9-expressing PMNs were sufficient to mediate dormant tumor cell reactivation, we next sought to understand what changes were induced by S100A8/A9 proteins in PMN that induced the ability to reactivate dormant tumor cells. S100A8/A9 proteins had no effect on ROS production (FIG. 14A), and little or no effect on expression of Arg1, Ptgs2, or Ptges (FIG. 14B). We found that S100A8/A9, but not LPS, activated MPO in PMN (FIG. 4F). PMN derived from MPO KO mice were unable to reactivate dormant cells after exposure to either S100A8/A9 or NE (FIG. 4G). In addition, inhibition of MPO activity with the selective inhibitor 4-aminobenzoic hydrazide 95% (4-ABAH) resulted in abrogation of dormant cell reactivation by PMN (FIG. 4H).

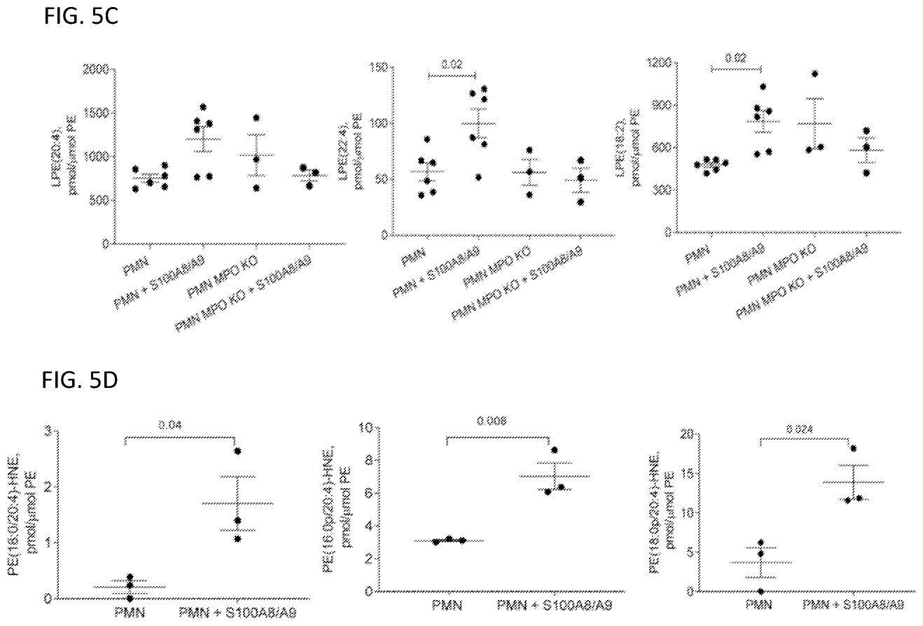

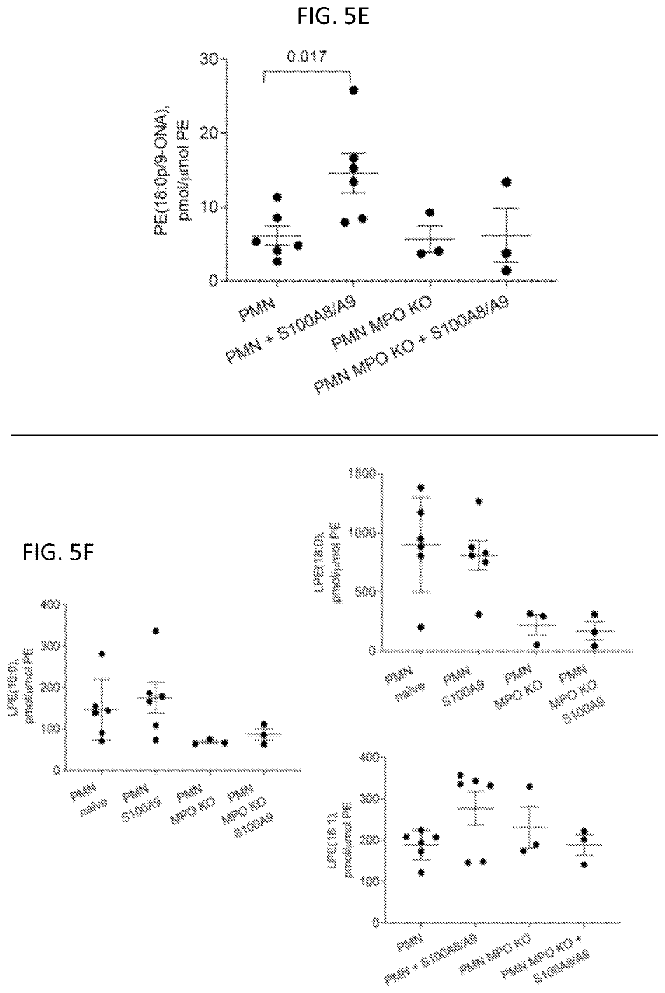

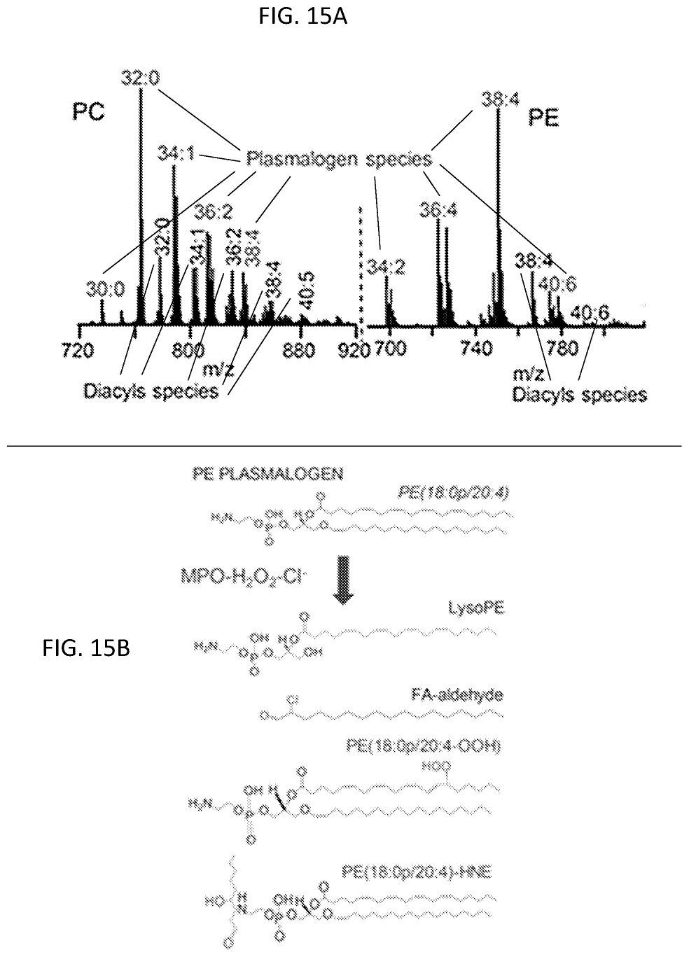

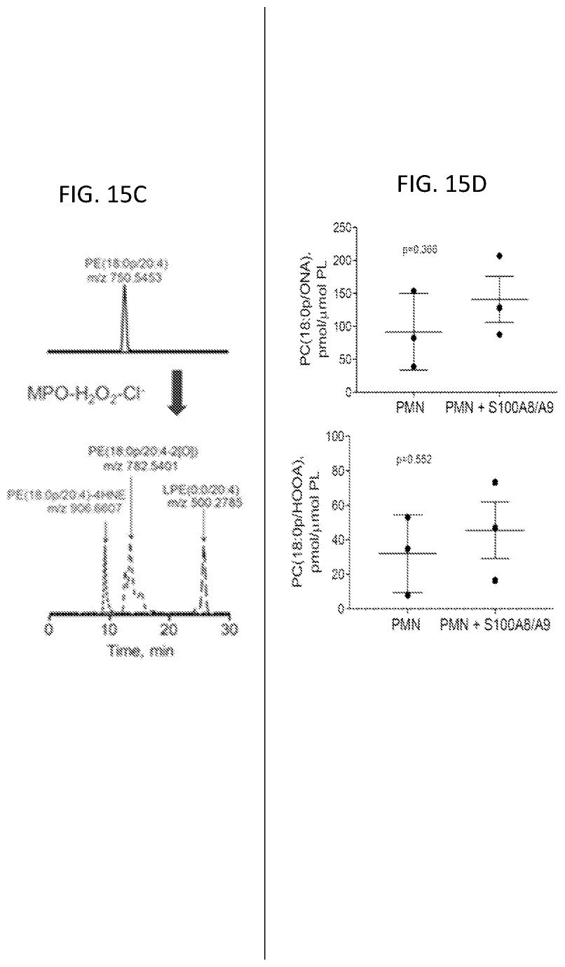

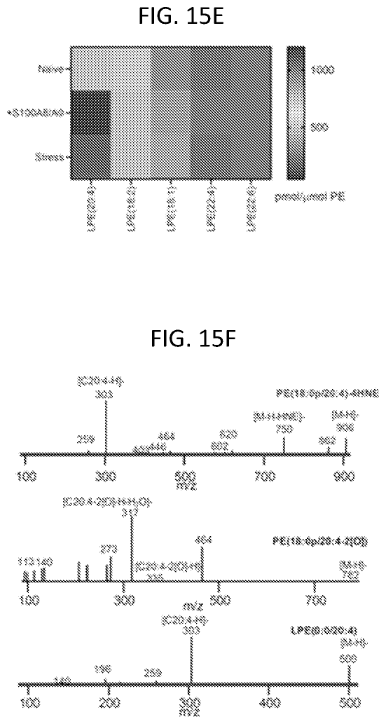

MPO is known to play a major role in lipid modifications, including lipid chlorination/oxidation and hydrolysis (37). Considering that the vinyl ether bond of plasmalogens is a molecular target of the reactive chlorinating species produced by MPO, we analyzed this class of phospholipids. By using liquid chromatography-tandem mass spectrometry (LC-MS/MS), we found that phosphatidylcholine (PC) and phosphatidylethanolamine (PE) were two major classes of phospholipids in S100A8/9-stimulated PMN (FIG. 5A). Plasmalogen alkenyl-acyl species of PE (PE-p) and PC (PC-p) were more predominant than di-acylated PE (PE-d) and PC (PC-d) species (FIG. 15A). PE-p were mostly represented by the molecular species with highly oxidizable arachidonic acid in the sn-2 position. In contrast, PC-p species had saturated and monoenoic acids in the sn-2 position (FIG. 15A). In the presence of chloride and H₂O₂, MPO generates hypochlorous acid (HOCl) that can cause the formation of chlorinated and peroxidized lipids (FIG. 15B and FIG. 15C). HOCl can also attack plasmalogens and hydrolyze a weak alkenyl bond, thus leading to the production of mono-acylated lyso-phospholipid species and aldehydes, particularly 4-hydroxynonenal (4-HNE). 4-HNE can covalently react with amino-groups of proteins and amino-phospholipids, such as PE (38). We detected increased contents of Michael adducts of PE-4HNE in PMN (FIG. 5B) and lyso-PE (LPE) species (FIG. 15C) in mouse (FIG. 5C) and human PMN (FIG. 5D) incubated with S100A8/A9. In contrast, phosphatidylserine (PS) modified by 4-HNE (PS-4-HNE adduct) was not detected. The accumulation of lyso-PE was not observed in MPO-deficient mouse PMN (FIG. 5C). Likewise, the accumulation of PE-4HNE Michael adducts was abrogated in MPO-deficient PMN (FIG. 5E). No significant changes in the content of oxidatively truncated PC species were found after PMN incubation with S100A8/A9 (FIG. S7D). Furthermore, profiles of unsaturated lyso-PE were similar in PMN isolated from stressed mice and PMN treated with S100A8/A9 (FIG. 15E). The content of unsaturated lyso-PE was increased similarly in responses to either stress (in vivo) and exposure to S100A8/A9 (in vitro). In contrast to MPO-dependent accumulation of LPE molecular species containing unsaturated fatty acids (FIG. 5C), treatment with S100A8/A9 did not change the content of LPE containing saturated and monoenic acyl chains in PMN (FIG. 5F). Thus, S100A8/A9 caused marked accumulation of oxidized, oxidatively truncated, and lyso-PE in PMN, and this effect was dependent on MPO.

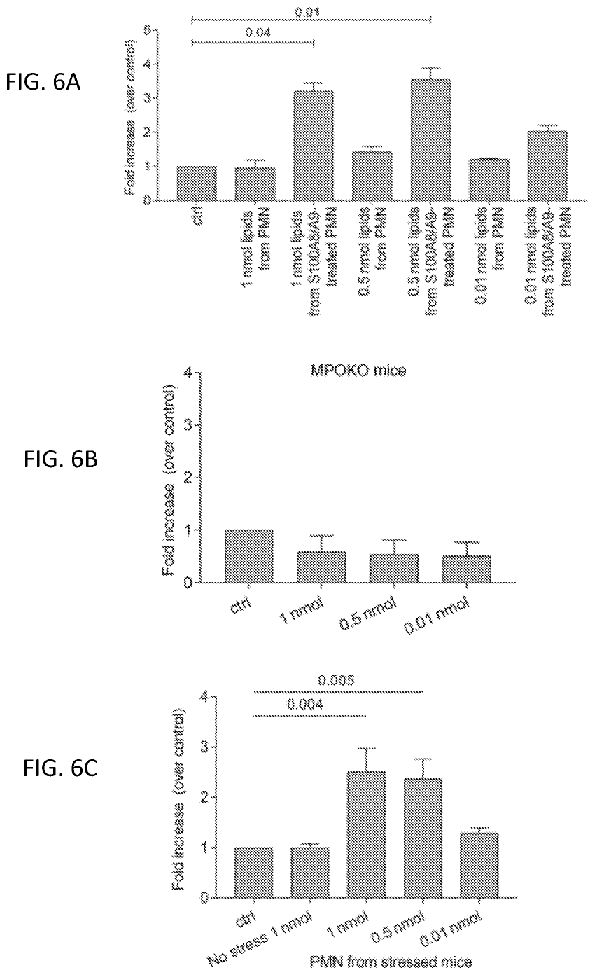

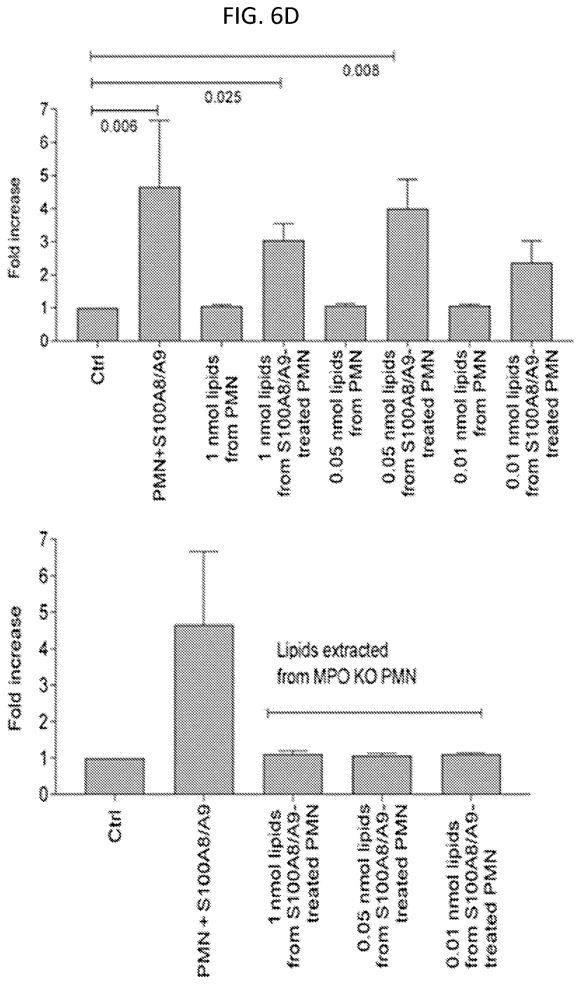

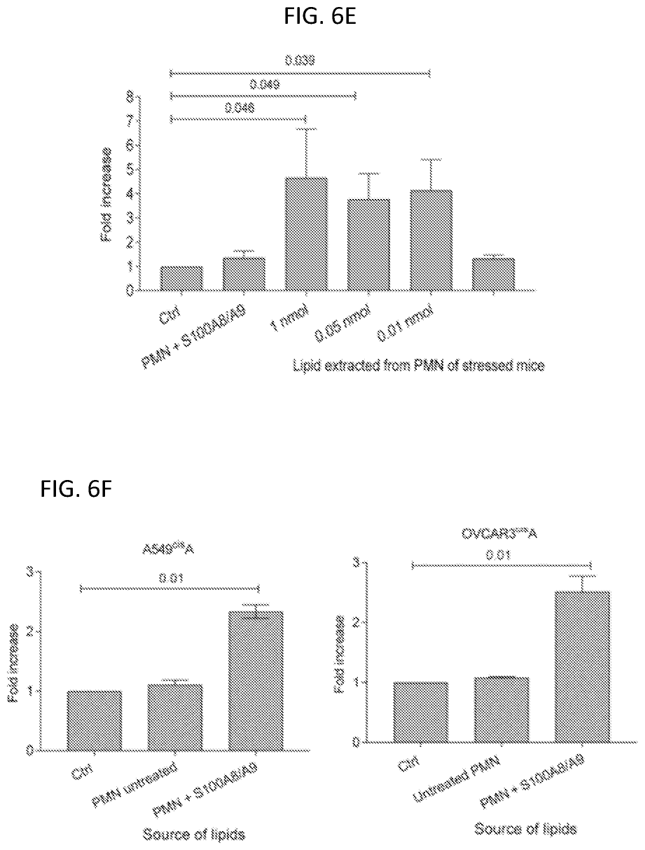

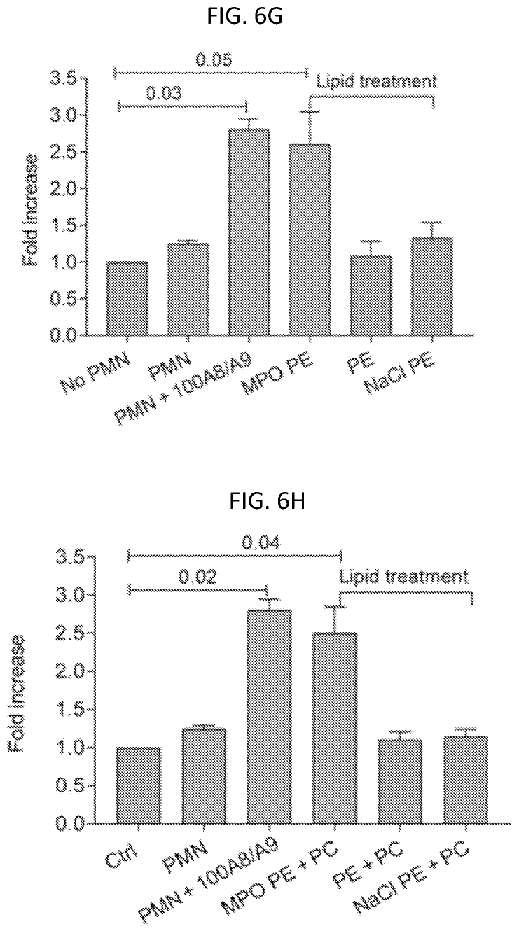

To directly test the role of lipids in reactivation of tumor dormancy, we extracted lipids from PMN and then added them to KPr^{p53}A cells. Lipids extracted from PMN treated with S100A8/A9, but not from control PMN, stimulated proliferation of dormant tumor cells (FIG. 6A). Furthermore, lipids extracted from S100A8/A9-treated MPO-deficient PMN failed to induce proliferation of dormant tumor cells (FIG. 6B). Lipids extracted from PMN isolated from stressed mice reactivated dormant tumor cells (FIG. 6C), and similar results were found in the context of LL2^{cis}A cells by testing different concentrations of lipids extracted from S100A8/A9-treated PMN (FIG. 6D) or PMN from stressed mice (FIG. 6E). Lipids extracted from PMN isolated from human healthy donors and treated with S100A8/A9 activated proliferation of A549^{cis}A or OVCAR3^{cis}A cells. In contrast, lipids isolated from untreated PMN did not reactivate dormant tumor cells (FIG. 6F).

To verify the structure of products formed in the MPO-catalyzed reaction, we incubated PE (18:0p/20:4) with MPO/H₂O₂/NaCl. We found that the major products generated in MPO-driven reaction were represented by PE (18:0p/20:4)-4HNE and lyso-PE containing C20:4 in sn-2 position (GH/20:4). Thus, treatment of pPE-containing lipid sample with MPO/H₂O₂/NaCl recapitulated the nature of PE-4HNE and LPE observed in PMN treated with S100A8/A9. This incubation system, however, also generated hydroperoxy-PE-p species. No lyso-PE-OH/20:4 was formed when PE (18:0p/20:4) was substituted with diacyl PE (18:0/20:4) (FIG. 15F).

Untreated PE or PE treated only with NaCl did not activate proliferation of KPr^{p53}A cells. In contrast, MPO/H₂O2/NaCl-treated PE caused tumor cell expansion comparable to S100A8/A9-treated PMN (FIG. 6G). A similar effect was obtained when mixtures of 18:0p/20:4 and 18:0p/22:6 PE and PC were used (FIG. 6H). A mixture of di-acyl-PE and di-acyl-PC did not have an activating effect on dormant tumor cells. Thus, lipid modification by MPO in stressed or S100A8/9-treated PMNs was sufficient to cause reactivation of dormant tumor cells.

### Transcriptional Signature of Dormant Tumor Cells Reactivated by PMN

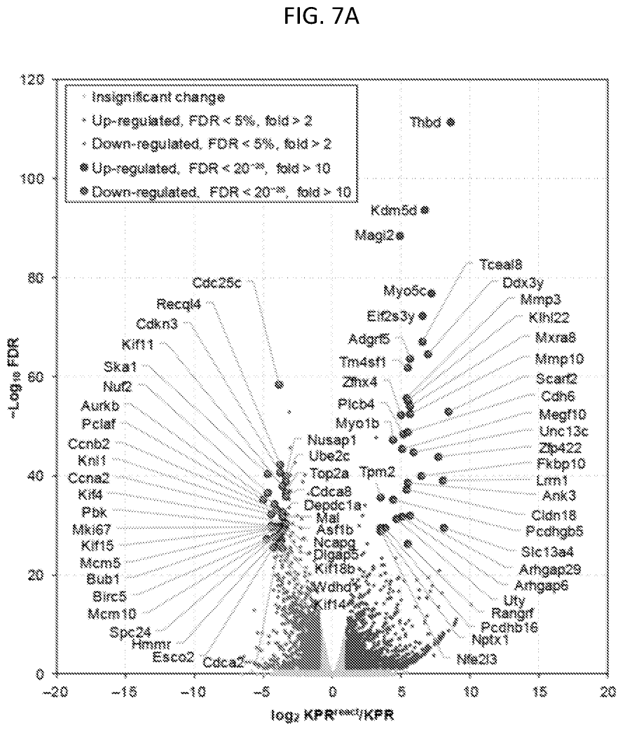

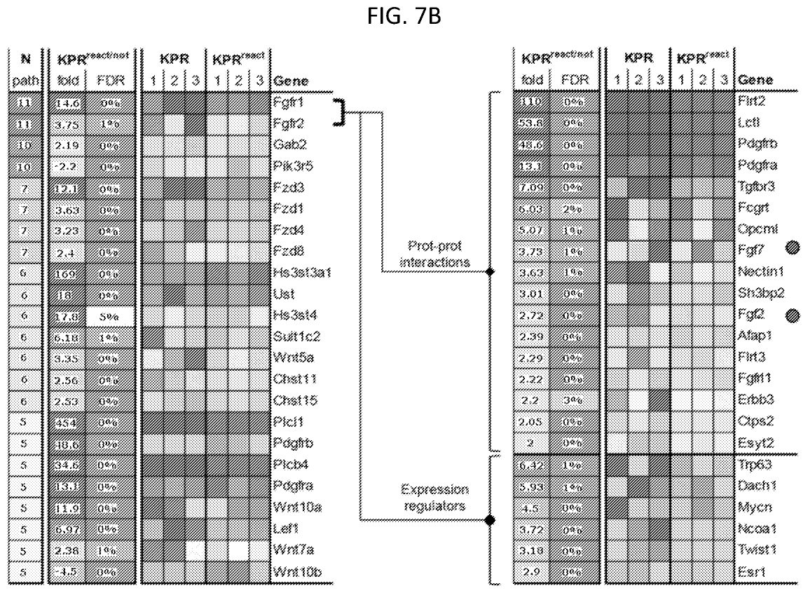

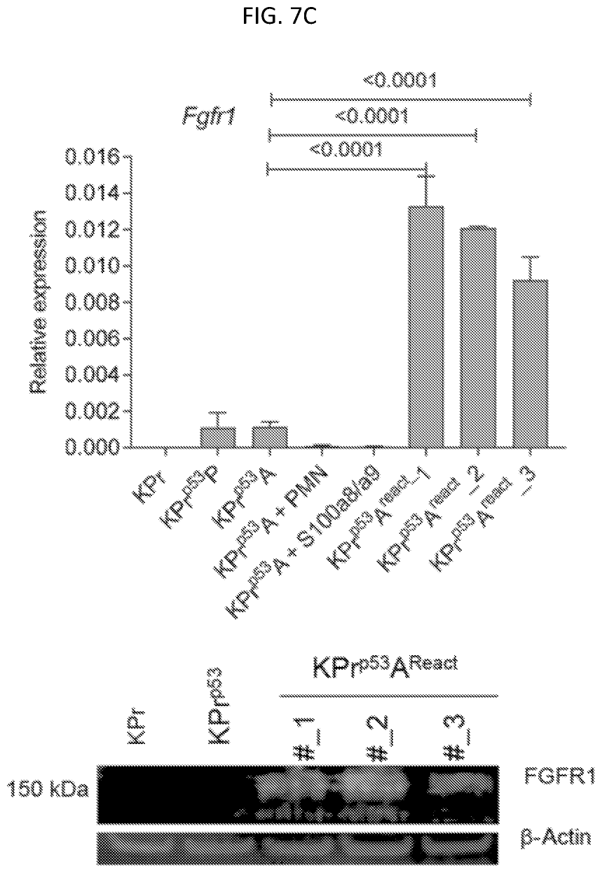

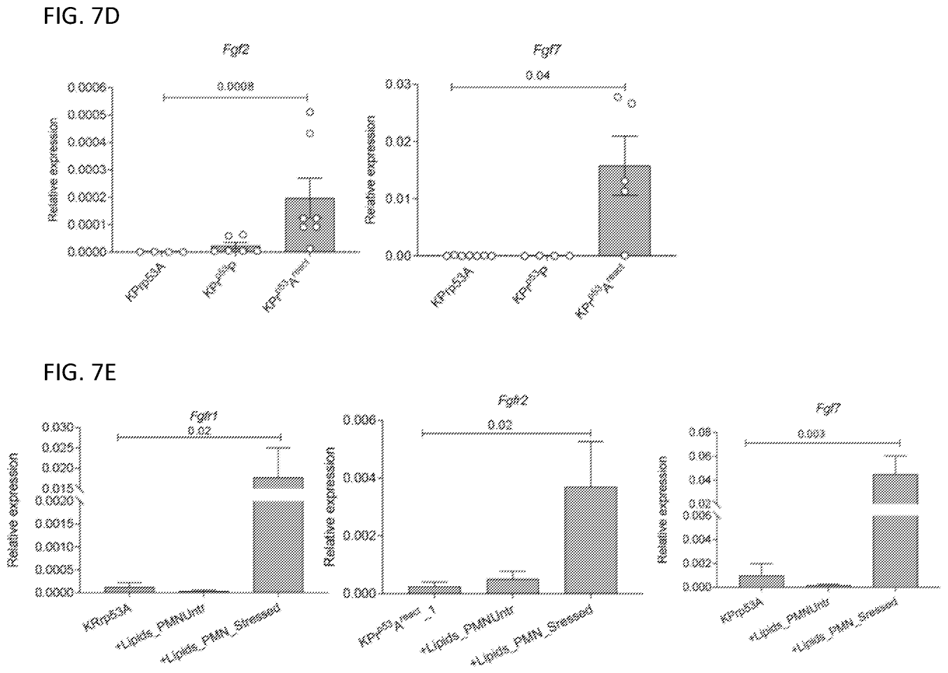

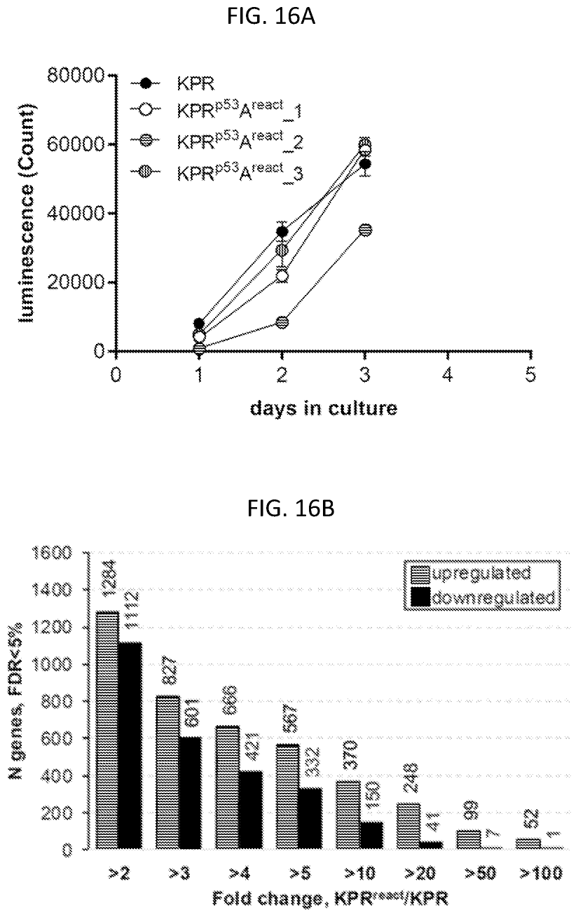

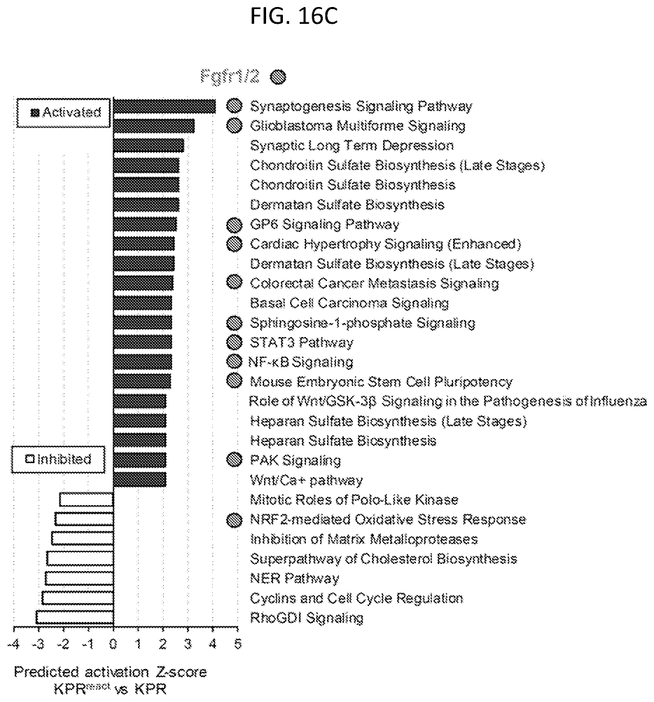

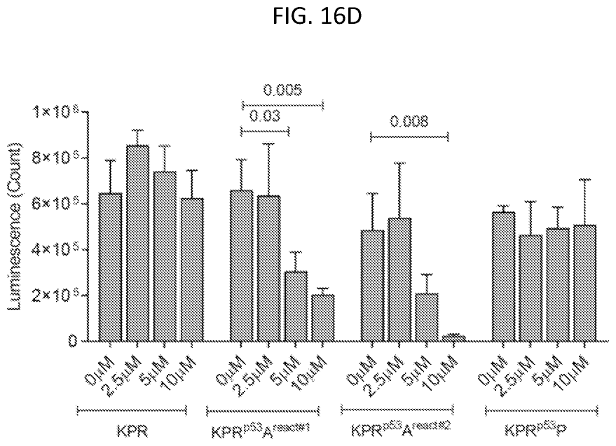

To elucidate the mechanism of tumor cell reactivation, we performed RNA sequencing (RNA-seq) transcriptomic analysis of parent (KPr) and KPr^{p53}A cells that were reactivated by PMN in the presence of S100A8/A9 (KPr^{p53}A^{react}). The proliferation rates of nonarrested versus arrested and reactivated cells were similar (FIG. 16A). We observed a major overall transcriptomic effect, as 2396 genes were changed at least 2-fold and 899 genes at least 5-fold between nonarrested and reactivated tumor cells (false discovery rate, FDR <5%) (FIG. 16B) with the 70 genes changed at least 10-fold (FIG. 7A). Pathway analysis of the genes changed at least two-fold demonstrated considerable change in activity of 27 pathways (Z score >2), with 20 activated in reactivated cells and 7 inhibited (FIG. 16C). Among the 240 genes shared across the 27 pathways, there were 24 genes (FIG. 7B) involved in at least 5 of those pathways, with Fgfr1 and Fgfr2 being involved in the most (11 pathways). Fgfr1 and Fgfr2 were both up-regulated in KPr^{p53}A^{react} as compared with KPr cells (14- and 4-fold, respectively). Interrogating a list of genes involved in protein-protein interactions with FGFR1 and FGFR2 also showed increased expression of fibroblast growth factors 2 and 7 and potential upstream regulators known to increase Fgfr1/2 expression (FIG. 7B). RNA-seq data were validated by quantitative reverse transcription polymerase chain reaction (qRT-PCR) and Western blot analysis (FIG. 7C). In addition, lipid extracts from stressed PMN up-regulated expression of Fgfr1, Fgfr2, and Fgf7 in KPr^{p53}A cells, while lipid extracts from unstressed PMN did not affect arrested cells (FIG. 7D and FIG. 7E).

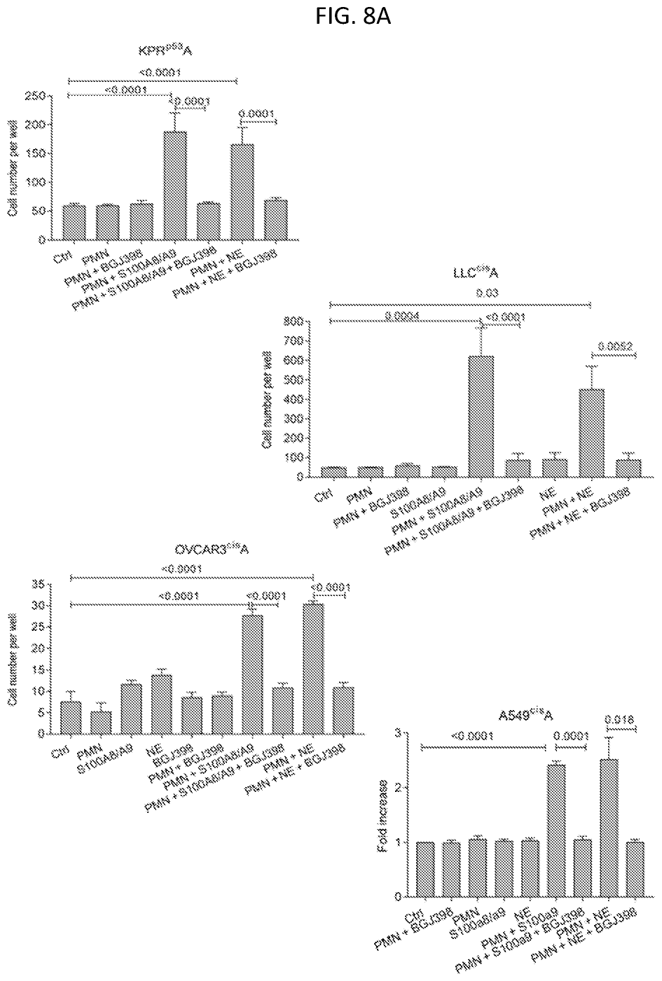

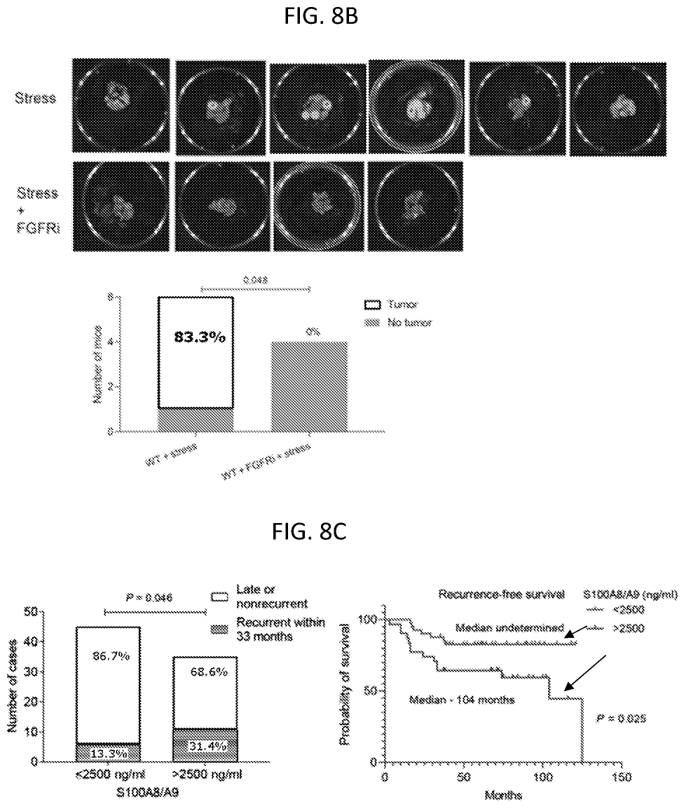

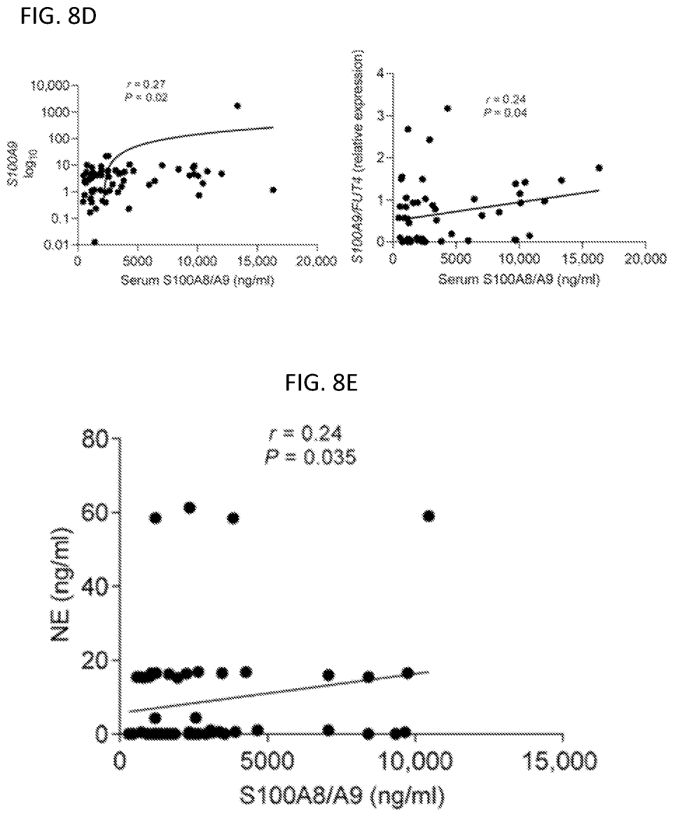

To test a causal role of the FGFR signaling pathway in PMN-mediated reactivation of dormant tumor cells, we used BGJ398—a potent and selective pan-FGFR antagonist (39, 40). Treatment of parental KPr or KPr^{p53}P cells with BGJ398 at 5 or 10 μM did not affect proliferation of tumor cells, whereas treatment of KPr^{p53}A^{react} cells abrogated cell proliferation (FIG. 16D). In addition, blockade of FGFRs in KPr^{p53}A, LLC^{cis}A, OVCAR3^{cis}A, and A549^{cis}A tumor cells with BGJ398 abrogated their reactivation by PMN treated with S100A8/A9 or NE (FIG. 8A). To assess the effect of FGFRi in vivo, LL2^{cis}A cells were intravenously transferred to C57BL/6 mice. Three weeks later, mice were exposed to 3 weeks of stress with or without treatment with BGJ398 (30 mg/kg). Five of six mice that were not treated with FGFRi developed lung tumor lesions at the end of the study. In notable contrast, no mice treated with BGJ398 (0 of 4) had tumor lesions despite exposure to stress (FIG. 8B).

To assess the clinical relevance of the described findings, we evaluated the association between the amount of S100A8/A9 in circulation and the time of recurrence in patients with non-small cell lung cancer (NSCLC). We used archived serum samples from patients with stage I-II NSCLC who underwent complete tumor resection. Samples were collected 3 months after the surgery, before any detectable tumor recurrence. In total, 80 patients were included into this cohort. Seventeen patients had tumor recurrence within 33 months after the surgery (considered as early recurrence) and 63 patients either recurred at a later time point (all more than 37 months after surgery) or did not have recurrence at least 37 months after the surgery. Serum concentrations of S100A8/A9 heterodimers were measured by enzyme-linked immunosorbent assay (ELISA). We used a cutoff of 33 months from time of surgery to separate patients with early recurrence from all other patients. We found that the recurrence rates within 33 months from time of tumor resection were 31.4% (11 of 35) in patients who had serum concentration of S100A8/A9 higher than 2500 ng/ml and 13.3% (6 of 45) in patients who had lower concentrations at their 3-month follow-up time point (P=0.046) (FIG. 8C). No differences were found in the concentration of S100A8/A9 between patients with late recurrence or those who did not recur within the period of observation. We compared recurrence-free survival between patients with high (more than 2500 ng/ml) and low (less than 2500 ng/ml) serum concentrations of S100A8/A9. Patients with high S100A8/A9 concentration had significantly (P=0.025) shorter recurrence-free survival than patients with low concentration of the proteins (FIG. 8C). Frozen buffy coat cells were available from a subset of patients. Therefore, we evaluated a possible link between serum concentrations of S100A8/A9 and expression of S100A9 in buffy coat cells by performing qRT-PCR using RNA extracted directly from pellet of frozen cells, which avoided loss of PMN during thawing. We found correlation (r=0.27, P=0.02) between S100A9 expression by total buffy coat cells and serum concentration of S100A8/A9 (FIG. 8D). To more precisely assess S100A9 expression in PMN, we calculated the ratio of S100A9 and neutrophil-specific FUT4 (encoding CD15) expression in these samples. A positive correlation was observed (r=0.24, P=0.04) between S100A9 and Fut4 expression. Last, serum concentration of NE in a subset of patient samples was measured by ELISA. The serum concentration of NE correlated positively with the serum concentration of S100A8/A9 (r=0.24; P=0.035) (FIG. 8E). Thus, concentration of S100A8/A9 correlated with shorter time to recurrence in patients with NSCLC after curative tumor resection and with the serum concentration of NE in these patients.

| | | Stage at | Patients with recurrence | |
|---|---|---|---|---|
| Age | Gender | diagnosis | after surgery | |
| 50-84 | 30 males; 50 females | Stage I: 68 | <3 | months - 0 |
| | | Stage II: 12 | 3-6 | months - 1 |
| | | | 7-12 | months - 2 |
| | | | 13-18 | months - 8 |
| | | | 19-24 | months - 2 |
| | | | 25-33 | months - 4 |
| | | | >37 | months - 5 |

No recurrence
(>37 months) - 58

The goal of this study was to identify mechanisms that drive reactivation of dormant tumor cells. To this end, we demonstrated that stress-activated PMN were able to reverse tumor cell dormancy caused by genetic up-regulation of p53 or by chemotherapy. Isolation of proliferation-arrested cells allowed us to create experimental conditions of tumor dormancy where cells remained in a nonproliferative state for at least 10 days in vitro or 8 weeks in vivo. Myeloid or lymphoid cells did not activate proliferation of dormant cells even in the presence of proinflammatory cytokines, ER stress, or LPS at concentrations up to 2 μg/ml. Recently, PMN activated by LPS were shown to revert the quiescent state of D2.OR breast cancer cells, which was associated with enhanced processing of Laminin-111 in the basement membrane and release of NET (25). D2.OR cells used in that study were quiescent cells. In the tumor reactivation experiments reported by this previous study, high numbers of tumor cells were injected to the lungs (5×10⁵ cells) and dormancy exit was achieved in the presence of experimental conditions mimicking massive Gram-negative bacterial inflammation (equivalent of about 8 μg/ml LPS). In comparison, LPS concentrations in sera of patients with septic shock are detected within the range of picograms per milliliter (41). These considerations stimulated further search for potential mechanisms involved in pathological reversal of tumor dormancy.

PMN-MDSC, but no other myeloid or lymphoid cells, induced proliferation of dormant cells in vitro and in vivo. One of the most prominent features of PMN-MDSC is the high amount of S100A8 and S100A9 proteins (18). Amounts of S100A8 and S100A9 are much lower in monocytes and are practically undetectable in macrophages, DC, and lymphocytes. Our study demonstrated that the ability of PMN-MDSC to reactivate proliferation of dormant tumor cells was dependent on S100A8/A9. However, PMN-MDSC are absent in adults after complete surgical resection of tumors. To this end, we found that addition of recombinant S100A8/A9 to PMN from tumor-free mice or healthy volunteers enabled these cells to reverse tumor dormancy similar to PMN-MDSC. PMN were required for reversion of tumor dormancy, as addition of S100A8/A9 to dormant tumor cells in the absence of PMN did not affect tumor cell proliferation. These results strongly suggested that the release of S100A8/A9 from PMN is critical for their ability to reverse tumor cell dormancy.

S100A8/A9 proteins lack signal peptides required for the classical Golgi-mediated secretion pathway. Their release is mediated by alternative secretion pathways, which are dependent on src, syk, and tubulin (42). S100A8/A9 have been reported in granules (43), suggesting that they could be released after neutrophil degranulation. However, a subsequent study demonstrated that activation of PMN led to the translocation of S100A8/A9 from the cytosol to the cytoskeleton and membrane before secretion. The secretion was not associated with NETosis or degranulation, and most secreted proteins were found in soluble form or associated with large vesicles (44).

In this study, we found that stress-associated adrenergic hormones caused rapid release of S100A8/A9 from PMN without affecting their viability or NET formation. These results supported the concept that S100A8/A9 release is not associated with PMN degranulation or NETosis. Although increased amount of S100A8/A9 was shared between stress-activated PMN and PMN-MDSC in tumor-bearing mice, it does not appear that these cells are similar. However, in this study, we did not specifically investigate this question.

A role for stress in reactivation of tumor cell dormancy was found by treatment of PMN with NE. Furthermore, the NE- and PMN-induced reactivation of dormant cells was abrogated in S100A8/A9-deficient PMN, indicating that NE-mediated reactivation of dormant cells is mediated via release of S100A8/A9 by PMN. This would be consistent with the epidemiological and clinical studies that provided evidence for links between chronic stress and cancer progression (27, 45).

It appears that autocrine and paracrine effects of S100A8/A9 specifically on PMN are critical for the reactivation of dormant tumor cells. We observed rapid activation of MPO in PMN by S100A8/A9. It was consistent with previous observation that S100A8/A9 could induce HOCl in a cell-free system (46) and that MPO and S100A8/A9 worked synergistically on production of HOCl (47). MPO is important for lipid peroxidation (48). MPO induces peroxidation of phospholipids by causing accumulation of 4-HNE adducts, oxidative truncation, or formation of lyso-PE. Our data indicated that lipid species produced by S100A8/A9-treated or stressed PMN were necessary and sufficient to cause reactivation of dormant tumor cells.

Our data identifies the FGFR pathway as one of the mechanisms by which lipids can support exit of cells from dormancy. FGFR signaling regulates cell cycle progression, migration, metabolism, survival, proliferation, and differentiation of tumor cells (49). Oxidized phospholipids have a pleotropic effect on many cells by affecting signaling mediated by microRNA, cyclic adenosine monophosphate, peroxisome proliferator-activated receptor, or NF-κB (nuclear factor KB) (50). Lyso-phospholipids are described as lipid mediators with a wide variety of functions mediated through G protein (heterotrimetric guanine nucleotide-binding protein)-coupled receptors (51, 52). Oxidatively truncated molecular species PE, including PE-4-HNE Michael adducts, may mediate their effects via receptor binding and activation of cell signaling (53, 54).

Tumor dormancy is a complex system that combines several conditions. Our study was focused on the senescence-like state of tumor cells induced by p53 targeting and by chemotherapy. Our study was not designed to clarify molecular mechanisms of this process. Currently, there are no good models to study tumor dormancy in vivo. A limitation of our study is that we had to use a transfer of dormant tumor cells into mice. Although this approach allows for investigation of the effect of stress and PMN on reactivation of dormant tumor cells, more sophisticated models of tumor cell dormancy in vivo will be needed to clarify the mechanism of this phenomenon.

High concentrations of S100A8/S100A9 are a notable risk factor for a recurrence in patients with NSCLC. These data are in line with two earlier reports. S100A8/S100A9 serum concentrations were found to be reliable surrogate markers for identification of patients at risk for the diagnosis of lung cancer (55). Furthermore, TME-derived S100A8/S100A9 was associated with formation of metastases and had a predictive value for survival rates in melanoma in a similar concentration range found in our patient cohort (56).

Our findings identify several possible therapeutic approaches to reduction of tumor recurrence. First is the targeting of S100A8/A9, which is a central component of the reactivation process. Tasquinimod binds to S100A9 and inhibits its interaction with its receptors TLR4, RAGE, and CD147, reverting the stress-induced reactivation of dormant tumor cells in mice. Tasquinimod has recently emerged as a therapeutic agent for cancer in a limited number of experimental models (57). Earlier data in patients with prostate cancer showed that tasquinimod prolonged progression-free survival compared to placebo (58). However, in a randomized phase III trial, tasquinimod treatment did not affect overall survival, although it increased disease-free survival (59). These data suggest that tasquinimod may be effective in delaying tumor progression but does not affect tumor growth once started. This would be consistent with the potential effect of this drug in our study.

Second, therapy with β-blockers is already used to treat patients with cardiovascular diseases long-term. In our study, inhibition of 02-adrenergic receptors resulted in abrogation of reactivation of dormant tumor cells in mice exposed to stress. This is consistent with clinical observations that patients with lung cancer who used β-blockers showed extended lung cancer survival (60). It has been reported that patients with cancer undergoing β-blocker therapy for associated pathology showed reduced breast cancer recurrence (61) and better survival from ovarian cancer (62). In a meta-analysis over 300,000 patients, β-blocker use was associated with improved survival among patients with ovarian cancer, pancreatic cancer, and melanoma (63). Thus, targeting of stress mediators with β-blockers may provide clinical benefits for patients with cancer by delaying or preventing tumor recurrence.

Third, identification of the exact lipid species responsible for reactivation of dormant cells may result in the development of strategies to neutralize their effect. Identification of the receptors on tumor cells responsible for binding those lipid species could lead to the development of antibodies able to block reactivation of dormant tumor cells.

In conclusion, this study demonstrates that tumor dormancy can be overcome by stress hormone-mediated activation of conventional PMN. This activation is characterized by the release of S100A8/A9 proteins. PMN remained viable and responded to S100A8/A9 proteins in a paracrine and autocrine fashion by activation of MPO and production of oxidized or hydrolyzed phospholipids. These lipids can reactivate dormant tumor cells by up-regulating FGFR signaling. These results provide insight into the mechanisms regulating reactivation of dormant tumor cells and therapeutic strategies to delay or prevent tumor recurrence.

### REFERENCES

1. J. Gong and R. Salgia, Managing patients with relapsed small-cell lung cancer. J. Oncol.

Pract. 14, 359-366 (2018).
2. S. Collaud, T. K. Waddell, K. Yasufuku, A. F. Pierre, G. E. Darling, M. Cypel, Y. R. Rampersaud, et al., Long-term outcome after en bloc resection of non-small-cell lung cancer invading the pulmonary sulcus and spine. J. Thorac. Oncol. 8, 1538-1544 (2013).
3. A. D. Rhim, et al., EMT and dissemination precede pancreatic tumor formation. Cell 148, 349-361 (2012).
4. M. Perego, et al., A slow-cycling subpopulation of melanoma cells with highly invasive properties. Oncogene 37, 302-312 (2018).
5. P. E. Goss and A. F. Chambers, Does tumour dormancy offer a therapeutic target? Nat.

Rev. Cancer 10, 871-877 (2010).
6. J. A. Ewald, et al., Therapy-induced senescence in cancer. J. Natl. Cancer Inst. 102, 1536-1546 (2010).
7. N. Herranz and J. Gil, Mechanisms and functions of cellular senescence. J. Clin. Invest. 128, 1238-1246 (2018).
8. C. Denoyelle, et al., Anti-oncogenic role of the endoplasmic reticulum differentially activated by mutations in the MAPK pathway. Nat. Cell Biol. 8, 1053-1063 (2006).
9. M. Milanovic, et al., Senescence-associated reprogramming promotes cancer stemness.

Nature 553, 96-100 (2018).
10. S. Chakradeo, et al., Is senescence reversible? Curr. Drug Targets 17, 460-466 (2016).
11. L. Lau and G. David, Pro- and anti-tumorigenic functions of the senescence-associated secretory phenotype. Expert Opin. Ther. Targets 23, 1041-1051 (2019).
12. L. J. Gay and I. Malanchi, The sleeping ugly: Tumour microenvironment's act to make or break the spell of dormancy. Biochim. Biophys. Acta 1868, 231-238 (2017).
13. F. Veglia, et al., Myeloid-derived suppressor cells coming of age. Nat. Immunol. 19, 108-119 (2018).
14. D. I. Gabrilovich, Myeloid-derived suppressor cells. Cancer Immunol. Res. 5, 3-8 (2017).
15. P. F. Wang, et al., Prognostic role of pretreatment circulating MDSCs in patients with solid malignancies: A meta-analysis of 40 studies. Onco. Targets. Ther. 7, e1494113 (2018).
16. D. M. Feldser, et al., Stage-specific sensitivity to p53 restoration during lung cancer progression. Nature 468, 572-575 (2010).
17. K. L. Stokes, et al., Natural killer cells limit the clearance of senescent lung adenocarcinoma cells. Oncogenesis 8, 24 (2019).
18. V. Bronte, et al., Recommendations for myeloid-derived suppressor cell nomenclature and characterization standards. Nat. Commun. 7, 12150 (2016).
19. S. Ghavami, et al., S100A8/A9: A Janus-faced molecule in cancer therapy and tumorgenesis. Eur. J. Pharmacol. 625, 73-83 (2009).
20. M. L. Ortiz, et al., Myeloid-derived suppressor cells in the development of lung cancer.

Cancer Immunol. Res. 2, 50-58 (2014).
21. Y. M. He, et al., Transitory presence of myeloid-derived suppressor cells in neonates is critical for control of inflammation. Nat. Med. 24, 224-231 (2018).
22. C. Kerkhoff, et al., The arachidonic acid-binding protein S100A8/A9 promotes NADPH oxidase activation by interaction with p67phox and Rac-2. FASEB J. 19, 467-469 (2005).
23. J. J. Oppenheim and D. Yang, Alarmins: Chemotactic activators of immune responses.

Curr. Opin. Immunol. 17, 359-365 (2005).
24. M. P. Manitz, et al., Loss of S100A9 (MRP14) results in reduced interleukin-8-induced CD11b surface expression, a polarized microfilament system, and diminished responsiveness to chemoattractants in vitro. Mol. Cell. Biol. 23, 1034-1043 (2003).
25. J. Albrengues, et al., Neutrophil extracellular traps produced during inflammation awaken dormant cancer cells in mice. Science 361, eaao4227 (2018).
26. O. E. Sorensen and N. Borregaard, Neutrophil extracellular traps—the dark side of neutrophils. J. Clin. Invest. 126, 1612-1620 (2016).
27. V. F. Chiriac, et al., Psychological stress and breast cancer incidence: A systematic review. Clujul Med. 91, 18-26 (2018).
28. M. M. S. Obradovic, et al., Glucocorticoids promote breast cancer metastasis. Nature 567, 540-544 (2019).
29. A. H. Zahalka, et al., Adrenergic nerves activate an angio-metabolic switch in prostate cancer. Science 358, 321-326 (2017).
30. A. S. Nagaraja, et al., Sustained adrenergic signaling leads to increased metastasis in ovarian cancer via increased PGE2 synthesis. Oncogene 35, 2390-2397 (2016).
31. D. Beis, et al., The role of norepinephrine and β-adrenergic receptors in acute stress-induced changes in granulocytes and monocytes. Psychosom. Med. 80, 649-658 (2018).
32. A. C. Campos, et al., Animal models of anxiety disorders and stress. Braz. J. Psychiatry 35 (Suppl. 2), S101-5111 (2013).
33. A. Banagozar Mohammadi, et al., Sericin alleviates restraint stress induced depressive- and anxiety-like behaviors via modulation of oxidative stress, neuroinflammation and apoptosis in the prefrontal cortex and hippocampus. Brain Res. 1715, 47-56 (2019).
34. J. I. Youn, et al., Characterization of the nature of granulocytic myeloid-derived suppressor cells in tumor-bearing mice. J. Leukoc. Biol. 91, 167-181 (2012).
35. L. Shen, et al., Tasquinimod modulates suppressive myeloid cells and enhances cancer immunotherapies in murine models. Cancer Immunol. Res. 3, 136-148 (2015).
36. E. Raymond, et al., Mechanisms of action of tasquinimod on the tumour microenvironment. Cancer Chemother. Pharmacol. 73, 1-8 (2014).
37. E. N. D. Palladino, et al., The chlorinated lipidome originating from myeloperoxidase-derived HOCl targeting plasmalogens: Metabolism, clearance, and biological properties.

Arch. Biochem. Biophys. 641, 31-38 (2018).
38. M. Guichardant, et al., Covalent modifications of aminophospholipids by 4-hydroxynonenal. Free Radic. Biol. Med. 25, 1049-1056 (1998).
39. S. K. Pal, et al., Efficacy of BGJ398, a fibroblast growth factor receptor 1-3 inhibitor, in patients with previously treated advanced urothelial carcinoma with FGFR3 alterations. Cancer Discov. 8, 812-821 (2018).
40. M. Javle, et al., Phase II study of BGJ398 in patients with FGFR-altered advanced cholangiocarcinoma. J. Clin. Oncol. 36, 276-282 (2018).
41. R. L. Danner, et al., Endotoxemia in human septic shock. Chest 99, 169-175 (1991).
42. C. Ryckman, et al., Role of S100A8 and S100A9 in neutrophil recruitment in response to monosodium urate monohydrate crystals in the air-pouch model of acute gouty arthritis. Arthritis Rheum. 48, 2310-2320 (2003).
43. D. F. Stroncek, et al., The subcellular distribution of myeloid-related protein 8 (MRP8) and MRP14 in human neutrophils. J. Transl. Med. 3, 36 (2005).
44. M. R. Tardif, et al., Secretion of S100A8, S100A9, and S100A12 by neutrophils involves reactive oxygen species and potassium efflux. J. Immunol. Res. 2015, 296149 (2015).
45. M. Moreno-Smith, et al., Impact of stress on cancer metastasis. Future Oncol. 6, 1863-1881 (2010).
46. A. Bøyum, et al., Calprotectin (S100A8/S100A9) and myeloperoxidase: Co-regulators of formation of reactive oxygen species. Toxins 2, 95-115 (2010).
47. J. C. Simard, et al., Induction of neutrophil degranulation by S100A9 via a MAPK-dependent mechanism. J. Leukoc. Biol. 87, 905-914 (2010).
48. A. Strzepa, et al., Myeloperoxidase: A new player in autoimmunity. Cell. Immunol. 317, 1-8 (2017).
49. A. Desai, A. A. Adjei, FGFR signaling as a target for lung cancer therapy. J. Thorac. Oncol. 11, 9-20 (2016).
50. V. Bochkov, et al., Pleiotropic effects of oxidized phospholipids. Free Radic. Biol. Med. 111, 6-24 (2017).
51. J. Rolin and A. A. Maghazachi, Effects of lysophospholipids on tumor microenvironment. Cancer Microenviron. 4, 393-403 (2011).
52. K. Makide, et al., Novel lysophosphoplipid receptors: Their structure and function. J. Lipid Res. 55, 1986-1995 (2014).
53. C. M. Spickett, The lipid peroxidation product 4-hydroxy-2-nonenal: Advances in chemistry and analysis. Redox Biol. 1, 145-152 (2013).
54. R. J. Schaur, et al., 4-hydroxy-nonenal—A bioactive lipid peroxidation product. Biomolecules 5, 2247-2337 (2015).
55. S. Blanco-Prieto, et al., Serum calprotectin, CD26 and EGF to establish a panel for the diagnosis of lung cancer. PLOS ONE 10, e0127318 (2015).
56. N. B. Wagner, et al., Tumor microenvironment-derived S100A8/A9 is a novel prognostic biomarker for advanced melanoma patients and during immunotherapy with anti-PD-1 antibodies. J. Immunother. Cancer 7, 343 (2019).
57. E. Kallberg, et al., S100A9 interaction with TLR4 promotes tumor growth. PLOS ONE 7, e34207 (2012).
58. R. Pili, et al., Phase II randomized, double-blind, placebo-controlled study of tasquinimod in men with minimally symptomatic metastatic castrate-resistant prostate cancer. J. Clin. Oncol. 29, 4022-4028 (2011).
59. C. Sternberg, et al., Randomized, double-blind, placebo-controlled phase III study of tasquinimod in men with metastatic castration-resistant prostate cancer. J. Clin. Oncol. 34, 2636-2643 (2016).
60. A. Zingone, et al., Relationship between anti-depressant use and lung cancer survival. Cancer Treat. Res. Commun. 10, 33-39 (2017).
61. W. K. Childers, et al., O-blockers reduce breast cancer recurrence and breast cancer death: A meta-analysis. Clin. Breast Cancer 15, 426-431(2015).
62. M.-H. Baek, et al., Impact of beta blockers on survival outcomes in ovarian cancer: A nationwide population-based cohort study. J. Gynecol. Oncol. 29, e82 (2018).
63. Z. Na, et al., The effects of beta-blocker use on cancer prognosis: A meta-analysis based on 319,006 patients. Onco. Targets. Ther. 11, 4913-4944 (2018).
64. M. Frosch, et al., Myeloid-related proteins 8 and 14 are specifically secreted during interaction of phagocytes and activated endothelium and are useful markers for monitoring disease activity in pauciarticular-onset juvenile rheumatoid arthritis. Arthritis Rheum. 43, 628-637 (2000).
65. B. Langmead and S. L. Salzberg, Fast gapped-read alignment with Bowtie 2. Nat. Methods 9, 357-359 (2012).
66. B. Li and C. N. Dewey, RSEM: Accurate transcript quantification from RNA-seq data with or without a reference genome. BMC Bioinformatics 12, 323 (2011).
67. M. I. Love, et al., Moderated estimation of fold change and dispersion for RNA-seq data with DESeq2. Genome Biol. 15, 550 (2014).

All publications cited in this specification, as well as U.S. Provisional Patent Application No. 63/006,312, filed Apr. 7, 2020, are incorporated herein by reference. While the invention has been described with reference to particular embodiments, it will be appreciated that modifications can be made without departing from the spirit of the invention. Such modifications are intended to fall within the scope of the appended claims.

1. A method of inhibiting reactivation of dormant tumor cells in a subject, the method comprising
a) inhibiting or reducing S100A8/A9 in the subject;
b) inhibiting or reducing FGFR in the subject; and/or
c) inhibiting or reducing myeloperoxidase (MPO) in the subject.

2. The method according to claim 1, comprising administering an S100A8/A9 inhibitor.

3. The method according to claim 2, wherein the inhibitor is tasquinimod, paquinimod, bromodomain inhibitor-JQ1, or arachidonic acid.

4. The method according to claim 1, comprising reducing the level of S100A8 or S100A9 mRNA in the subject.

5. The method according to claim 4, wherein the S100A8 or S100A9 mRNA levels are reduced using siRNA.

6. The method according to claim 1, comprising administering an anti-A100A8/9 antibody.

7. (canceled)

8. The method according to claim 1, comprising administering an FGFR inhibitor.

9. The method according to claim 8, wherein the inhibitor is Infigratinib phosphate (BGJ398), Erdafitinib, Deranzantinib hydrochloride, Rogaratinib, HMPL-453, Futibatinib, PRN-1371, LY-2874455, BPI-17213, CPL-043, or ASP-5878.

10. The method according to claim 1, wherein the FGFR is FGFR1, FGFR2, and/or FGF7.

11. The method according to claim 1, comprising reducing the level of FGFR mRNA in the subject.

12. The method according to claim 11, wherein the FGFR mRNA levels are reduced using siRNA.

13. (canceled)

14. The method according to claim 1, comprising administering an MPO inhibitor.

15. The method according to claim 1, comprising reducing the level of MPO mRNA in the subject.

16. The method according to claim 15, wherein the MPO mRNA levels are reduced using siRNA.

17. A method comprising:
comparing the level of S100A8 or S100A9 in a sample obtained from a subject, wherein an increase in the level of S100A8 or S100A9 as compared to a control indicates a greater risk of reactivation of, or presence of reactivated, dormant tumor cells in the subject;
treating the subject with an inhibitor of S100A8 or S100A9, FGFR, and/or MPO.

18. The method according to claim 17, wherein
a) a level of 2500 ng/mL or higher is indicative of an increased risk of reactivation of dormant tumor cells in the subject;
b) the sample is serum, plasma, or whole blood;
c) wherein the cancer is lung or ovarian cancer.

19. (canceled)

20. The method according to claim 17, further comprising treating the subject with a chemotherapeutic agent.

21-29. (canceled)

30. A method of inhibiting the recurrence of cancer in a subject, comprising inhibiting stress-induced β-adrenergic pathway signaling.

31. The method according to claim 30, the method comprising inhibiting or reducing S100A8/A9 in the subject.

32. The method according to claim 30, comprising administering an S100A8/A9 inhibitor.

33. (canceled)
